SEQUENCE LISTING (11 sequences)

SEQ ID NO: 1 - DNA, 6418 bp, synthetic construct
  misc_feature 1..6418: note = EXPRESSION CASSETTE
  source 1..6418: mol_type = other DNA
gcggccgccctgcaggccatcaaaaatataagtgcggtatgagttctcgctataacgatcggagatacaggggcgtacccagtaagcaactccatagcatacgtgttctcatccattagagagttagtatatacaatacaatatttctgaccaataagaggttttagaatattacgattagtgatgagaggaataatttcacccggattaatcttggcaaatgttctgttaggaatagcagctagaacattctttgtagtcttgtaatcaacgatggcggcatcctcgaacttattattttttgagatatcctcgataattttatgccattcagccattgttatttattatatatgttttttaataagacatctattggaataaacttgacattagcattttattcttattacaaaatataaaatataaaatataaaataaaatatacaatccaatactcacataatccaactcactcgaacactatttttccaattacgataacaatattgcagacctgcaggtagtaacccttaattttattaaaataaccgcaatttatttggcggcaacacaggatctctcttttaagttactctctattacatacgttttccatctaaaaattagtagtattgaacttaacggggcatcgtattgtagttttccatattggcgcgcccaaaaaattgaaattttattttttttttttggaatataaataatgccgccaccatgacggccctgacagaaggtgcgaagctgttcgagaaggagattccctatatcacagaattggagggggatgtagagggtatgaagtttatcatcaaaggcgaagggacaggggatgcaacaactggaacaattaaggctaagtacatttgcacgaccggcgacgtcccggtgccctggtccacgctcgtcaccacgctcacgtacggagcccagtgctttgccaaatatggccctgaacttaaagacttctacaagtcgtgtatgccggagggatacgtgcaagagaggacgatcacctttgaaggtgacggagtattcaaaacaagagcggaggtgacgttcgagaatggatcggtctataaccgggtcaagctcaacggacagggctttaagaaagatggacacgtccttgggaagaatttggagttcaatttcaccccgcattgtctttacatctggggtgatcaggcgaatcacgggttgaaatcagcgttcaagatcatgcacgagattacggggagcaaagaggactttatcgtggcagaccacactcagatgaacactccaatcggagggggtcccgtacacgtacccgagtatcatcacctgaccgtctggacatcgtttggaaaagaccctgacgacgatgaaactgatcatctcaacattgtggaagtgatcaaggcggtggacttggaaacataccgcgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatttttatggcgcgcctagtaacccttaattttattaaaataaccgcaatttatttggcggcaacacaggatctctcttttaagttactctctattacatacgttttccatctaaaaattagtagtattgaacttaacggggcatcgtattgtagttttccatattttaaaaaattgaaattttattttttttttttggaatataaatatccctatcagtgatagagagccgccaccatgtttgttttccttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttctgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttgaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatttttattaaatttaaatgcgatcgcttgttatttttataaaaacaagaacggtacggcgatatttatttttttctaaaacatctaaccgaagtagtggtatgataaaaatgtagtgtaattgttatatagtgtaacacgaatgcagtttgggagtattcgttgtgctacaaatatatttccattcttcgtggcatttttagctataaaattatatggggttgtctggtttaatatttcggattctcctacccaatagcctcgtctaaggcttcttaaaaaacttaactcgtttggagtctctatcttaattagatccgaatttggatttagagctttgcatgtattacgtccttcctcccaggtttttcgatcagtagataaatggatacatttattattatagcttatccagtcagtaggacaatcttctctaatatatgcatcttttatcgttttatctattgatggtgtatgaactaatttacgttctacaacttttaatagtgtcgctgtaaaaataattcgcgatcgcgcggccgc

SEQ ID NO: 2 - DNA, 6482 bp, synthetic construct
  misc_feature 1..6482: note = EXPRESSION CASSETTE
  source 1..6482: mol_type = other DNA
gcggccgccctgcaggccatcaaaaatataagtgcggtatgagttctcgctataacgatcggagatacaggggcgtacccagtaagcaactccatagcatacgtgttctcatccattagagagttagtatatacaatacaatatttctgaccaataagaggttttagaatattacgattagtgatgagaggaataatttcacccggattaatcttggcaaatgttctgttaggaatagcagctagaacattctttgtagtcttgtaatcaacgatggcggcatcctcgaacttattattttttgagatatcctcgataattttatgccattcagccattgttatttattatatatgttttttaataagacatctattggaataaacttgacattagcattttattcttattacaaaatataaaatataaaatataaaataaaatatacaatccaatactcacataatccaactcactcgaacactatttttccaattacgataacaatattgcagacctgcaggtagtaacccttaattttattaaaataaccgcaatttatttggcggcaacacaggatctctcttttaagttactctctattacatacgttttccatctaaaaattagtagtattgaacttaacggggcatcgtattgtagttttccatattggcgcgcccaaaaaattgaaattttattttttttttttggaatataaataatgccgccaccatgacggccctgacagaaggtgcgaagctgttcgagaaggagattccctatatcacagaattggagggggatgtagagggtatgaagtttatcatcaaaggcgaagggacaggggatgcaacaactggaacaattaaggctaagtacatttgcacgaccggcgacgtcccggtgccctggtccacgctcgtcaccacgctcacgtacggagcccagtgctttgccaaatatggccctgaacttaaagacttctacaagtcgtgtatgccggagggatacgtgcaagagaggacgatcacctttgaaggtgacggagtattcaaaacaagagcggaggtgacgttcgagaatggatcggtctataaccgggtcaagctcaacggacagggctttaagaaagatggacacgtccttgggaagaatttggagttcaatttcaccccgcattgtctttacatctggggtgatcaggcgaatcacgggttgaaatcagcgttcaagatcatgcacgagattacggggagcaaagaggactttatcgtggcagaccacactcagatgaacactccaatcggagggggtcccgtacacgtacccgagtatcatcacctgaccgtctggacatcgtttggaaaagaccctgacgacgatgaaactgatcatctcaacattgtggaagtgatcaaggcggtggacttggaaacataccgcgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatttttatggcgcgcctagtaacccttaattttattaaaataaccgcaatttatttggcggcaacacaggatctctcttttaagttactctctattacatacgttttccatctaaaaattagtagtattgaacttaacggggcatcgtattgtagttttccatattttatccaaacccacccgctttttatagtaagtttttcacccataaataataaatacaataattaatttctcgtaaaagtagaaaatatattctaatttattgcacggtccctatcagtgatagagagccgccaccatgtttgttttccttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttctgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttgaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatttttattaaatttaaatgcgatcgcttgttatttttataaaaacaagaacggtacggcgatatttatttttttctaaaacatctaaccgaagtagtggtatgataaaaatgtagtgtaattgttatatagtgtaacacgaatgcagtttgggagtattcgttgtgctacaaatatatttccattcttcgtggcatttttagctataaaattatatggggttgtctggtttaatatttcggattctcctacccaatagcctcgtctaaggcttcttaaaaaacttaactcgtttggagtctctatcttaattagatccgaatttggatttagagctttgcatgtattacgtccttcctcccaggtttttcgatcagtagataaatggatacatttattattatagcttatccagtcagtaggacaatcttctctaatatatgcatcttttatcgttttatctattgatggtgtatgaactaatttacgttctacaacttttaatagtgtcgctgtaaaaataattcgcgatcgcgcggccgc

SEQ ID NO: 3 - DNA, 4732 bp, synthetic construct
  misc_feature 1..4732: note = EXPRESSION CASSETTE
  source 1..4732: mol_type = other DNA
gcggccgccctgcaggccatcaaaaatataagtgcggtatgagttctcgctataacgatcggagatacaggggcgtacccagtaagcaactccatagcatacgtgttctcatccattagagagttagtatatacaatacaatatttctgaccaataagaggttttagaatattacgattagtgatgagaggaataatttcacccggattaatcttggcaaatgttctgttaggaatagcagctagaacattctttgtagtcttgtaatcaacgatggcggcatcctcgaacttattattttttgagatatcctcgataattttatgccattcagccattgttatttattatatatgttttttaataagacatctattggaataaacttgacattagcattttattcttattacaaaatataaaatataaaatataaaataaaatatacaatccaatactcacataatccaactcactcgaacactatttttccaattacgataacaatattgcagacctgcaggtagtaacccttaattttattaaaataaccgcaatttatttggcggcaacacaggatctctcttttaagttactctctattacatacgttttccatctaaaaattagtagtattgaacttaacggggcatcgtattgtagttttccatattggcgcgcccaaaaaattgaaattttattttttttttttggaatataaataatgccgccaccatgacggccctgacagaaggtgcgaagctgttcgagaaggagattccctatatcacagaattggagggggatgtagagggtatgaagtttatcatcaaaggcgaagggacaggggatgcaacaactggaacaattaaggctaagtacatttgcacgaccggcgacgtcccggtgccctggtccacgctcgtcaccacgctcacgtacggagcccagtgctttgccaaatatggccctgaacttaaagacttctacaagtcgtgtatgccggagggatacgtgcaagagaggacgatcacctttgaaggtgacggagtattcaaaacaagagcggaggtgacgttcgagaatggatcggtctataaccgggtcaagctcaacggacagggctttaagaaagatggacacgtccttgggaagaatttggagttcaatttcaccccgcattgtctttacatctggggtgatcaggcgaatcacgggttgaaatcagcgttcaagatcatgcacgagattacggggagcaaagaggactttatcgtggcagaccacactcagatgaacactccaatcggagggggtcccgtacacgtacccgagtatcatcacctgaccgtctggacatcgtttggaaaagaccctgacgacgatgaaactgatcatctcaacattgtggaagtgatcaaggcggtggacttggaaacataccgcgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatttttatggcgcgcctagtaacccttaattttattaaaataaccgcaatttatttggcggcaacacaggatctctcttttaagttactctctattacatacgttttccatctaaaaattagtagtattgaacttaacggggcatcgtattgtagttttccatattttaattaaatttaaaaattgaaattttattttttttttttggaatataaatatccctatcagtgatagagagccgccaccatgtttgttttccttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactcttaatttttaaatgcgatcgcttgttatttttataaaaacaagaacggtacggcgatatttatttttttctaaaacatctaaccgaagtagtggtatgataaaaatgtagtgtaattgttatatagtgtaacacgaatgcagtttgggagtattcgttgtgctacaaatatatttccattcttcgtggcatttttagctataaaattatatggggttgtctggtttaatatttcggattctcctacccaatagcctcgtctaaggcttcttaaaaaacttaactcgtttggagtctctatcttaattagatccgaatttggatttagagctttgcatgtattacgtccttcctcccaggtttttcgatcagtagataaatggatacatttattattatagcttatccagtcagtaggacaatcttctctaatatatgcatcttttatcgttttatctattgatggtgtatgaactaatttacgttctacaacttttaatagtgtcgctgtaaaaataattcgcgatcgcgcggccgc

SEQ ID NO: 4 - DNA, 6353 bp, synthetic construct
  misc_feature 1..6353: note = EXPRESSION CASSETTE
  source 1..6353: mol_type = other DNA
gcggccgccctgcaggtctacttgctcattagaagtataaaaaaatagttccgtaattaaatggctaagcgagtaagccttccagatgtggttatttcagcacctaaagcagtctttaagcccgctaaagaagaagcactcgcttgtatactaccaaagtattataaatctatggcagatgtgtctattaagacaaatagtgtaattgataagtgttggttttgtaatcaagatttggtttttagacctattagtattgagacatacaagggtggtgaagtagggtatttctgttctaaaatatgtagggattcgttggcttctatggttaagtctcacgtagctcttagagaagaaccaaaaatttctttgttgcctttagtattctatgaagataaggaaaaggttataaatacaataaacttactaagagataaagacggcgtttacggaagctgttactttaaggaaaactcacaaattatagatatttctctacggagtttattgtaagctcctgcaggaatattgccagactggcctttaatcagccgctgatccttgaacccgcctatccgcgggttttcttttgtgcgcttgcaggccagcttgggatcagcagcctgacggatccggtgtccggcgacagcctgactgcccaggaggcactcgcgggcgcgccacactaaattattttttattaataattgtacaagtttttgatctggtataaatacattcaaaaatgataatttaatgacattagttgtgcgggtgtatagagttcacagtagctcattcacttctattcagtcaaaatgtttgattatctggaaaatgaggaggtggctctcgatgaacttaaacagatgttgagagatagagatcctaatgataccaggaaccaattcaagaataatgctctacatgcataccttttcaatgagcactgtaataatgttgaggttgtcaaactactactagacagtggtactaatccattacacaaaaattggagacagcttactccattaggggaatacacaaatagtagacatggtaaagttaataaggatatagcgatggttctactagaagctactggatatagcaacataaatgactttaatatattcacctatatgaaatccaaaaatgtagatattgacttgataaaggtattggtagaacatggatttgattttagtgttaaatgcgaaaaacatcattcagttatagaaaattatgtaatgacagatgatcctgttcctgaaattattgatttattcatagaaaatggatgcagtgttatttatgaggacgaggatgatgagtacggatacgcgtatgaagaatatcactcacaaaatgacgattatcaaccacgaaattgcggtacagtattacatctgtatatcatctctcatctgtattcagagtcggattcgagatcatgtgtgaacccggaagttgttaaatgtctgattaatcatggaatcaacccatcttctatagataaaaactattgtacagctcttcaatattatattaagtcatctcatatagatatagacatcgttaaattgttaatgaaaggaatagataacacggcttattcatatatagacgatctaacatgttgtactcgaggaattatggctgattatctaaatagtgattatagatacaataaagatgtagatttagatttggtcaaattgtttttggagaatggaaaaccgcacggaataatgtgtagtattgtaccactatggagaaatgataaggaaaccatctctttgatattgaaaacaatgaactcggatgtcctccaacatatactaattgagtatataacattcagcgatatcgatatctctctagtggaatacatgttggaatatggagctgtggtaaataaagaggctattcacggatactttaaaaatattaatattgattcttacacgatgaaatatctactaaaaaaggaagggggagatgccgtcaatcatctcgatgatggagagatcccgattggacacctatgtaaatccaactatggacgttataatttctacactgatacatacagacagggttttcgtgatatgtcttatgcttgcccaattcttagtactataaacatttgcctaccttatcttaaagacattaacatgattgacaaacgaggagaaacacttcttcacaaggctgttagatataataaacaatctctagtgtctttactgctagaatccggttcagatgtcaacattagatcaaataacggatatacatgtatagccattgcaatcaacgaatctagaaacattgaactgctgaacatgctattatgtcataaacctacattagattgtgtgattgattcattgagagaaatatctaacatagtagataatgcctatgctataaaacaatgtattagatatgccatgattatagatgactgtatatcgtctaagattccagagtccataagtaaacactataatgattatatagatatttgcaatcaagaattgaacgagatgaaaaaaataatagtgggaggcaacactatgttctcattaatatttactgatcatggagctaaaattattcatcggtatgccaataatccagaattacgtgcgtattatgagtcaaaacaaaataaaatatacgtggaagtatatgatattatttccaatgcgatagtgaagcataataaaattcataaaaacatagaatcagttgatgacaatacctacatttctaatttgccttataccatcaaatacaaaatattcgagcaacaataagtattttttatcaaaaaattgaaattttattttttttttttggaatataaataatatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctcactgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgaacgcgccgaggcccgccaccacctgttccagtagtttttatggcgcgccaatattgccagactggcctttaatcagccgctgatccttgaacccgcctatccgcgggttttcttttgtgcgcttgcaggccagcttgggatcagcagcctgacggatccggtgtccggcgacagcctgactgcccaggaggcactcgcgttaattaaataaaaattaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttctttttgtcctttttaggctctgttggtgggaatgttttgtatgcgtcaatatgcttattcagcaaaatgacttgatctttgaaatttggatctttgtcatccaatttgatggcagctgtgtaggtcaaccacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttggggccgacgttgttttgatcgcgccccactgcgttctccattctggttactgccagttgaatctgagggtccaccaaacgtaatgcggggtgcatttcgctgattttggggtccattatcagacatggtggcggctctctatcactgatagggatatttatattccaaaaaaaaaaaataaaatttcaatttttataaaaattactgtacaagcaaagcaatattgtcactgctactggaatggtctgtgtttaatttatagttgccaatcctgtagcgactgtatgcagcaaaacctgagtcacctgctacacgctgcgaagctcccaatttgtaataagaaagcgttcgtgatgtagcaacagtgatttctttaggcaggtccttgatgtcacagcgtcctagatggtgtccagcaatacgaagatgtccacgaaggatcacagctccgattacgagttcactttctagaagcggtctggtcagaatagtgccatggagtggcacgttgagaagaatgttagtttctggattgaatgaccacatggaacgcgtacgcgcaaacagtctgaaagaagcaatgaagtagctgagccacatcaagcctacaagacaagccattgcgatagcaattccaccggtgatccaatttattctgtaaacagcagcaagcacaaaacaagctaaagttactggccataacagccagaggaaaattaacttaattatatacaagaacctattcctgttggcataggcaaattgtagaagacaaatccatgtaaggaataggaaacctattactaggttccattgttcaaggagctttttaagctcttcaacggtaatagtaccgttggaatctgccatggtggcggctctctatcactgatagggattattatcgataattacaattcaattttaggatacatttaaatttaattaaatttaaatgcgatcgctaatgacctgtataaggtatcttacgatcaaggggtaagtattaccaagattatgggagataataactaataataatgaaaacaaactatagagttgtaaatggatgaaattgtaaaaaatatccgggagggaacgcatgtccttcttccattttatgaaacattgccagaacttaatctgtctctaggtaaaagcccattacctagtctggaatacggagctaattactttcttcagatttctagagttaatgatctaaatagaatgccgaccgacatgttaaaactttttacacatgatatcatgttaccagaaagcgatctagataaagtctatgaaattttaaagattaatagcgtaaagtattatgggaggagtactaaagcggacgccgtagttgccgacctcagcgcacgcaataaactgttcaaacgtgaacgagatgctattaaatctaataatcatctcactgaaaacaatctatacattagcgattatagcgatcgcgcggccgc

SEQ ID NO: 5 - DNA, 4581 bp, synthetic construct
  misc_feature 1..4581: note = EXPRESSION CASSETTE
  source 1..4581: mol_type = other DNA
gcggccgccctgcaggccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcataacctgcaggaatagtttcaaaagaaagctctgaatcaacggactgcgataataagtggtggtatccagaatttgtcacttcaagtaaaaacacctcacgagttaaaacacctaagttctcaccgaatttctcaatatccggacggataatatttattgggcgcgcccaaaaaattgaaattttattttttttttttggaatataaataatatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatttttatggcgcgccaatagtttcaaaagaaagctctgaatcaacggactgcgataataagtggtggtatccagaatttgtcacttcaagtaaaaacacctcacgagttaaaacacctaagttctcaccgaatttctcaatatccggacggataatatttattgttaattaaatttgtatcctaaaattgaattgtaattatcgataataatccctatcagtgatagagagccgccaccatggcagattccaacggtactattaccgttgaagagcttaaaaagctccttgaacaatggaacctagtaataggtttcctattccttacatggatttgtcttctacaatttgcctatgccaacaggaataggttcttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaatttttataaaaattgaaattttattttttttttttggaatataaatatccctatcagtgatagagagccgccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacagctgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaatttttataaatgcgatcgctttttcttattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatcggctcctggtacacatatacgttatttgagagatcatttctataatttaggaatgattatcaaatggatgctaattgacggacgccatcatgatcctattctaaatggattgcgtgatgtgactctagtgactcggttcgttgatgaggaatatctacgatccatcaaaaaacaactgcatccttctaagattattttaatttctgatgtaagatccaaacgaggaggagcgatcgcgcggccgc

SEQ ID NO: 6 - DNA, 3354 bp, synthetic construct
  misc_feature 1..3354: note = EXPRESSION CASSETTE
  source 1..3354: mol_type = other DNA
gcggccgccctgcagggcgcgtcggtattcgtaaacgatgatatatatgtaatgggtggagtttataccacgtatgaaaaatatgtagtattaaacgatgtggaatgtttcactaaaaatcgttggataaaaaagtctcccatgcctagacatcatagtatagtttatgctgtagagtacgacggcgacatctatgtaattactggaattactcacgagactcgtaattatctatacaaatatatagttaaggaagacaaatggatagaattgtacatgtactttaaccatgtaggaaagatgttcgtgtgttcttgcggtgattatatcttaattatagcagatgcaaaatacgaatattatccaaaatcaaatacttggaatttgttcgatatgtcaactcgtaatatcgaatattatgatatgtttactaaagatgagactccaaagtgtaacgtaactcataagtcactgccatcgtttttgagcaactgtgaaaaacaatttctacaatagcctgcaggccaattacctgaagtctttaatctataattggaattgtatctattggtttattggagtagatccttgcttttctgagcaatagctctgatatccaaatcaagccaaaggaatttgttattttggctctgtcagctgaataacgccaaaaaggcgcgcccaaaaaattgaaattttattttttttttttggaatataaataatatgagcagcggcgccctgctgttccacggcaagatcccctacgtggtggagatggagggcaatgtggatggccacaccttcagcatccgcggcaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaaaatctgccacgagatcaccggcagcaagggcgatttcatcgtggccgatcacacccagatgaatacccccatcggcggcggccccgtgcacgtgcccgagtaccaccacatgagctaccacgtgaagctgagcaaggatgtgaccgatcaccgcgataatatgagcctgaaggagaccgtgcgcgccgtggattgccgcaagacctacgacttcgacgccggttccggagatacaagctaatttttatgtttaaacatttagtatcctaaaattgaattgtaattatggataataaatggccaagcctttgtctcaagaagaatccaccctcattgaaagagcaacggctacaatcaacagcatccccatctctgaagattacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaatttttatgtttaaacggcgcgccccaattacctgaagtctttaatctataattggaattgtatctattggtttattggagtagatccttgcttttctgagcaatagctctgatatccaaatcaagccaaaggaatttgttattttggctctgtcagctgaataacgccaaaaattaaaaattgaattgtaattatcgataataatccctatcagtgatagagagccgccaccatggcagattccaacggtactattaccgttgaagagcttaaaaagctccttgaacaatggaacctagtaataggtttcctattccttacatggatttgtcttctacaatttgcctatgccaacaggaataggttcttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaatttttataaatttaaatgcgatcgcatggaagccggaacttccaaaatgtgtacgctaataaacataaaacataaaaattatacaatggtttatgtcagctactttgtgtccgtagtgatataaaatggatactattaaaatatttaatcatggagagtttgatactattagaaatgaattggtcaacttgttaaaagttgtaaaatggaacaccattaattcaaatgtaacggtatcgtctaccgacacaatagatatatctgattgtatcagagagatattatacaaacagtttaaaaatgtacgaaatatagaggtgagcagtgatatatctttcataaaatataatagatttaatgatactactctaacagatgataatgtgggatattatttagtgatttatttgaatcggacgaaatctgtaaagactttaatatatcctactccagaaacagtgataacatcatccgaggatataatgttttctaaatctcttaactttagattcgagaatgtaaagcgcgatcgcgcggccgc

SEQ ID NO: 7 - DNA, 5596 bp, synthetic construct
  misc_feature 1..5596: note = EXPRESSION CASSETTE
  source 1..5596: mol_type = other DNA
gcggccgccctgcaggtctacttgctcattagaagtataaaaaaatagttccgtaattaaatggctaagcgagtaagccttccagatgtggttatttcagcacctaaagcagtctttaagcccgctaaagaagaagcactcgcttgtatactaccaaagtattataaatctatggcagatgtgtctattaagacaaatagtgtaattgataagtgttggttttgtaatcaagatttggtttttagacctattagtattgagacatacaagggtggtgaagtagggtatttctgttctaaaatatgtagggattcgttggcttctatggttaagtctcacgtagctcttagagaagaaccaaaaatttctttgttgcctttagtattctatgaagataaggaaaaggttataaatacaataaacttactaagagataaagacggcgtttacggaagctgttactttaaggaaaactcacaaattatagatatttctctacggagtttattgtaagctcctgcaggaatattgccagactggcctttaatcagccgctgatccttgaacccgcctatccgcgggttttcttttgtgcgcttgcaggccagcttgggatcagcagcctgacggatccggtgtccggcgacagcctgactgcccaggaggcactcgcgggcgcgccacactaaattattttttattaataattgtacaagtttttgatctggtataaatacattcaaaaatgataatttaatgacattagttgtgcgggtgtatagagttcacagtagctcattcacttctattcagtcaaaatgtttgattatctggaaaatgaggaggtggctctcgatgaacttaaacagatgttgagagatagagatcctaatgataccaggaaccaattcaagaataatgctctacatgcataccttttcaatgagcactgtaataatgttgaggttgtcaaactactactagacagtggtactaatccattacacaaaaattggagacagcttactccattaggggaatacacaaatagtagacatggtaaagttaataaggatatagcgatggttctactagaagctactggatatagcaacataaatgactttaatatattcacctatatgaaatccaaaaatgtagatattgacttgataaaggtattggtagaacatggatttgattttagtgttaaatgcgaaaaacatcattcagttatagaaaattatgtaatgacagatgatcctgttcctgaaattattgatttattcatagaaaatggatgcagtgttatttatgaggacgaggatgatgagtacggatacgcgtatgaagaatatcactcacaaaatgacgattatcaaccacgaaattgcggtacagtattacatctgtatatcatctctcatctgtattcagagtcggattcgagatcatgtgtgaacccggaagttgttaaatgtctgattaatcatggaatcaacccatcttctatagataaaaactattgtacagctcttcaatattatattaagtcatctcatatagatatagacatcgttaaattgttaatgaaaggaatagataacacggcttattcatatatagacgatctaacatgttgtactcgaggaattatggctgattatctaaatagtgattatagatacaataaagatgtagatttagatttggtcaaattgtttttggagaatggaaaaccgcacggaataatgtgtagtattgtaccactatggagaaatgataaggaaaccatctctttgatattgaaaacaatgaactcggatgtcctccaacatatactaattgagtatataacattcagcgatatcgatatctctctagtggaatacatgttggaatatggagctgtggtaaataaagaggctattcacggatactttaaaaatattaatattgattcttacacgatgaaatatctactaaaaaaggaagggggagatgccgtcaatcatctcgatgatggagagatcccgattggacacctatgtaaatccaactatggacgttataatttctacactgatacatacagacagggttttcgtgatatgtcttatgcttgcccaattcttagtactataaacatttgcctaccttatcttaaagacattaacatgattgacaaacgaggagaaacacttcttcacaaggctgttagatataataaacaatctctagtgtctttactgctagaatccggttcagatgtcaacattagatcaaataacggatatacatgtatagccattgcaatcaacgaatctagaaacattgaactgctgaacatgctattatgtcataaacctacattagattgtgtgattgattcattgagagaaatatctaacatagtagataatgcctatgctataaaacaatgtattagatatgccatgattatagatgactgtatatcgtctaagattccagagtccataagtaaacactataatgattatatagatatttgcaatcaagaattgaacgagatgaaaaaaataatagtgggaggcaacactatgttctcattaatatttactgatcatggagctaaaattattcatcggtatgccaataatccagaattacgtgcgtattatgagtcaaaacaaaataaaatatacgtggaagtatatgatattatttccaatgcgatagtgaagcataataaaattcataaaaacatagaatcagttgatgacaatacctacatttctaatttgccttataccatcaaatacaaaatattcgagcaacaataagtattttttatcaaaaaattgaaattttattttttttttttggaatataaataatatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctcactgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgaacgcgccgaggcccgccaccacctgttccagtagtttttatggcgcgccaatattgccagactggcctttaatcagccgctgatccttgaacccgcctatccgcgggttttcttttgtgcgcttgcaggccagcttgggatcagcagcctgacggatccggtgtccggcgacagcctgactgcccaggaggcactcgcgttaaaaaattgaaattttattttttttttttggaatataaatatccctatcagtgatagagagccgccaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacagctgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaataatttttatatttaaatgcgatcgctaatgacctgtataaggtatcttacgatcaaggggtaagtattaccaagattatgggagataataactaataataatgaaaacaaactatagagttgtaaatggatgaaattgtaaaaaatatccgggagggaacgcatgtccttcttccattttatgaaacattgccagaacttaatctgtctctaggtaaaagcccattacctagtctggaatacggagctaattactttcttcagatttctagagttaatgatctaaatagaatgccgaccgacatgttaaaactttttacacatgatatcatgttaccagaaagcgatctagataaagtctatgaaattttaaagattaatagcgtaaagtattatgggaggagtactaaagcggacgccgtagttgccgacctcagcgcacgcaataaactgttcaaacgtgaacgagatgctattaaatctaataatcatctcactgaaaacaatctatacattagcgattatagcgatcgcgcggccgc

SEQ ID NO: 8 - DNA, 2803 bp, synthetic construct
  misc_feature 1..2803: note = EXPRESSION CASSETTE
  source 1..2803: mol_type = other DNA
gcggccgccctgcaggtctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattacctgcagggctcgccagttccaaaacgtgtcggcggagcagattgcgtatcttgctcagttgcagcgttccggcgatcaagccggggcaatccaggcggcgaacgaggccgcaacgaaagggtttgatcaccagacccgccgcctgaaagagaacaacggcgcgcccaaaaaattgaaattttattttttttttttggaatataaataatatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaatgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatttttatggcgcgccgctcgccagttccaaaacgtgtcggcggagcagattgcgtatcttgctcagttgcagcgttccggcgatcaagccggggcaatccaggcggcgaacgaggccgcaacgaaagggtttgatcaccagacccgccgcctgaaagagaacaacttaaaaaattgaaattttattttttttttttggaatataaatatccctatcagtgatagagagccgccaccatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaatttttattaaatttaaatgcgatcgcctaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacgatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatgcgatcgcgcggccgc

SEQ ID NO: 9 - protein, 8 aa, CORONAVIRUS
YNYLYRLF

SEQ ID NO: 10 - protein, 8 aa, CORONAVIRUS
VVLSFELL

SEQ ID NO: 11 - protein, 8 aa, CORONAVIRUS
VNFNFNGL

(19) United States
(12) Patent Application Publication
(10) Pub. No.: US 2023/0381297 A1
(43) Pub. Date: Nov. 30, 2023

(54) ATTENUATED POXVIRUS VECTOR BASED VACCINE FOR PROTECTION AGAINST COVID-19

(71) Applicant: SEMENTIS LIMITED, Hackney (AU)

(72) Inventors: Natalie PROW, Grange (AU); Paul Howley, Berwick (AU); Tamara Cooper, Klemzig (AU); John D. Hayball, Sellicks Hill (AU); Kerrilyn R. Diener, Sellicks Hill (AU); Liang Liu, St. Peters (AU); Preethi Eldi, Belair (AU)

(21) Appl. No.: 18/317,386

(22) Filed: May 15, 2023

Related U.S. Application Data
(63) Continuation of application No. 17/212,327, filed on Mar. 25, 2021, now abandoned.
(60) Provisional application No. 63/003,012, filed on Mar. 31, 2020.
(60) Provisional application No. 63/066,927, filed on Aug. 18, 2020.

(51) Int. Cl. A61K 39/215; C12N 15/86

(52) U.S. Cl. CPC A61K 39/215; C12N 15/86; C12N 2710/24143; C12N 2710/24134; A61K 2039/5254

(57) ABSTRACT

The present invention relates to a composition for raising an immune response in an animal which prevents or decreases the risk of a coronavirus infection and decreases severity of disease. In particular, the invention relates to vaccines and/or immunogenic compositions for raising an immune response in an animal which prevents or decreases the risk of the SARS-CoV-2 disease named COVID-19 by the World Health Organization. The composition comprises an attenuated poxvirus, and especially a vaccinia virus, wherein the attenuated poxvirus genome comprises a coronavirus SARS-CoV-2 nucleic acid sequence encoding the spike protein polypeptide and or the membrane protein polypeptide and or nucleocapsid protein polypeptide and or envelope protein polypeptide or an immunogenic or functional part of any of these.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 57,344 Bytes ASCII (Text) file named “SEQUENCE_LISTING.txt,” created on 18 Mar. 2021.

### FIELD OF THE INVENTION

The present invention relates to a composition for raising an immune response in an animal which prevents or decreases the risk of a coronavirus infection and decreases severity of disease. In particular, the invention relates to vaccines and/or immunogenic compositions for raising an immune response in an animal which prevents or decreases the risk of the SARS-CoV-2 disease named COVID-19 by the World Health Organization. The composition comprises an attenuated poxvirus, and especially a vaccinia virus, wherein the attenuated poxvirus genome comprises a coronavirus SARS-CoV-2 nucleic acid sequence encoding the spike protein polypeptide and or the membrane protein polypeptide and or nucleocapsid protein polypeptide and or envelope protein polypeptide or an immunogenic or functional part of any of these.

### BACKGROUND

Bibliographic details of references in the subject specification are listed at the end of the specification.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavor to which this specification relates.

All publications mentioned in this specification are herein incorporated by reference in their entirety.

The poxvirus family comprises two subfamilies, the Chordopoxvirinae and Entomopoxvirinae. The Chordopoxvirinae comprises eight genera including the Orthopoxviridae comprising species which infect man (for example, variola virus, the causative agent of smallpox, cowpox virus (which formed the original smallpox vaccine reported by Jenner in 1796), vaccinia virus (used as a second-generation smallpox vaccine) and monkeypox virus), and the Avipoxviridae viruses comprising species that infect birds, such as fowlpox and canarypox viruses. In addition to their use as antigens in smallpox vaccines, there is much interest in the use of recombinant vaccinia-based viruses and avipox viruses as “backbone” vectors. As intra-cytoplasmic vectors, the Orthopoxviridae are able inter alia to induce production of foreign antigens in the host cytoplasm for recognition by the host immune system. Such vectors expressing foreign antigens are intensively researched in the development of vaccines for diseases such as AIDS, tuberculosis, malaria, and cancer which have proven difficult to treat by other vaccination strategies.

The Chordopoxvirinae have linear double-stranded DNA genomes ranging in size from 130 kb in parapoxviruses to over 300 kb in avipoxviruses and their life cycle in the host is spent entirely in the host cell cytoplasm. The poxviruses operate mostly independently of their host cell and host cell molecules, especially for processes involved in early mRNA synthesis. However, host molecules appear to be used for the initiation or termination of intermediate and late viral transcription. The poxviruses produce structurally diverse “host range factors” which specifically target and manipulate host signaling pathways to permit cellular conditions allowing viral replication. Most poxviruses can bind and infect mammalian cells, but whether or not the subsequent infection is permissive (able to produce infectious virions) or non-permissive (substantially unable to produce infections virions) is dependent upon the specific poxvirus and specific cell type involved. For a review of host range genes, refer to Werden et al. 2008 incorporated herein in its entirety.

Strains of vaccinia relevant to their use as smallpox vaccines and subsequently as viral vectors, have been published from the early 1960's through to the present day. Certain strains of vaccinia, including strains employed as smallpox vaccines, are able to propagate in human cells and therefore represent health risks, such as the development of viral encephalitis. With a view to developing a safer vaccine, a vaccinia strain from Ankara (referred to as “CVA”) was passaged more than 500 times in non-human cells. During this process, the vaccinia genome changed substantially involving the development of at least six major deletions compared to the original CVA genome. The modified virus was less pathogenic, due to replication deficiencies in mammalian cells, but still able to engender a protective immune response. This attenuated vaccinia virus is referred to as MVA (Modified Vaccinia Ankara) and is also categorized by passage number, as viruses with different passage numbers were found to be genetically and phenotypically distinct. However, by passage number 515, MVA515, was determined to be genetically stable. In the early 1990s, it was observed that MVA strains, such as MVA572, and its derivative, MVA F6 were able to express vaccinia proteins and heterologous (recombinant) proteins at high levels in non-permissive cells (in which the virus will not propagate), enabling the development of MVA as a vector for heterologous molecules of interest, such as those encoding antigens for vaccine or therapy delivery. MVA is the most studied among the poxviral vaccine vector systems but other poxviruses have been developed to function in a similar way such as NYVAC, ALVAC, and fowlpox.

Another vaccinia virus, which is developed by Sementis Ltd, is the so-called SCV (Sementis Copenhagen Vaccinia) vaccinia virus vector. The SCV has been generated using the Copenhagen strain of vaccinia and engineered by deletion of D13L, which encodes an essential viral assembly protein, thereby rendering SCV unable to replicate and produce infectious progeny. Genome amplification is preserved in SCV-infected cells, thus permitting late-phase expression of vaccine antigens and a generation of a strong immune response against the inserted antigens. SCV possesses a number of advantages compared to MVA in that it possesses the immunogenicity of replication competent vaccinia and is unable to replicate in mammalian cells tested. The SCV platform has two key points of difference from the MVA platform, namely (i) it has been specifically engineered to be replication-deficient through the targeted deletion of the D13L gene, thus safer, whilst maintaining potency with single-shot efficacy and (ii) it is also designed to be manufactured in a standard and scalable commercial cell-line.

Coronaviruses are RNA viruses consisting of a positive-sense, single-stranded RNA of approximately 27-32 kilobases. As the name indicates, the spherical external spike protein displays a characteristic crown shape when observed under an electron microscope. The virus is known to infect a wide range of hosts, including humans. Infected hosts exhibit different clinical courses ranging from asymptomatic to severe symptoms. Coronaviruses belong to the Coronaviridae family, which are divided into four genera: Alpha-, Beta-, Delta-, and Gamma-coronaviruses. CoVs are commonly found in many species of animals, including bats, camels, and humans. Occasionally, the animal CoVs can acquire genetic mutations by errors during genome replication or recombination mechanisms, which can further expand their tropism to humans. The first human CoVs were discovered in the mid-1960s. A total of seven human CoV types were identified to be responsible for causing human respiratory ailments, which include two alpha CoVs and five beta CoVs. Typically, these CoVs can cause a range of clinical symptoms ranging from asymptomatic infection to severe acute respiratory illness, including fever, cough, and shortness of breath. Other symptoms such as gastroenteritis and neurological diseases of varying severity have also been reported.

Coronaviruses contain a canonical set of four major structural proteins: Spike (S), membrane (M), envelope (E) protein, and the nucleocapsid (N) protein. The virion possesses a nucleocapsid composed of genomic RNA and the phosphorylated nucleocapsid (N) protein. The nucleocapsid is buried inside phospholipid bilayers and covered by spike (S) proteins. The membrane (M) protein and the envelope (E) protein are located among the S proteins in the viral envelope.

The spike protein is composed of a transmembrane trimetric glycoprotein protruding from the viral surface, which determines the diversity of coronaviruses and host tropism. Spike comprises two functional subunits; 51 subunit, which contains the receptor-binding domain (RBD) and is responsible for binding to the host cell receptor and S2 subunit for the fusion of the viral and cellular membranes.

Coronavirus particles consist of a helical nucleocapsid structure, formed by the interaction of the nucleocapsid phosphoproteins and the viral genomic RNA, which is surrounded by a lipid bilayer where the structural proteins are inserted. The triple-spanning membrane glycoprotein M drives the assembly of coronaviruses, which bud into the lumen of the endoplasmic reticulum-Golgi intermediary compartment (ERGIC). Membrane protein is the most abundant viral protein that sorts viral components to be incorporated into virions. Membrane oligomerization allows the formation of a lattice of membrane proteins at the ERGIC membranes. Spike and envelope proteins are integrated into the lattice through lateral interactions with membrane protein, whereas nucleocapsid and viral RNA interact with the membrane C-terminal domain, which is exposed to the cytosol. The envelope protein is a viroporin that forms ion channels and plays an important role in virus morphogenesis and budding, however this process is not fully understood to date. Studies on SARS-CoV have demonstrated that depletion of the envelope gene from coronavirus genome strongly diminish virus growth and particle formation. The nucleocapsid protein self-associates and encapsidates the RNA genome for incorporation within the virion.

Human coronaviruses are one of the main pathogens causing respiratory infection. The two highly pathogenic viruses, SARS-CoV and MERS-CoV, cause severe respiratory syndrome in humans and four other human coronaviruses (HCoV-OC43, HCoV-229E, HCoV-NL63, HCoVHKU1) induce mild upper respiratory disease. The SARS-CoV caused a major outbreak involving 8422 patients during 2002-03 and spread to 29 countries globally. The epidemic was contained in July 2003 as the transmission chain of SARS-CoV in Taiwan was interrupted and no more human cases have been reported since May 2004. MERS-CoV emerged in Middle Eastern countries in 2012 and has been causing persistent endemics in countries within and sporadically spreading to countries outside the Middle East regions. In late 2019, the first reports of an unknown respiratory infection emerged from Wuhan, China. The infection source was quickly identified as a novel coronavirus, called SARS-CoV-2 and the disease caused by this virus named COVID-19. The World Health Organization declared a pandemic on Mar. 11, 2020. SARS-CoV-2 has an infection fatality rate ranging from 0.16% to 1.60% and by mid-February 2021, SARS-CoV-2 has infected 108.2 million people and caused 2.3 million deaths globally. SARS-CoV-2 has forced much of the world to adopt a lockdown practice which has resulted in staggering economic fallout and human suffering.

SARS-CoV-2 is an enveloped, non-segmented, positive sense RNA virus that is included in the Orthocoronavirinae subfamily which is broadly distributed in humans and other mammals. As part of the sarbecovirus genus, its diameter is about 65 to 125 nm and contains single stranded RNA, with club-shaped glycoprotein spikes on the outer surface giving the virus a crown-like or coronal appearance. SARS-CoV-2 is a novel β-coronavirus after the previously identified SARS-CoV and MERS-CoV which led to pulmonary failure and potentially fatal respiratory tract infection. The reproductive number of SARS-CoV-2, which describes the capability of transmission per primary infected person to the secondarily infected persons, is estimated to range between 1.4 to 2.5 by the WHO. However meta-analysis of global studies estimate the reproductive number closer to 2.87. The reproductive number of SARS-CoV-2 is considerably higher than the previous infectious coronaviruses such as SARS-CoV and MERS respectively (0.95 and 0.91). The possible reasons for the higher reproductive number may be the inherent biological features to this virus strain. For instance, a person could be infected by numerous ways, such as close physical contact with the infected person, through environmental transmission by respiratory droplets, fomites, and airborne transmission. Moreover, SARS-CoV-2 infected patients may not show symptomatic characteristics up to two weeks of infection which may increase the risk of new infections exponentially as the infected person is usually confounded in the community with other people during the asymptomatic stage. Phylogenetic analysis from nine patients' samples showed that SARS-CoV-2 was more similar to two bat-derived coronavirus strains than to known human-infecting coronaviruses, including the virus that caused the SARS outbreak of 2003. Sequences from different patients were almost identical with greater than 99.9% sequence identity suggesting that SARS-CoV-2 originated from a single source within a very short period. Currently available data suggest that SARS-CoV-2 infected the human population from a bat reservoir and that SARS-CoV-2 might have evolved from the bat coronavirus by accumulating favorable genetic changes for human infection. The structural proteins spike and membrane were shown to have extensive mutational changes, whereas envelope and nucleocapsid proteins were conserved. It remains unclear if a currently unknown animal species acts as an intermediate host between bats and humans.

Structural and functional analysis showed that the RBD of the SARS-CoV-2 strongly interacts with the human Angiotensin-Converting Enzyme 2 (ACE2) receptor. ACE2 expression was highest in lung, heart, ileum, kidney and bladder. In lung, ACE2 was highly expressed on lung epithelial cells. Following binding of SARS-CoV-2 to the ACE2 receptor, the spike protein undergoes protease cleavage. A two-step sequential protease cleavage to activate spike protein of SARS-CoV and MERS-CoV was proposed as a model, consisting of cleavage at the S1/S2 cleavage site for priming and a cleavage for activation at the S′2 site, a position adjacent to a fusion peptide within the S2 subunit. After cleavage at the S1/S2 cleavage site, 51 and S2 subunits remain non-covalently bound and the distal 51 subunit contributes to the stabilization of the membrane-anchored S2 subunit in the prefusion state. Subsequent cleavage at the S′2 site presumably activates the spike for membrane fusion via irreversible, conformational changes. The coronavirus spike is unique among viruses because a range of different proteases can cleave and activate it. The characteristics specific to SARS-CoV-2 among coronaviruses is the existence of a furin cleavage site (“RPPA” sequence) at the S1/S2 site. The S1/S2 site of SARS-CoV-2 was entirely subjected to cleavage during biosynthesis in contrast to SARS-CoV spike, which was incorporated into assembly without cleavage. Although the S1/S2 site was also subjected to cleavage by other proteases such as transmembrane protease serine 2 (TMPRSS2) and cathepsin L, the ubiquitous expression of furin also likely contributes to more efficient viral replication leading to increased virulence. In SARS-CoV-2, the viral spike protein and in particular the RBD is a locus for viral evolution. From October 2020, evolution in the virus identified as amino acid changes within the RBD were detected in Europe, Brazil, United Kingdom, and South Africa. The accumulation of mutations resulted in approximately one mutation occurring every two weeks, as exhibited by the variants emerging from the UK, South Africa, and Brazil which had 8 mutations, 7 mutations, and 10 mutations in the Spike RBD, respectively, as well as the deletion of 3 amino acids in the 1ab open reading frame (ORF). The characteristics of these variant strains of SARS-CoV-2 indicate repeated convergent evolution towards viral species with enhanced fitness. A possible hypothesis for this phenomenon is that these mutations emerge from cases of chronic COVID-19 infection during which the immune system places great pressure on the virus to escape immunity and the virus reacts by refining its mechanisms of infiltrating cells. This therefore translates to increased viral load and higher transmissibility, as demonstrated by the UK mutant strain, B.1.1.7. In a virus-naïve population, natural immunity selection pressure is the main driver for the emergence of variants. However, there is also a possibility of a ‘vaccine selection pressure’ wherein mutations may be conceived due to vaccine-related reasons. The virus could evolve antigenically when vaccines elicit a restricted immune response such as when vaccines are directed to raise an immune response against a single antigen only. Time delays in administering vaccine shots across a whole population can also potentially affect the adaptability of the virus and induce mutations that can help them evade or resist immune response. The phenomenon of escape mutants highlights the need for a vaccine that works effectively against emerging variants of concern (VOC). As most of the recorded mutations are occurring in the spike protein, other viral antigens may be utilized to induce a wider breadth of immunogenicity to ensure adequate prophylactic immune response.

Neutralizing antibodies are antibodies that bind and neutralize the virus within host cells and serve as a key correlate of immunity for prophylactic vaccination. Neutralizing antibodies against SARS and MERS spike glycoproteins play a predominant role in the protection against these coronaviruses. To date, following SARS-CoV-2 infection, it is not known what magnitude of neutralizing antibodies is needed for protection, or what the durability of the neutralizing antibodies would be.

T cell immunity may also be utilized as a correlate of protection against SARS-CoV-2. T cell activation has been reported at both the acute and memory phases of infection, however the exact role of both CD4 and CD8 T cells in disease progression or protection is yet to be fully understood. Antigen-specific CD8 T cells directly target virus-infected cells, while Th1 polarized CD4 T cells have the potential to activate CD8 T cells and monocytes to combat virus-infected cells in tissues. In addition, T follicular helper cells are necessary for germinal center responses and the formation of high quality humoral immune responses. Consistent with this, multiple studies have noted a correlation between binding antibody titres and CD4 T cell response. Thus, T cells can have a protective function both via direct elimination of infected cells, and via activation of other leukocytes and enhancement of humoral immune responses. Moreover, the induction of a robust Th1-biased response is consistent with the unlikely occurrence of vaccine-associated enhanced respiratory disease or antibody-dependent enhancement (ADE) which is associated with a Th2 response.

The vaccines of the instant invention are based on a combination of antigens to generate better long-term protection and guard against the potential for antibody-dependent enhancement of disease. This is a major distinction over vaccines being developed which comprise a spike antigen only. The presence of multiple antigens may also address the phenomenon of escape mutants as multiple antigens may elicit a wider breadth of immune responses, which would make it more difficult for the virus to evolve antigenically and erode the effectiveness of the body's defenses.

The antigenic sequences for the vaccines of the instant invention comprise the spike protein, membrane, nucleocapsid, and envelope proteins of SARS-CoV-2.

Immunogenicity may be achieved by expressing the SARS-CoV-2 spike polypeptide or the S1 receptor binding domain subunit of the spike polypeptide from a poxvirus vector. The spike protein of coronaviruses contains the major neutralizing domains which are essential to neutralize the virus required during the acute phase of viral infection and is required to stimulate cell-mediated immunity. Historically, the spike protein of coronaviruses such as SARS-CoV or MERS-CoV have been found immunogenic, eliciting humoral immune responses including neutralizing antibodies that inhibit virus entry into host cells as well as cell-mediated immune responses.

Immunogenicity may be achieved by expressing the SARS-CoV-2 membrane protein polypeptide from a poxvirus vector. In SARS-CoV, the membrane protein has been shown to be abundant on the viral surface; moreover, when used for immunization in patients with SARS, the membrane protein induced high titres of neutralizing antibodies. Immunogenic and structural analyses demonstrated that a T-cell epitope cluster capable of triggering a robust cellular immune response exists in the membrane protein. As the membrane protein is also highly conserved in many virus species, it is a good antigen candidate for inducing immune response against SARS-CoV-2.

Immunogenicity may be achieved by expressing the SARS-CoV-2 nucleocapsid protein polypeptide from a poxvirus vector. It was recently discovered that SARS-CoV-2 infection leads to production of antibodies that are mostly directed to the nucleocapsid antigen. However, N antibodies have been overlooked as N protein antibodies cannot block virus entry and as such are considered ‘non-neutralizing’ antibodies. Therefore, anti-N antibodies cannot be measured by neutralization assays that are currently in use to assess humoral immunity. Recent studies have shown that anti-N antibodies that get inside cells are recognized by an antibody receptor TRIM21, which then shreds the associated N protein. N protein epitopes are then displayed for detection by T cells. As this immune response mechanism involves T cells that will eventually mediate immunological memory, antibodies against the nucleocapsid protein may stimulate long-term protection against future infection.

Immunogenicity may be achieved by simultaneously expressing the SARS-CoV-2 spike protein, or parts thereof, membrane protein polypeptide, nucleocapsid protein polypeptide, and/or envelope protein polypeptide within a poxvirus vector. Studies on mouse hepatitis virus, bovine coronavirus, infectious bronchitis virus, transmissible gastroenteritis virus, and SARS-CoV has established that spike, membrane, nucleocapsid, and in some cases, envelope structural proteins, are required for the efficient assembly and release of virus like particles (VLPs) by transfected cells. The presence of S, M, and N and/or E polypeptides may lead to the formation of authentic VLPs, empty virus shells that mimic the coronavirus structure but lack the genetic material to be infectious. VLPs share similar size and morphological features with authentic virions but are non-infectious and unable to replicate. VLPs not only mimic the morphology of the native virus but can also transduce permissive cells. Devoid of viral genetic material, VLPs do not replicate within the host cell, but can be used as carriers for nucleic acids, proteins, or drugs. Further, VLPs have been investigated for use as vaccine candidates as their repetitive exposition of surface antigens and their inherent structure can emulate native viruses and interact with the immune system to induce humoral and cellular responses. The combined immunogenicity of these proteins may bring about a more robust antigen-specific immune response. The invention encompasses the use of SARS-CoV-2 spike protein or part thereof, and/or SARS-CoV-2 membrane and/or SARS-CoV-2 nucleocapsid proteins or parts thereof, and/or SARS-CoV-2 envelope protein or part thereof, as antigen/s in a poxviral-vectored vaccine.

The invention encompasses the use of multiple SARS-CoV-2 proteins to elicit a broad range of immune responses, including humoral and cell-mediated immunity. In a natural infection, the immune system recognizes all the proteins comprising SARS-CoV-2, to varying degrees. By utilizing structural genes with less mutational frequencies such as the M, N, and E, in the formulation of a vaccine, the breadth of immune response induced may be expanded to protect against emerging variants of SARS-CoV-2 and reduce the probability of escape mutants.

In an embodiment, the spike protein or part thereof is used as a vaccine antigen in a single poxviral-vectored vaccine.

In an embodiment, the membrane protein or part thereof is used as a vaccine antigen in a single poxviral-vectored vaccine.

In an embodiment, the nucleocapsid protein or part thereof is used as a vaccine antigen in a single poxviral-vectored vaccine.

In an embodiment, the membrane and nucleocapsid proteins or part thereof of any are used as vaccine antigens in a single poxviral-vectored vaccine.

In an embodiment, the spike protein or part thereof, membrane protein or part thereof, and nucleocapsid proteins or part thereof, are used as vaccine antigens in a single poxviral-vectored vaccine.

In an embodiment, the spike protein or part thereof, membrane and nucleocapsid proteins or part thereof of any, and envelope protein or part thereof are used as vaccine antigens, in a single poxviral-vectored vaccine.

In an embodiment, the spike protein or part thereof and membrane and nucleocapsid proteins or part thereof are combined as a mixture of single vaccines.

### SUMMARY

The present inventors have found that by utilizing a poxvirus, and especially a vaccinia virus, attenuated by deletion of at least one gene which encodes an endogenous essential assembly or maturation protein and which has been engineered such that its genome comprises a nucleic acid sequence encoding the spike protein polypeptide and/or the membrane protein polypeptide and/or nucleocapsid protein polypeptide and/or the envelope protein polypeptide of SARS-CoV-2, or an immunogenic or functional part of any thereof, that a composition can be obtained which prevents or decreases the risk of SARS-CoV-2 infection and/or decrease the severity of COVID-19 disease.

Accordingly in a first aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike and/or membrane and/or nucleocapsid and/or envelope polypeptides of SARS-CoV-2 or an immunogenic or functional part or parts of any thereof substituted into open reading frames of selected vaccinia virus genes or inserted into intergenic regions.

In a second aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In a third aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In a fourth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter.

In a fifth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter.

In a sixth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In a seventh aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter.

In an eighth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In a ninth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter, the membrane polypeptide of SARS-CoV-2 or an immunogenic or functional part thereof under transcriptional control of a fowlpox early/late promoter, the nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In a tenth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter, and the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In an eleventh aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter, and the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In a twelfth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the membrane polypeptide of SARS-CoV-2 or an immunogenic or functional part thereof under transcriptional control of a fowlpox early/late promoter.

In a thirteenth aspect the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the nucleocapsid polypeptide of SARS-CoV-2 or an immunogenic or functional part thereof under transcriptional control of a synthetic early/late promoter.

In a fourteenth aspect the present invention provides a method of inducing a protective immune response in a subject against SARS-CoV-2 virus infection the method comprising administering to the subject the composition as any of the above.

In a fifteenth aspect the present invention provides a method of inducing a protective immune response in a subject against SARS-CoV-2 virus infection the method comprising administering to the subject the composition of the fourth and sixth aspect of the present invention.

In a sixteenth aspect the present invention provides a method of inducing a protective immune response in a subject against SARS-CoV-2 virus infection the method comprising administering to the subject the composition of the fifth and sixth aspect of the present invention.

In a seventeenth aspect the present invention provides a composition for raising an immune response in animal which prevents or decreases the risk of SARS-CoV-2 infection and/or decrease the severity of COVID-19 disease by resembling SARS-CoV-2 virus-like particles.

In an eighteenth aspect the present invention provides a composition for raising an immune response in animal which prevents or decreases the risk of SARS-CoV-2 infection and/or decrease the severity of COVID-19 disease and any other infection caused by coronaviruses with genetic similarity to SARS-CoV-2, the composition comprising an attenuated poxvirus, and especially a vaccinia virus, wherein the poxvirus genome comprises a nucleic acid sequence encoding the spike polypeptide of SARS-CoV-2 or an immunogenic or functional part thereof, and/or the membrane and nucleocapsid polypeptides of SARS-CoV-2 or an immunogenic or functional part thereof, and/or the envelope polypeptide of SARS-CoV-2 or an immunogenic or functional part thereof.

In a nineteenth aspect the present invention provides the use of the composition of the first through eighteenth aspects of the present invention in the preparation of a medicament for inducing a neutralizing antibody response and/or protective immune response in a subject against a coronavirus infection.

The invention also includes:

A composition for raising an immune response in an animal which prevents or decreases the risk of SARS-CoV-2 coronavirus disease, the composition comprising a genetically engineered attenuated vaccinia virus, wherein the vaccinia virus genome comprises a nucleic acid sequence encoding at least one human coronavirus SARS-CoV-2 polypeptide selected from the group consisting of a spike protein polypeptide or an immunogenic part thereof, a membrane protein polypeptide or an immunogenic part thereof, a nucleocapsid protein polypeptide or an immunogenic part thereof, and an envelope protein polypeptide or an immunogenic part thereof, wherein the attenuated vaccinia virus comprises a deletion of at least one gene which encodes an endogenous essential assembly or maturation protein, such a
composition wherein the attenuated vaccinia virus genome comprises a nucleic acid sequence encoding a human coronavirus SARS-CoV-2 spike protein polypeptide or an immunogenic part thereof, such a
composition wherein the attenuated vaccinia virus genome comprises a nucleic acid sequence encoding a human coronavirus SARS-CoV-2 membrane protein polypeptide or immunogenic part thereof, such a
composition wherein the attenuated vaccinia virus genome comprises a nucleic acid sequence encoding a human coronavirus SARS-CoV-2 nucleocapsid protein polypeptide or immunogenic part thereof, such a
composition wherein the attenuated vaccinia virus genome comprises a nucleic acid sequence encoding a human coronavirus SARS-CoV-2 membrane protein polypeptide or immunogenic part thereof and nucleocapsid protein polypeptide or immunogenic part thereof, such a
composition wherein the attenuated vaccinia virus genome comprises a nucleic acid sequence encoding a spike protein polypeptide or an immunogenic part thereof, and membrane protein polypeptide or an immunogenic part thereof and nucleocapsid protein polypeptide or immunogenic part thereof, of human coronavirus SARS-CoV-2, such a
composition wherein the attenuated vaccinia virus genome comprises a nucleic acid sequence encoding a human coronavirus SARS-CoV-2 spike polypeptide or immunogenic part thereof, and a membrane protein polypeptide or immunogenic part thereof, and a nucleocapsid protein polypeptide or immunogenic part thereof, and an envelope protein polypeptide or immunogenic part thereof, such a
composition wherein the at least one nucleic acid sequence encoding a human coronavirus SARS-CoV-2 polypeptide is inserted into deleted ORFs of one or more immune modulatory genes selected from the group comprising of COP-C23L, COP-B29R, COP-C3L, COP-N1L, COP-A35R, COP-A39R, COP-A41L, COP-A44R, COP-A46R, COP-B7R, COP-B8R, COP-B13R, COP-B16R, and COP-B19R, such a
composition wherein the at least one nucleic acid sequence encoding a human coronavirus SARS-CoV-2 polypeptide is inserted into an intergenic region (IGR) of the attenuated vaccinia virus genome, wherein the IGR is located between or is flanked by two adjacent ORFs of the vaccinia virus genome, such a
composition wherein the IGR of the attenuated vaccinia virus genome is selected from the group consisting of F9L-F10L, F12L-F13L, F17R-E1 L, E1L-E2L, E8R-E9L, E9L-E10R, I1L-12L, 12L-13L, 15L-16L, 16L-17L, 17L-18R, 18R-G1L, G1L-G3L, G3L-G2R, G2R-G4L, G4L-G5R, G5R-G5.5R, G5.5R-G6R, G6R-G7L, G7L-G8R, G8R-G9R, G9R-L1R, L1R-L2R, L2R-L3L, L3L-L4R, L4R-L5R, L5R-J1R, J3R-J4R, J4R-J5L, J5L-J6R, J6R-H1L, H1L-H2R, H2R-H3L, H3L-H4L, H4L-H5R, H5R-H6R, H6R-H7R, H7R-D1R, D1R-D2L, D2L-D3R, D3R-D4R, D4R-D5R, D5R-D6R, D6R-D7R, D9R-D10R, D10R-D11L, D11 L-D12L, D12L-D13L, D13L-A1L, A1L-A2L, A2L-A2.5L, A2.5L-A3L, A3L-A4L, A4L-ASR, A5R-A6L, A6L-A7L, A7L-A8R, A8R-A9L, A9L-A10L, A10L-A11R, A11R-A12L, A12L-A13L, A13L-A14L, A14L-A14.5L, A14.5L-A15L, A15L-A16L, A16L-A17L, A17L-A18R, A18R-A19L, A19L-A21L, A21 L-A20R, A20R-A22R, A22R-A23R, A23R-A24R, A28L-A29L and A29L-A30L, and furthermore 001 L-002L, 002L-003L, 005R-006R, 006L-007R, 007R-008L, 008L-009L, 017L-018L, 018L-019L, 019L-020L, 020L-021 L, 023L-024L, 024L-025L, 025L-026L, 028R-029L, 030L-031 L, 031 L-032L, 032L-033L, 035L-036L, 036L-037L, 037L-038L, 039L-040L, 043L-044L, 044L-045L, 046L-047R, 049L-050L, 050L-051 L, 051L-052R, 052R-053R, 053R-054R, 054R-055R, 055R-056L, 061 L-062L, 064L-065L, 065L-066L, 066L-067L, 077L-078R, 078R-079R, 080R-081R, 081R-082L, 082L-083R, 085R-086R, 086R-087R, 088R-089L, 089L-090R, 092R-093L, 094L-095R, 096R-097R, 097R-098R, 101R-102R, 103R-104R, 105L-106R, 107R-108L, 108L-109L, 109L-110L, 110L-111L, 113L-114L, 114L-115L, 115L-116R, 117L-118L, 118L-119R, 122R-123L, 123L-124L, 124L-125L, 125L-126L, 133R-134R, 134R-135R, 136L-137L, 137L-138L, 141L-142R, 143L-144R, 144R-145R, 145R-146R, 146R-147R, 147R-148R, 148R-149L, 152R-153L, 153L-154R, 154R-155R, 156R-157L, 157L-158R, 159R-160L, 160L-161R, 162R-163R, 163R-164R, 164R-165R, 165R-166R, 166R-167R, 167R-168R, 170R-171R, 173R-174R, 175R-176R, 176R-177R, 178R-179R, 179R-180R, 180R-181R, 183R-184R, 184R-185L, 185L-186R, 186R-187R, 187R-188R, 188R-189R, 189R-190R, 192R-193R where according to the old nomenclature, ORF 006L corresponds to Cl 0L, 019L corresponds to C6L, 020L to N1L, 021L to N2L, 023L to K2L, 028R to K7R, 029L to F1 L, 037L to F8L, 045L to F15L, 050L to E3L, 052R to E5R, 054R to E7R, 055R to E8R, 056L to E9L062L to I1 L, 064L to 14L, 065L to I5L, 081R to L2R, 082L to L3L, 086R to J2R, 087 to J3R, 088R to J4R, 089L to J5L, 092R to H2R, 095R to H5R, 107R to D10R, 108L to D11L, 122R to A11R, 123L to A12L, 125L to A14L, 126L to A15L, 135R to A24R, 136L to A25L, 137L to A26L, 141L to A30L, 148R to A37R, 149L to A38L, 152R to A40R, 153L to A41 L, 154R to A42R, 157L to A44L, 159R to A46R, 160L to A47L, 165R to A56R, 166R to A57R, 167R to B1R, 170R to B3R, 176R to B8R, 180R to B12R, 184R to B16R, 185L to B17L, and 187R to B19R, such a
composition wherein the attenuated vaccinia virus comprises deletion of one or more genes selected from the group consisting of a vaccinia virus A41 L gene, a vaccinia virus D13L gene, vaccinia virus B7R-B8R genes, a vaccinia virus A39R gene and a vaccinia virus C3L gene, such a
composition wherein the at least one nucleic acid sequence encoding a human coronavirus SARS-CoV-2 polypeptide is inserted into at least one deletion site of the one or more genes, such a
composition wherein a human coronavirus SARS-CoV-2 spike protein polypeptide, or an immunogenic part thereof, is inserted into a vaccinia virus A41 L gene deletion site, such a
composition wherein a human coronavirus SARS-CoV-2 membrane protein polypeptide or an immunogenic part thereof, and a nucleocapsid protein polypeptide or an immunogenic part thereof, is inserted into a vaccinia virus D13L gene deletion site, such a
composition wherein a human coronavirus SARS-CoV-2 envelope protein polypeptide, or an immunogenic part thereof, is inserted into vaccinia virus B7R-B8R gene deletion site, such a
composition wherein the at least one nucleic acid sequence encoding a human coronavirus SARS-CoV-2 polypeptide is inserted into an intergenic region (IGR) of the attenuated vaccinia virus genome, wherein the IGR is located between or is flanked by two adjacent ORFs of the vaccinia virus genome, such a
composition wherein the human coronavirus SARS-CoV-2 polypeptide is encoded by one or more expression cassettes having a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8, such a
composition wherein the human coronavirus SARS-CoV-2 polypeptide is encoded by an expression cassette having the nucleic acid sequence set forth in SEQ ID NO:1 and an expression cassette having the nucleic acid sequence set forth in SEQ ID NO:4, such a
composition wherein the human coronavirus SARS-CoV-2 polypeptide is encoded by an expression cassette having the nucleic acid sequence set forth in SEQ ID NO:2 and an expression cassette having the nucleic acid sequence set forth in SEQ ID NO:4, such a
composition wherein the human coronavirus SARS-CoV-2 polypeptide is encoded by an expression cassette having the nucleic acid sequence set forth in SEQ ID NO:1, such a
composition wherein the human coronavirus SARS-CoV-2 polypeptide is encoded by an expression cassette having the nucleic acid sequence set forth in SEQ ID NO:2, such a
composition wherein the human coronavirus SARS-CoV-2 polypeptide is encoded by an expression cassette having the nucleic acid sequence set forth in SEQ ID NO:5, such a
composition wherein the human coronavirus SARS-CoV-2 polypeptide is encoded by an expression cassette having the nucleic acid sequence set forth in SEQ ID NO:3, such a
composition wherein the human coronavirus SARS-CoV-2 polypeptide is encoded by an expression cassette having the nucleic acid sequence set forth in SEQ ID NO:4, such a
composition wherein the human coronavirus SARS-CoV-2 polypeptide is encoded by an expression cassette having the nucleic acid sequence set forth in SEQ ID NO:1 and an expression cassette having the nucleic acid sequence set forth in SEQ ID NO:4 and an expression cassette having the nucleic acid sequence set forth in SEQ ID NO:8, such a
composition wherein the human coronavirus SARS-CoV-2 polypeptide is encoded by an expression cassette having the nucleic acid sequence set forth in SEQ ID NO:2 and an expression cassette having the nucleic acid sequence set forth in SEQ ID NO:4 and an expression cassette having the nucleic acid sequence set forth in SEQ ID NO:8, such a
composition wherein the human coronavirus SARS-CoV-2 polypeptide is encoded by an expression cassette having the nucleic acid sequence set forth in SEQ ID NO:3 and an expression cassette having the nucleic acid sequence set forth in SEQ ID NO:4, such a
composition wherein the human coronavirus SARS-CoV-2 polypeptide is encoded by an expression cassette having the nucleic acid sequence set forth in SEQ ID NO:3 and an expression cassette having the nucleic acid sequence set forth in SEQ ID NO:4 and an expression cassette having the nucleic acid sequence set forth in SEQ ID NO:8, such a
composition wherein the human coronavirus SARS-CoV-2 polypeptide is encoded by an expression cassette having the nucleic acid sequence set forth in SEQ ID NO:6, such a
composition wherein the human coronavirus SARS-CoV-2 polypeptide is encoded by an expression cassette having the nucleic acid sequence set forth in SEQ ID NO:7, such a
composition comprising a pharmaceutically acceptable carrier or diluent, such a
composition for raising an immune response in animal which decreases the risk of a coronavirus disease, the composition comprising a genetically engineered attenuated vaccinia virus, wherein the vaccinia virus genome comprises a nucleic acid sequence encoding a spike protein polypeptide or an immunogenic part thereof, of human coronavirus SARS-CoV-2, and wherein the attenuated vaccinia virus comprises a deletion of at least one gene which encodes an endogenous essential assembly or maturation protein, admixed with a second genetically engineered attenuated vaccinia virus, wherein the second vaccinia virus genome comprises a nucleic acid sequence encoding a membrane protein polypeptide and nucleocapsid protein polypeptide or immunogenic part or parts thereof, of human coronavirus SARS-CoV-2, and wherein the second attenuated vaccinia virus comprises a deletion of at least one gene which encodes an endogenous essential assembly or maturation protein, such a
genetically engineered attenuated vaccinia virus vector, wherein the vaccinia virus genome comprises a nucleic acid sequence encoding a spike protein polypeptide, a membrane protein polypeptide and a nucleocapsid protein polypeptide, and/or an envelope protein polypeptide of human coronavirus SARS-CoV-2, wherein the attenuated vaccinia virus vector expresses the aforementioned polypeptides which assemble into virus-like-particles.

A method for preventing or decreasing the risk of SARS-CoV-2 infection comprising administering a composition comprising an attenuated vaccinia virus, wherein the vaccinia virus genome comprises a nucleic acid sequence encoding a human coronavirus SARS-CoV-2 spike polypeptide or immunogenic part thereof, and a membrane protein polypeptide or immunogenic part thereof, and a nucleocapsid protein polypeptide or immunogenic part thereof, and optionally, an envelope protein polypeptide or immunogenic part thereof, to an animal, including a human, in an amount effective to elicit an immune response directed against SARS-CoV-2, such a
method wherein the immune response directed against SARS-CoV-2 antigens provides antibodies which are cross-reactive against coronaviruses with genetic similarity to SARS-CoV-2.

The use of a composition as any above in the preparation of a medicament for use in inducing a protective immune response in a subject against a coronavirus infection.

### BRIEF DESCRIPTION OF FIGURES

FIG. 1: Poxvirus expression cassettes were synthesised (A, B, F and G) or constructed by PCR (C, D and E) for SARS-CoV-2 S, M, N and E.
A. Expression cassette comprising SARS-CoV-2 spike protein under transcriptional control of synthetic early/late promoter (prPs).
B. Expression cassette comprising SARS-CoV-2 spike protein under transcriptional control of native early/late promoter (pr7.5).
C. Expression cassette comprising SARS-CoV-2 S1 subunit of the spike protein under transcriptional control of synthetic early/late promoter (prPs).
D. Expression cassette comprising SARS-CoV-2 membrane protein under transcriptional control of fowlpox early/late promoter (prE/L).
E. Expression cassette comprising SARS-CoV-2 nucleocapsid protein under transcriptional control of synthetic early/late promoter (prPs).
F. Expression cassette comprising SARS-CoV-2 membrane protein under transcriptional control of fowlpox early/late promoter (prE/L) and SARS-CoV-2 nucleocapsid protein under transcriptional control of synthetic early/late promoter (prPs).
G. Expression cassette comprising SARS-CoV-2 envelope protein under transcriptional control of synthetic early/late promoter (prPs).

FIG. 2: Homologous recombination sites indicated by F1 and F2 recombination arms relative to the vaccinia virus Copenhagen strain (VACV-COP) genome.
A. A39R region of VACV-COP with F1-A39R and F2-A39R homologous recombination arms that flank the VACV-COP region deleted in SCV-SMX06 and between which SARS-CoV-2 antigens may be inserted.
B. A41L region of VACV-COP with F1-A41L and F2-A42L homologous recombination arms that flank the VACV-COP region deleted in SCV-SMX06 and between which SARS-CoV-2 antigens may be inserted.
C. B7/B8R region of VACV-COP with F1-B7B8R and F2-B7B8R homologous recombination arms that flank the VACV-COP region deleted in SCV-SMX06 and between which SARS-CoV-2 antigens may be inserted.
D. C3L region of VACV-COP with F1-C3L and F2-C3L homologous recombination arms that flank the VACV-COP region deleted in SCV-SMX06 and between which SARS-CoV-2 antigens may be inserted.
E. D13L region of VACV-COP with F1-D13L and F2-D13L homologous recombination arms that flank the VACV-COP region deleted in SCV-SMX06 and between which SARS-CoV-2 antigens may be inserted.
F. J2R-J3R intergenic region of VACV-COP with F1-J2/J3R and F2-J2/J3R homologous recombination arms that flank the VACV-COP region between which SARS-CoV-2 antigens may be inserted.

FIG. 3: Detailed map and elements of homologous recombination (HR) cassettes for SARS-CoV-2 transgenes.
A. HR cassette for A41 L replacement comprising prPs SARS-CoV-2 spike expression cassette.
B. HR cassette for A41L replacement comprising pr7.5 SARS-CoV-2 spike expression cassette.
C. HR cassette for A41 L replacement comprising prPs SARS-CoV-2 S1 subunit of the spike expression cassette.
D. HR cassette for D13L replacement comprising prE/L SARS-CoV-2 membrane and prPs SARS-CoV-2 nucleocapsid expression cassette.
E. HR cassette for insertion into intergenic site J2/J3R comprising prE/L SARS-CoV-2 membrane and prPs SARS-CoV-2 nucleocapsid expression cassette.
F. HR cassette for C3L replacement comprising prE/L SARS-CoV-2 membrane expression cassette.
G. HR cassette for D13L replacement comprising prPs SARS-CoV-2 nucleocapsid expression cassette.
H. HR cassette for B7/B8R replacement comprising prPs SARS-CoV-2 envelope expression cassette.

FIG. 4: Schematic of vaccine construction process.

FIG. 5: SARS-CoV-2 antigen insertion regions within SCV-COVID19 vaccines.
A. SARS-CoV-2 spike transgene under transcriptional control of synthetic early/late promoter substituted into the A41 L ORF.
B. SARS-CoV-2 spike transgene under transcriptional control of native early/late promoter substituted into the A41L ORF.
C. SARS-CoV-2 S1 subunit of the spike transgene substituted into the A41 L ORF.
D. SARS-CoV-2 membrane and nucleocapsid transgene replaced the D13L ORF.
E. SARS-CoV-2 membrane and nucleocapsid transgene inserted in the intergenic region between the J2R and J3R ORFs.
F. SARS-CoV-2 envelope transgene replaced the B7/B8R ORF.
G. SARS-CoV-2 membrane transgene replaced the C3L ORF.
H. SARS-CoV-2 nucleocapsid transgene replaced the D13L ORF.

FIG. 6: Single vaccination with SCV-COVID19D generates neutralizing SARS-CoV-2 antibodies and a Th1-biased antibody profile in outbred and inbred mice.
A. Levels of virus-specific neutralizing antibodies in outbred ARC(s) and inbred C57BL/6 strains on day 21 following SCV-COVID19D single shot vaccination.
B. Levels of S1-specific antibodies in outbred ARC(s) and inbred C57BL/6 strains on day 21 following SCV-COVID19D single shot vaccination.
C. Levels of IgG2c (Th1) and IgG1 (Th2) antibodies in outbred ARC(s) and inbred C57BL/6 strains on day 21 following SCV-COVID19D single shot vaccination.

FIG. 7: Single vaccination with SCV-COVID19D generates spike-specific CD8 T cell responses.
A. Level of spike-specific IFNγ⁺ CD8 T cells by intracellular cytokine staining (ICS) following SCV-COVID19D single shot vaccination.
B. Level of spike-specific IFNγ⁺ CD8 T cells by ELISpot following SCV-COVID19D single shot vaccination.

FIG. 8: SCV-COVD19C elicits better spike-specific antibody than SCV-COVID19D
A. Detection by western blot of spike antigen expression in cells infected with SCV-COVID19 vaccine.
B. Levels of S1-specific antibodies on day 21 following SCV-COVID19C, SCV-COVID19D, and SCV-COVID19F single shot vaccination.

FIG. 9: Single vaccination with SCV-COVID19C induces antibody responses in inbred and outbred mice.
A. Levels of S1-specific antibodies in outbred ARC(s) and inbred C57BL/6 strains on day 14 following SCV-COVID19C single shot vaccination.
B. Titres of S1-specific antibodies in outbred ARC(s) and inbred C57BL/6 strains on day 14 following SCV-COVID19C single shot vaccination.
C. Levels of neutralizing antibodies in outbred ARC(s) and inbred C57BL/6 strains on day 14 following SCV-COVID19C single shot vaccination.

FIG. 10: Gating strategy for identification of triple-cytokine-producing CD8 T cells FIG. 11: Single vaccination with SCV-COVID19C induces robust spike-specific T cell response.
A. Representative flow cytometric plots showing detection of spike-specific IFNγ⁺ CD8 T cells following SCV-COVID19C single shot vaccination.
B. Graphical summary of the total number of single, double, and triple-cytokine-producing IFNγ⁺ CD8 T cells that are spike pool 1 (S1)-specific (left), spike pool 2 (S2)-specific (middle), and epitope-specific (right) following SCV-COVID19C single shot vaccination.
C. Representative flow cytometric plots and graphical summary of granzyme-B-producing CD8 T cells following SCV-COVID19C single shot vaccination D. Total number of 51- and S2-specific triple-cytokine-producing CD4 T cells following SCV-COVID19C single shot vaccination.

FIG. 12: Pre-existing immunity does not affect the quantity and quality of spike-specific antibody responses following administration of a single-dose of SCV-COVID19C vaccine.
A. Levels of S1-specific antibodies for mice with and without pre-existing immunity on days 28, 44, and 80 following SCV-COVID19C single shot vaccination
B. Levels of neutralizing antibodies for mice with and without pre-existing immunity on days 28, 44, and 80 following SCV-COVID19C single shot vaccination.

FIG. 13: Pre-existing immunity does not affect quantity and quality of spike-specific antibody responses after prime-boost vaccination.
A. Levels of S1-specific antibodies for mice with and without pre-existing immunity on days 28 (pre-boost) and days 14 and 50 post-booster dose in SCV-COVID19C homologous prime-boost vaccination.
B. Levels of neutralizing antibodies for mice with and without pre-existing immunity on days 28 (pre-boost) and days 14 and 50 post-booster dose in SCV-COVID19C homologous prime-boost vaccination.

FIG. 14: Single vaccination with SCV-COVID19C induces antigen-specific antibody response in aging mice.
A. Levels of S1-specific antibodies in young and aging mice on days 14 and 21 following SCV-COVID19C single shot vaccination.
B. Levels of neutralizing antibodies in young and aging mice on days 14 and 21 following SCV-COVID19C single shot vaccination.

FIG. 15: Homologous prime-boost leads to a significant boosting of antibody responses that is maintained for up to 3 months post-vaccination.
A. Levels of S1-specific antibodies in young and aging mice on day 21 following SCV-COVID19C single shot vaccination and on day 21 post-boost following SCV-COVID19C homologous prime-boost vaccination.
B. Levels of neutralizing antibodies in young and aging mice on day 21 following SCV-COVID19C single shot vaccination and on day 21 post-boost following SCV-COVID19C homologous prime-boost vaccination.
C. Levels of neutralizing antibodies in young and aging mice on weeks 3, 9, and 12 post-boost following SCV-COVID19C homologous prime-boost vaccination.

FIG. 16: Gating strategy for flow cytometric identification of T cell memory cell types using cell surface markers.

FIG. 17: Homologous prime-boost of SCV-COVID19C induces long term T cell response.
A. Total number of short-lived effector (T_{SLE}), effector memory (T_{EM}), and central memory (T_{CM}) CD8 T cells 3 months following SCV-COVID19C single shot vaccination and 3 months post-boost following SCV-COVID19C homologous prime-boost vaccination in young and aging mice.
B. Levels of spike-specific IFNγ⁺ CD8 T cells 3 months following SCV-COVID19C single shot vaccination and 3 months post-boost following SCV-COVID19C homologous prime-boost vaccination in young and aging mice.
C. Levels of S1-, RBD-, and S2-specific IFNγ⁺ spot-forming units detected by ELISpot 3 months following SCV-COVID19C single shot vaccination and 3 months post-boost following SCV-COVID19C homologous prime-boost vaccination in young and aging mice.
D. Percentages of S1-, RBD-, and S2-specific IFNγ⁺ CD8 T cells detected by ICS 3 months following SCV-COVID19C single shot vaccination and 3 months post-boost following SCV-COVID19C homologous prime-boost vaccination in young and aging mice.
E. Total number of S1-, RBD-, and S2-specific triple-cytokine-producing CD8 T cells detected by ICS 3 months following SCV-COVID19C single shot vaccination and 3 months post-boost following SCV-COVID19C homologous prime-boost vaccination in young and aging mice.

FIG. 18: Homologous prime-boost of SCV-COVID19C potentially can potentially cross-react with SARS-CoV based on CD8 T cell epitopes in the spike RBD.
A. Levels of epitope-specific IFNγ⁺ CD8 T cells by ELISpot on day 7 following SCV-COVID19C single shot vaccination.
B. Representative flow cytometric panels showing epitope-specific IFNγ⁺ CD8 T cells by ICS on day 7 following SCV-COVID19C single shot vaccination.

FIG. 19: Single vaccination with SCV-COVID19A generates spike- and membrane-specific CD8 T cell responses.
A. Levels of S1-, RBD-, and S2-specific IFNγ⁺ CD8 T cells on day 21 following SCV-COVID19A single shot vaccination.
B. Levels of membrane-specific IFNγ⁺ CD8 T cells on day 21 following SCV-COVID19A single shot vaccination.
C. Levels of nucleocapsid-specific IFNγ⁺ CD8 T cells on day 21 following SCV-COVID19A single shot vaccination.

FIG. 20: Single vaccination with equal proportions of SCV-COVID19C and SCV-COVID19G induces spike-specific antibody responses and CD8+ T cell responses directed towards the spike and membrane proteins.
A. Levels of S1-specific antibodies on day 21 following a single shot vaccination with a mixed vaccine comprising of SCV-COVID19C and SCV-COVID19G.
B. Titres of S1-specific antibodies on day 21 following a single shot vaccination with a mixed vaccine comprising of SCV-COVID19C and SCV-COVID19G.
C. Level of spike-specific IFNγ⁺ producing T cell response by intracellular cytokine staining (ICS) following a single shot vaccination with a mixed vaccine comprising of SCV-COVID19C and SCV-COVID19G.
D. Level of spike and membrane-specific IFNγ⁺ producing T cell response by ELISpot following a single shot vaccination with a mixed vaccine comprising of SCV-COVID19C and SCV-COVID19G.

FIG. 21: Single vaccination with SCV-COVD19C generates epitope-specific cytotoxic T lymphocyte (CTL) activity.
A. CTL response against target cells pulsed with peptide YNYLYRLF (SEQ ID NO:9) at day 7 post-vaccination.
B. CTL response against target cells pulsed with peptide VNFNFNGL (SEQ ID NO:11) at day 7 post-vaccination.
C. CTL response against non-pulsed target cells.

### DETAILED DESCRIPTION

The subject invention is not limited to particular procedures or agents, specific formulations of agents and various medical methodologies, as such may vary. The terminology used herein is for the purpose of describing particular embodiments only and is not intended to be limiting. Unless defined otherwise, all technical and scientific terms used herein have the same meanings as commonly understood by one of ordinary skill in the art to which this invention belongs.

Any materials and methods similar or equivalent to those described herein can be used to practice or test the present invention. Practitioners are particularly directed to: Sambrook et al., (1989) Molecular Cloning: A Laboratory Manual, 2^{nd} ed., Cold Spring Harbor Press, Plainsview, N.Y.: Ausubel et al. (1999) Current Protocols in Molecular Biology (Supplement 47) John Wiley & Sons, New York; Murphy et al (1995) Virus Taxonomy Springer Verlag: 79-87, Mahy Brian W J and Kangro O Hillar (Eds): Virology Methods Manual 1996, Academic Press; and Davison A J and Eliott R M (Eds): Molecular Virology, A Practical Approach 1993, IRL Press at Oxford University Press; Perkus et al., Virology (1990) 179(1): 276-86 or definitions and terms of the art and other methods known to the person skilled in the art.

Although any methods and materials similar or equivalent to those described herein can be used in the practice or testing of the present invention, preferred methods and materials are described. For the purposes of the present invention, the following terms are defined below.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. In the context of attenuated orthopox vectors, the subject vectors are modified for attenuation by comprising deletion of an

maturation or assembly gene however, further modification such as to vector an antigen or other protein is encompassed.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity of action of the listed elements.

As used herein the singular forms “a”, “an” and “the” include plural aspects unless the context clearly dictates otherwise. Thus, for example, reference to “a cell” includes a single cell, as well as two or more cells; reference to “an organism” includes one organism, as well as two or more organisms; and so forth. In some embodiments, “an” means “one or more than one”.

As used herein, “and/or” refers to and encompasses any and all possible combinations of one or more of the associated listed items, as well as the lack of combinations when interpreted in the alternative (or).

“Attenuation” or “attenuated” as used herein means a reduction of viral vector virulence. Virulence is defined as the ability of a virus to cause disease in a particular host. A poxviral vector that is unable to produce infectious viruses may initially infect cells but is unable substantially to replicate itself fully or propagate within the host or cause a condition. This is desirable as the vector delivers its protein or nucleic acid to the host cell cytoplasm but does not harm the subject.

By “control element” or “control sequence” is meant nucleic acid sequences (e.g., DNA) necessary for expression of an operably linked coding sequence in a particular poxvirus, vector, plasmid or cell. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Where sequences are provided, corresponding sequences are encompassed. By “corresponds to” “corresponding” or “corresponding to” is meant a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 m 84, 85, 86, 87, 88, 89, 90, 91, 92, 93, 94, 95, 96, 96, 98, 99% or even up to 100% sequence identity to all or a portion of the reference nucleic acid sequence) or an amino acid sequence that displays substantial sequence similarity or identity to a reference amino acid sequence (e.g., at least 50, 51, 52, 53, 54, 55, 56, 57, 58, 59, 60, 61, 62, 63, 64, 65, 66, 67, 68, 69, 70, 71, 72, 73, 74, 76, 77, 78, 79, 80, 81, 82, 83 m 84, 85, 86, 87, 88, 89, 90, 91, 92, 93, 94, 95, 96, 96, 98, 99% or even up to 100% sequence similarity or identity to all or a portion of the reference amino acid sequence).

By “effective amount”, in the context of treating or preventing a condition or for modulating an immune response to a target antigen or organism is meant the administration of an amount of an agent (e.g., an attenuated orthopox vector as described herein) or composition comprising same to an individual in need of such treatment or prophylaxis, either in a single dose or as part of a series, that is effective for the prevention of incurring a symptom, holding in check such symptoms, and/or treating existing symptoms, of that condition or for modulating the immune response to the target antigen or organism. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the terms “encode,” “encoding” and the like refer to the capacity of a nucleic acid to provide for another nucleic acid or a polypeptide. For example, a nucleic acid sequence is said to “encode” a polypeptide or if it can be transcribed and/or translated to produce the polypeptide or if it can be processed into a form that can be transcribe and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The term “endogenous” refers to a gene or nucleic acid sequence or segment that is normally found in a host organism.

The terms “expressible,” “expressed,” and variations thereof refer to the ability of a cell to transcribe a nucleotide sequence to RNA and optionally translate the mRNA to synthesize a peptide or polypeptide that provides a biological or biochemical function.

As used herein, the term “gene” includes a nucleic acid molecule capable of being used to produce mRNA optionally with the addition of elements to assist in this process. Genes may or may not be capable of being used to produce a functional protein. Genes can include both coding and non-coding regions (e.g., introns, regulatory elements, promoters, enhancers, termination sequences and 5′ and 3′ untranslated regions).

The terms “heterologous nucleic acid sequence,” “heterologous nucleotide sequence,” “heterologous polynucleotide,” “foreign polynucleotide,” “exogenous polynucleotide” and the like are used interchangeably to refer to any nucleic acid (e.g., a nucleotide sequence comprising an IRES) which is introduced into the genome of an organism by experimental manipulations and may include gene sequences found in that organism so long as the introduced gene contains some modification (e.g., a point mutation, deletion, substitution or addition of at least one nucleoide, the presence of a endonuclease cleavage site, the presence of a IoxP site, etc.) relative to the viral genomic sequence before the modification.

The terms “heterologous polypeptide,” “foreign polypeptide” and “exogenous polypeptide” are used interchangeably to refer to any peptide or polypeptide which is encoded by a “heterologous nucleic acid sequence,” “heterologous nucleotide sequence,” “heterologous polynucleotide,” “foreign polynucleotide” and “exogenous polynucleotide,” as defined above.

In an embodiment, the heterologous DNA sequence comprises at least one coding sequence. The coding sequence is operatively linked to a transcription control element.

In an embodiment, the heterologous DNA sequence can also comprise two or more coding sequences linked to one or several transcription control elements. Preferably, the coding sequence encodes one or more proteins, polypeptides, peptides, foreign antigens or antigenic epitopes, especially those of therapeutically interesting genes. Therapeutically interesting genes may be derived from or homologous to genes of pathogen or infectious microorganisms which are disease causing. Therapeutically interesting genes are presented to the immune system of an organism in order to affect, preferably induce a specific immune response and, thereby, vaccinate or prophylactically protect the organism against an infection.

In an embodiment, the heterologous DNA sequence is derived from SARS-CoV-2 and encodes the spike protein, and/or membrane protein, and/or nucleocapsid protein, and/or envelope protein or part or parts thereof of any.

The term “protective immune response” means an immune response which prevents or decreases the risk of SARS-CoV-2 infection or decreases the risk of severity of coronavirus disease.

The immune response directed against SARS-CoV-2 antigens may provide antibodies which are cross-reactive against coronaviruses with genetic similarity to SARS-CoV-2.

The term “neutralizing antibody response” means an immune response in which antibodies are elicited which can neutralize viral infectivity. Generating neutralizing antibodies through vaccination can be both sufficient and necessary for protection against viral infections. The presence of neutralizing antibodies is the best correlate of protection from viral infection after vaccination. Likewise, they are markers of immunity.

It will be understood that inducing an immune response as contemplated herein includes eliciting or stimulating an immune response and/or enhancing a previously existing immune response.

An immune response in animal which prevents or decreases the risk of SARS-CoV-2 infection and/or severity of a coronavirus disease may be mediated through prevention or reduction of SARS-CoV-2 transmission.

The pox virus vector of the present invention is preferably propagated in a mammalian cell. Details of the mammalian cells which can be used in the present invention are provided in PCT/AU2014/050330, the disclosure of which is incorporated herein by cross reference.

In some embodiments, the mammalian cell is a human cell, a primate cell, a hamster cell or a rabbit cell.

Cells may be unicellular or can be grown in tissue culture as liquid cultures, monolayers or the like. Host cells may also be derived directly or indirectly from tissues or may exist within an organism including animals. Cells may be established cell lines, including cell lines which have been modified to express ubiquitinated T cell antigens.

In some embodiments, homologous recombination and/or viral propagation is carried out in BC19A-12 cell line, an SCV cell substrate derived from GMP-CHO-S cell line expressing D13L protein and a cowpox host-range protein (CP77). The SCV vaccine platform incorporates a targeted deletion of the D13L gene in the viral genome to prevent viral assembly, thereby rendering SCV unable to generate infectious progeny in normally permissive cell lines; however, amplification of the SCV genome is retained. CHO cells were engineered to constitutively express D13 and CP77, thereby permitting viral propagation. For a complete description of the method of viral propagation using the BC19A-12 cell line, refer to Eldi et al. 2017 incorporated herein in its entirety.

In some embodiments, homologous recombination and/or viral propagation is carried out in SD07-1 cell line, a monoclonal suspension CHO cell line that constitutively expresses the vaccinia virus D13 protein and can grow in protein or serum free medium.

The term “operably connected” or “operably linked” as used herein refers to a juxtaposition wherein the components so described are in a relationship permitting them to function in their intended manner. For example, a transcriptional control sequence “operably linked” to a coding sequence refers to positioning and/or orientation of the transcriptional control sequence relative to the coding sequence to permit expression of the coding sequence under conditions compatible with the transcriptional control sequence. In another example, an RES operably connected to an orthopox virus coding sequence refers to positioning and/or orientation of the IRES relative to the orthoxpox virus coding sequence to permit cap-independent translation of the orthopox virus coding sequence.

As used here the terms “open reading frame” and “ORF” are used interchangeably herein to refer to the amino acid sequence encoded between translation initiation and termination codons of a coding sequence. The terms “initiation codon” (e.g., ATG) and “termination codon” (e.g., TGA, TAA, TAG) refer to a unit of three adjacent nucleotides (‘codon’) in a coding sequence that specifies initiation and chain termination, respectively, of protein synthesis (mRNA translation).

The terms “polynucleotide,” “polynucleotide sequence,” “nucleotide sequence,” “nucleic acid” or “nucleic acid sequence” as used herein designate mRNA, RNA, cRNA, cDNA, or DNA. The term typically refers to polymeric form of nucleotides of at least 10 bases in length, either ribonucleotides or deoxynucleotides or a modified form of either type of nucleotide. The term includes single and double stranded forms of RNA or DNA.

“Polypeptide,” “peptide,” “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

As used herein the term “recombinant” as applied to “nucleic acid molecules,” “polynucleotides” and the like is understood to mean artificial nucleic acid structures (i.e., non-replicating cDNA or RNA; or replicons, self-replicating cDNA or RNA) which can be transcribed and/or translated in host cells or cell-free systems described herein. Recombinant nucleic acid molecules or polynucleotides may be inserted into a vector. Non-viral vectors such as plasmid expression vectors or viral vectors may be used. The kind of vectors and the technique of insertion of the nucleic acid construct according to this invention is known to the artisan. A nucleic acid molecule or polynucleotide according to the invention does not occur in nature in the arrangement described by the present invention. In other words, an heterologous nucleotide sequence is not naturally combined with elements of a parent virus genome (e.g., promoter, ORF, polyadenylation signal, ribozyme).

As used herein, the term “recombinant virus” will be understood to be a reference to a “parent virus” comprising at least one heterologous nucleic acid sequence.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hiachi Software engineering Co., Ltd., South San Francisco, California, USA) using standard defaults as used in the reference manual accompanying the software.

The terms “signal sequence” or “signal peptide” refers to a short (approximately 3 to about 60 amino acids long) peptide that directs co- or post-translational transport of a protein from the cytosol to certain organelles such as the nucleus, mitochondrial matrix, and endoplasmic reticulum, for example. For proteins having an ER targeting signal peptide, the signal peptides are typically cleaved from the precursor form by signal peptidase after the proteins are transported to the ER, and the resulting proteins move along the secretory pathway to their intracellular (e.g., the Golgi apparatus, cell membrane or cell wall) or extracellular locations. “ER targeting signal peptides,” as used herein include amino-terminal hydrophobic sequences which are usually enzymatically removed following the insertion of part or all of the protein through the ER membrane into the lumen of the ER. Thus, it is known in the art that a signal precursor form of a sequence can be present as part of a precursor form of a protein, but will generally be absent from the mature form of the protein.

“Similarity” refers to the percentage number of amino acids that are identical or constitutively conserved substitutions as defined in Table A below. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

TABLE A
Exemplary Conservative Amino Acid Substitutions

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The terms “subject,” “patient,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (eg., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri), ferrets (species from the genus Mustela) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of treatment or prophylaxis of a condition. However, it will be understood that the aforementioned terms do not imply that symptoms are present.

The term “transgene” is used herein to describe a genetic material that has been or is about to be artificially introduced into a genome of a host organism and that is transmitted to the progeny of that host. In some embodiments, it confers a desired property to a mammalian cell or an orthopox vector into which it is introduced, or otherwise leads to a desired therapeutic or diagnostic outcome.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “wild-type,” “natural,” “native” and the like with respect to an organism, polypeptide, or nucleic acid sequence, that the organism polypeptide, or nucleic acid sequence is naturally occurring or available in at least one naturally occurring organisms which is not changed, mutated, or otherwise manipulated by man.

The term “viral infection” means an infection by a viral pathogen in a biological sample from the subject.

The term “virus-like particles” or “VLP” refers to a structure which resembles the native virus antigenically and morphologically.

Variants include nucleic acid molecules sufficiently similar to a referenced molecule or their complementary forms over all or part thereof such that selective hybridization may be achieved under conditions of medium or high stringency, or which have about 60% to 90% or 90% to 98% sequence identity to the nucleotide sequences defining a referenced poxvirus host range factor over a comparison window comprising at least about 15 nucleotides. Preferably the hybridization region is about 12 to about 18 nucleobases or greater in length. Preferably, the percent identity between a particular nucleotide sequence and the reference sequence is at least about 80%, or 85%, or more preferably about 90% similar or greater, such as about 95%, 96%, 97%, 98%, 99% or greater. Percent identities between 80% and 100% are encompassed. The length of the nucleotide sequence is dependent upon its proposed function. Homologs are encompassed. The term “homolog” “homologous genes” or “homologs” refers broadly to functionally and structurally related molecules including those from other species. Homologs and orthologs are examples of variants.

Nucleic acid sequence identity can be determined in the following manner. The subject nucleic acid sequence is used to search a nucleic acid sequence database, such as the GenBank database (accessible at website www.ncbi.nln.nih.gov/blast/), using the program BLASTM version 2.1 (based on Altschul et al. (1997) Nucleic Acids Research 25:3389-3402). The program is used in the ungapped mode. Default filtering is used to remove sequence homologies due to regions of low complexity. The default parameters of BLASTM are used.

Amino acid sequence identity can be determined in the following manner. The subject polypeptide sequence is used to search a polypeptide sequence database, such as the GenBank database (accessible at website www.ncbi.nln.nih.gov/blast/), using the BLASTP program. The program is used in the ungapped mode. Default filtering is used to remove sequence homologies due to regions of low complexity. The default parameters of BLASTP are utilized. Filtering for sequences of low complexity may use the SEG program.

Preferred sequences will hybridize under stringent conditions to a reference sequence or its complement. The term “hybridize under stringent conditions”, and grammatical equivalents thereof, refers to the ability of a nucleic acid molecule to hybridize to a target nucleic acid molecule (such as a target nucleic acid molecule immobilized on a DNA or RNA blot, such as a Southern blot or Northern blot) under defined conditions of temperature and salt concentration. With respect to nucleic acid molecules greater than about 100 bases in length, typical stringent hybridization conditions are no more than 25° C. to 30° C. (for example, 10° C.) below the melting temperature (Tm) of the native duplex (see generally, Sambrook et al., (supra); Ausubel et al., (1999)). Tm for nucleic acid molecules greater than about 100 bases can be calculated by the formula Tm=81.5+0.41% (G+C−log (Na⁺)). With respect to nucleic acid molecules having a length less than 100 bases, exemplary stringent hybridization conditions are 5° C. to 10° C. below Tm.

The term “deletion” in the present context refers to removal of all or part of the coding region of the target gene. The term also encompasses any form of mutation or transformation which ablates gene expression of the target gene or ablates or substantially downregulates the level or activity of the encoded protein.

Reference to “gene” includes DNA corresponding to the exons or the open reading frame of a gene. Reference herein to a “gene” is also taken to include: a classical genomic gene consisting of transcriptional and/or translational regulatory sequences and/or a coding region and/or non-translated sequences (i.e. introns, 5′- and 3′-untranslated sequences); or mRNA or cDNA corresponding to the coding regions (i.e. exons) and 5′- and 3′-untranslated sequences of the gene.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promotor, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

Chimeric constructs suitable for effecting the present modified mammalian cells comprise a nucleic acid sequence encoding an orthopox host range factor, which is operably linked to a regulatory sequence. The regulatory sequence suitably comprises transcriptional and/or translational control sequences, which will be compatible for expression in the cell. Typically, the transcriptional and translational regulatory control sequences include, but are not limited to, a promoter sequence, a 5′ non-coding region, a cis-regulatory region such as functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′non-translated region. Constitutive or inducible promoters as known in the art are contemplated. The promoters may be either naturally occurring promoters, or hybrid promoters that combine elements or more than one promoter.

Promoter sequences contemplated may be native to mammalian cells or may be derived from an alternative source, where the region is functional in the chosen organism. The choice of promoter will differ depending on the intended host cell. For example, promoters which could be used for expression in mammalian cells include the metallothionein promoter, which can be induced in response to heavy metals such as cadmium, the β-actin promoter as well as viral promoters such as the SV40 large T antigen promoter, human cytomegalovirus (CMV) immediate early (IE) promoter, Rous sarcoma virus LTR promoter, the mouse mammary tumour virus LTR promoter, the adenovirus major late promoter (Ad MLP), the herpes simplex virus promoter, and a HPV promoter, particularly the HPV upstream regulatory region (URR), among other. All these promoters are well described and readily available in the art.

There are a number of poxviral promoter types which are distinguished by the time periods within the viral replication cycle in which they are active. Whereas early promoters can also be active late in infection, activity of late promoters is confined to the late phase. A third class of promoters named intermediate promoters is active at the transition of early to late phase and is dependent on viral DNA replication. Promoters which are active in both the early and late phases of the poxviral replication cycle are usually employed to direct the expression of neoantigens in poxvirus vectors. A compact, synthetic promoter (prPs) has been used widely to direct strong early as well as late gene expression. The pr7.5 promoter is another example of a native early-late promoter used for recombinant gene expression by vaccinia virus vectors.

As used herein, the terms “early/late promoter” refer to promoters that are active in virus infected cells pre- and post-viral DNA replication has occurred. Particularly preferred are poxvirus early/late promoter comprising synthetic vaccinia early/late promoter (Ps), native vaccinia early/late promoter (p7.5), and fowlpox early/late promoter (pE/L). Promoters as used herein are vaccinia virus promoters unless specified otherwise.

Enhancer elements may also be used herein to increase expression levels of the mammalian constructs. Examples include the SV40 early gene enhancer, as described for example in Dijkema et al. (1985) EMBO J. 4:761, the enhancer/promoter derived from the long terminal repeat (LTR) of the Rous Sarcoma Virus, as described for example in Gorman et al., (1982) Proc. Natl. Acad. Sci. USA 79:6777 and elements derived from human CMV, as described for example in Boshart et al. (1985) Cell 41:521, such as elements included in the CMV intron A sequence.

The chimeric construct may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nts and may contain transcriptional and translational termination sequences in addition to a polyadenylation signal and any other regulatory signals capable of effecting mRNA processing or gene expression.

In some embodiments, the chimeric construct further contains a selectable marker gene to permit selection of cells containing the construct. Selection genes are well known in the art and will be compatible for expression in the cell of interest.

In one embodiment, expression of the poxvirus structural or assembly gene is under the control of a promoter. In one non-liming embodiment the promoter is a cellular constitutive promoter, such as human EF1 alpha (human elongation factor 1 alpha gene promoter), DHFR (dihydrofolate reductase gene promoter) or PGK (phosphoglycerate kinase gene promoter) that direct expression of a sufficient level of CP77 to sustain viral propagation in the absence of significant toxic effects on the host cell. Promoters may also be inducible, such as the cellular inducible promoter, MTH (from a metallothionein gene) viral promoters are also employed in mammalian cells, such as CMV, RSV, SV-40, and MoU3.

The present invention provides a composition for a prophylactic vaccine against a novel coronavirus named Severe Acute Respiratory Syndrome Coronavirus-2 (SARS-CoV-2), the causative agent for the disease called Coronavirus disease 19 (COVID-19). COVID-19 was declared a pandemic by the World Health Organization (WHO) and has been impacting a large number of people worldwide. SARS-CoV-2 is a positive-sense single-stranded RNA virus. The SARS-CoV-2 genome is about 29,700 nucleotides long and has a 79.5% sequence similarity with SARS-CoV; it has a 5′ end long ORF1ab polyprotein that encodes 15 or 16 non-structural proteins while the 3′ end genome encodes the four main structural proteins (spike, nucleocapsid, membrane, and envelope). SARS-CoV-2 binds to angiotensin receptor conversion enzyme 2 (ACE2) expressed on host cells for viral entry and eventual pathogenesis. SARS-CoV-2 virus primarily affects the respiratory system with symptoms including fever, dry cough, dyspnea, headache, dizziness, generalized weakness, vomiting, and diarrhea. Current medical management is largely supportive with no targeted therapy available.

The inherent tendency of RNA viruses, including SARS-CoV-2, to change through mutation has been documented globally with new variants occurring over time. While most emerging mutations will not have a significant impact on the spread, mutations that confer selective advantage to the virus are generally retained as reflected in the increased prevalence of the variant in the population. Among the numerous SARS-CoV-2 variants recorded, a select few are of public health concern due to their increased transmissibility, capacity to inflict a more severe illness, and/or increased ability to elude the immune response that develops following infection or vaccination. In 2021, three specific viral lineages reflecting variants of concern (VOC) have emerged: B.1.1.7, B.1.351, and B.1.1.28.1. The mutation referred to as D614G is shared by the three variants of concern. It imparts to the virus increased transmissibility compared to other predominant viruses and other SARS-CoV-2 strains without this mutation.

As used herein, the variants of concern refer to the B.1.1.17, B.1.351, and B.1.1.28.1 specifically the P.1 lineage. The B.1.1.7 variant was initially identified in the south of England in September 2020. This variant has a mutation in the RBD of the spike protein at position 501, where the amino acid asparagine (N) has been replaced with tyrosine (Y), hence the mutation is referred to as N501Y. The B.1.1.7 variant is associated with more efficient and rapid transmission and increased risk of death compared with other variants. The B1.1.351 variant was initially identified in South Africa in October 2020. This variant has multiple mutations in the spike protein, including K417N, E484K, and N501Y. The mutation E484K has been shown to affect neutralization by polyclonal and monoclonal antibodies. The B.1.1.28 variant, specifically the P.1 branch of the lineage, was initially identified in January 2021 in travelers from Brazil who arrived in Japan. The P.1 lineage contains three mutations in the spike protein RBD, including K417T, E484K, and N501Y. The mutations increase the transmissibility and antigenic profile of SARS-CoV-2.

As used herein, the 51 subunit of the spike protein of SARS-CoV-2 contains immunodominant T cell epitopes YNYLYRLF (SEQ ID NO:9), VVLSFELL (SEQ ID NO:10), and VNFNFNGL (SEQ ID NO:11) within the receptor binding domain region. The VVLSFELL (SEQ ID NO:10) and VNFNFNGL (SEQ ID NO:11) epitopes were previously also identified in mouse studies of SARS-CoV. It would be understood by persons skilled in the art that these epitopes conserved between SARS-CoV and SARS-CoV-2 may indicate that vaccines directed to raise an immune response in SARS-CoV-2 may be cross-reactive towards SARS-CoV.

The genetic sequence of human coronavirus SARS-CoV-2 strains/isolates is made available via the Global Initiative on Sharing All Influenza Data (GISAID) and includes genomic sequence data from various strains/isolates of SARS-CoV-2 including, for example, Wuhan/IVDC-HB-01/2019 (GISAID accession ID: EPI_ISL_402119-121). The genome sequences of SARS-CoV-2 are crucial to design and evaluate potential intervention options, such as vaccines against COVID19.

The present invention provides a composition comprising an attenuated poxvirus for expressing heterologous coronavirus antigens which can be used as a vaccine for inducing an immune response and/or a neutralizing antibody response against coronavirus infection. The terms “attenuation”, “attenuated” and the like, as used herein, mean a reduction of viral vector virulence. Virulence is typically defined as the ability of a virus to cause disease in a particular host. For instance, a poxvirus that is unable to produce infectious viruses may initially infect cells but is substantially unable to replicate itself fully or propagate within the host or host cell or cause a disease or condition. This is desirable, as the poxvirus vector can deliver nucleic acid to the host or host cell, but typically does not harm the host or host cell.

The poxvirus family comprises two subfamilies, the Chordopoxvirinae and the Entomopoxvirinae. The Chordopoxvirinae comprises eight genera including the Orthopoxviridae comprising species which infect man while the Entomopoxvirinae infect insects. The Orthopoxviridae includes for example, variola virus which is the causative agent of smallpox, cowpox virus which formed the original smallpox vaccine reported by Jenner in 1796, and vaccinia virus which has been used as a second generation smallpox vaccine. The Avipoxviridae virus comprises species that infect birds, such as fowlpox and canarypox viruses. In addition to their use as antigens in smallpox vaccines, there is much interest in the use of recombinant vaccinia-based viruses and avipox viruses as vectors for delivering and/or expressing heterologous genes of interest. As intracytoplasmic vectors, the Orthopoxviridae are able to deliver foreign antigens to the host cytoplasm and antigen processing pathways that process antigens to peptides for presentation on the cell surface. Such vectors expressing foreign antigens are suitable for use in gene therapy and the development of vaccines for a wide range of conditions and diseases.

The poxviruses constitute a large family of viruses characterized by a large, linear dsDNA genome, a cytoplasmic site of propagation and a complex virion morphology. Vaccinia virus is the representative virus of this group of viruses and one of the most studied in terms of viral morphogenesis. Vaccinia virus various appear as “brick shaped” or “ovoid” membrane-bound particles with a complex internal structure featuring a walled, biconcave core flanked by “lateral bodies”. The virion assembly pathway involves a fabrication of membrane containing crescents which develop into immature virions (IVs), and then evolve into mature virions (MVs). Over 70 specific gene products are contained within the vaccinia virus virion, where the effects of mutations in over 50 specific genes on vaccinia virus assembly are now described.

Suitable attenuated poxviruses would be known to persons skilled in the art. Illustrative examples include attenuated Modified Vaccinia Ankara (MVA), NYVAC, avipox, canarypox and fowlpox.

In an embodiment disclosed herein, the attenuated poxvirus is an attenuated vaccinia virus. Illustrative examples of vaccinia virus strains include MVA, NYVAC, Copenhagen (COP), Western Reserve (WR), NYCBH, Wyeth strain, ACAM2000, LC16m8 and Connaught Laboratories (CL).

As used herein, the term “Sementis Copenhagen Vector” or “SCV” refers to a vaccinia-virus based, multiplication-defective, vaccine vector technology platform that allows manufacture in modified CHO cells.

It would be understood by persons skilled in the art that other orthopoxvirus strains may be modified to produce an attenuated poxvirus. In an illustrative example, an attenuated poxvirus can be produced by modifying (e.g., deleting, substituting or otherwise disrupting the function of) a gene from the poxvirus genome that encodes an endogenous essential assembly or maturation protein. Thus, in an embodiment disclosed herein, the attenuated poxvirus is a modified orthopoxvirus, wherein the modification comprises deletion of a gene encoding an endogenous essential assembly or maturation protein.

In an embodiment, the attenuated poxvirus is a modified vaccinia virus wherein the modification comprises deletion of a gene of the vaccinia virus genome encoding (or otherwise disruption of the function of) an endogenous assembly or maturation protein and wherein the modification transforms a vaccinia vectors which propagates (or which may propagate) in a host cell (e.g., a human cell) into an attenuated vaccinia vector which is substantially non-replicative in the host cell.

In an embodiment, the essential endogenous assembly or maturation gene is selected from the group comprising of COP-A2.5L, COP-A3L, COP-A4L, COP-A7L, COP-A8R, COP-A9L, COP-A10L, COP-A11R, COP-A12L, COP-A13L, COP-A14L, COP-A14.5L, COP-A15L, COP-A16L, COP-A17L, COP-A21 L, COP-A22R, COP-A26L, COP-A27L, COP-A28L, COP-A30L, COP-A32L, COP-D2L, COP-D3R, COP-D6R, COP-D8L, COP-D13L, COP-D14L, COP-E8R, COP-E10R, COP-E11L, COP-F10L, COP-F17R, COP-G1L, COP-G3L, COP-G4L, COP-G5R, COP-G7L, COP-H1L, COP-H2R, COP-H3L, COP-H4L, COP-H5R, COP-H6R, COP-I1 L, COP-I2L, COP-I6L, COP-17L, COP-I8R, COP-J1R, COP-J4R, COP-J6R, COP-L1R, COP-L3L, COP-L4R and COP-L5R.

It would be understood by persons skilled in the art that other orthopoxvirus strains may be modified to alter the immunogenicity of the poxvirus. In an illustrative example, a poxvirus with enhanced immunogenicity can be produced by deleting a gene from the poxvirus genome that encodes an immunomodulatory protein. Thus, in an embodiment disclosed herein, the attenuated poxvirus is a modified orthopoxvirus, wherein the modification comprises deletion of one or more genes encoding immunomodulatory protein/s.

In an embodiment, the immune modulatory gene or genes include those selected from the group comprising of COP-C23L, COP-B29R, COP-C3L, COP-N1 L, COP-A35R, COP-A39R, COP-A41 L, COP-A44R, COP-A46R, COP-B7R, COP-B8R, COP-B13R, COP-B16R, and COP-B19R.

It would be understood by persons skilled in the art that other orthopoxvirus strains may be modified to incorporate heterologous DNA sequences that can be stably inserted into the vaccinia genome, especially in intergenic regions, without disruption or alteration to the coding sequence, thereby retaining the typical characteristics and gene expression of the virus.

In an embodiment, the attenuated poxvirus is a modified vaccinia virus wherein the modification comprises exogenous DNA sequence, for example a DNA sequence derived from Coronavirus strains, inserted into an intergenic region of the viral genome, wherein the intergenic region is in turn, located between or are flanked by two adjacent open reading frames (ORF) of the vaccinia genome, and wherein the open reading frames correspond to conserved genes.

In an embodiment, the intergenic region or regions in between two adjacent ORFs wherein heterologous DNA sequences can be inserted include those selected from the group comprising of 001L-002L, 002L-003L, 005R-006R, 006L-007R, 007R-008L, 008L-009L, 017L-018L, 018L-019L, 019L-020L, 020L-021 L, 023L-024L, 024L-025L, 025L-026L, 028R-029L, 030L-031 L, 031L-032L, 032L-033L, 035L-036L, 036L-037L, 037L-038L, 039L-040L, 043L-044L, 044L-045L, 046L-047R, 049L-050L, 050L-051 L, 051 L-052R, 052R-053R, 053R-054R, 054R-055R, 055R-056L, 061 L-062L, 064L-065L, 065L-066L, 066L-067L, 077L-078R, 078R-079R, 080R-081R, 081R-082L, 082L-083R, 085R-086R, 086R-087R, 088R-089L, 089L-090R, 092R-093L, 094L-095R, 096R-097R, 097R-098R, 101R-102R, 103R-104R, 105L-106R, 107R-108L, 108L-109L, 109L-110L, 110L-111L, 113L-114L, 114L-115L, 115L-116R, 117L-118L, 118L-119R, 122R-123L, 123L-124L, 124L-125L, 125L-126L, 133R-134R, 134R-135R, 136L-137L, 137L-138L, 141 L-142R, 143L-144R, 144R-145R, 145R-146R, 146R-147R, 147R-148R, 148R-149L, 152R-153L, 153L-154R, 154R-155R, 156R-157L, 157L-158R, 159R-160L, 160L-161R, 162R-163R, 163R-164R, 164R-165R, 165R-166R, 166R-167R, 167R-168R, 170R-171R, 173R-174R, 175R-176R, 176R-177R, 178R-179R, 179R-180R, 180R-181R, 183R-184R, 184R-185L, 185L-186R, 186R-187R, 187R-188R, 188R-189R, 189R-190R, 192R-193R (also see PCT/EP03/05045).

According to the old nomenclature, ORF 006L corresponds to Cl 0L, 019L corresponds to C6L, 020L to N1L, 021L to N2L, 023L to K2L, 028R to K7R, 029L to F1L, 037L to F8L, 045L to F15L, 050L to E3L, 052R to E5R, 054R to E7R, 055R to E8R, 056L to E9L 062L to I1 L, 064L to I4L, 065L to I5L, 081R to L2R, 082L to L3L, 086R to J2R, 087 to J3R, 088R to J4R, 089L to J5L, 092R to H2R, 095R to H5R, 107R to D10R, 108L to D11L, 122R to A11R, 123L to A12L, 125L to A14L, 126L to A15L, 135R to A24R, 136L to A25L, 137L to A26L, 141L to A30L, 148R to A37R, 149L to A38 L, 152R to A40R, 153L to A41L, 154R to A42R, 157L to A44L, 159R to A46R, 160L to A47L, 165R to A56R, 166R to A57R, 167R to B1R, 170R to B3R, 176R to B8R, 180R to B12R, 184R to B16R, 185L to B17L, and 187R to B19R.

In an embodiment, the intergenic region or regions in between two adjacent ORFs wherein heterologous DNA sequences can be inserted include those selected from the group comprising of F9L-F10L, F12L-F13L, F17R-E1 L, E1L-E2L, E8R-E9L, E9L-E10R, I1L-12L, 12L-I3L, 15L-I6L, 16L-17L, 17L-I8R, I8R-G1L, G1L-G3L, G3L-G2R, G2R-G4L, G4L-G5R, G5R-G5.5R, G5.5R-G6R, G6R-G7L, G7L-G8R, G8R-G9R, G9R-L1R, L1R-L2R, L2R-L3L, L3L-L4R, L4R-L5R, L5R-J1R, J3R-J4R, J4R-J5L, J5L-J6R, J6R-H1L, H1L-H2R, H2R-H3L, H3L-H4L, H4L-H5R, H5R-H6R, H6R-H7R, H7R-D1R, D1R-D2L, D2L-D3R, D3R-D4R, D4R-D5R, D5R-D6R, D6R-D7R, D9R-D10R, D10R-D11L, D11L-D12L, D12L-D13L, D13L-A1L, A1L-A2L, A2L-A2.5L, A2.5L-A3L, A3L-A4L, A4L-ASR, A5R-A6L, A6L-A7L, A7L-A8R, A8R-A9L, A9L-A10L, A10L-A11R, A11R-A12L, A12L-A13L, A13L-A14L, A14L-A14.5L, A14.5L-A15L, A15L-A16L, A16L-A17L, A17L-A18R, A18R-A19L, A19L-A21L, A21L-A20R, A20R-A22R, A22R-A23R, A23R-A24R, A28L-A29L, A29L-A30L (also see PCT/IB2007/004575).

In an embodiment the modification comprises deletion of the A41L gene.

In an embodiment the modification comprises deletion of the A41L gene and/or the D13L gene.

In an embodiment the modification comprises deletion of the A41L gene and/or the D13L gene and/or the B7R-B8R genes.

In an embodiment, the modification comprises deletion of the A41L gene and/or the D13L gene and/or the B7R-B8R genes, and/or the C3L gene, and/or the A39R gene.

It would be understood by persons skilled in the art that deletion of the A41 L gene and/or the D13L gene and/or the B7R-B8R genes, and/or the C3L gene, and/or the A39R gene, imparts advantageous characteristics to the poxvirus such as attenuation and increased immunogenicity.

In an embodiment, the modification comprises insertion of heterologous DNA sequence in the intergenic region located between J2R and J3R genes.

It would be understood by persons skilled in the art that insertion of heterologous DNA sequence in the intergenic region does not disrupt nor alter the coding sequence of the virus.

In an embodiment, the recombinant SCV vector expresses one or more structural proteins and non-structural proteins that assemble into VLPs.

In an embodiment, the SARS-CoV-2 antigens assemble into virus-like particles (VLPs) when expressed.

In an embodiment, the vector expresses proteins that form VLPs and generate an immune response to a SARS-CoV-2 antigen or immunogenic fragment thereof.

In exemplary embodiments, the immune responses are long-lasting and durable so that repeated boosters are not required, but in one or more embodiment/s, one or more administrations of the compositions provided herein are provided to boost the initial primed immune response.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter.

As used herein, the use of spike protein includes engineered variants directed to stabilize the spike protein in its prefusion conformation, preventing structural rearrangement, and exposing antigenically preferable surfaces in order to elicit superior immune responses. These modifications include, but is not limited to, introducing stabilizing mutations and using molecular clamps.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a synthetic early/late promoter, the membrane polypeptide of SARS-CoV-2 or an immunogenic or functional part thereof under transcriptional control of a fowlpox early/late promoter, the nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the spike polypeptide of SARS-CoV-2 or an immunogenic or functional part thereof under transcriptional control of a native early/late promoter, the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter, and the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter.

In an embodiment, the present invention provides a composition for raising an immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the S1 receptor-binding domain subunit of the spike polypeptide of SARS-CoV-2 under transcriptional control of a synthetic early/late promoter, and the membrane polypeptide of SARS-CoV-2 or an immunogenic or functional part thereof under transcriptional control of a fowlpox early/late promoter, and nucleocapsid polypeptide of SARS-CoV-2 or immunogenic or functional part thereof under transcriptional control of a synthetic early/late promoter, and the envelope polypeptide of SARS-CoV-2 or an immunogenic or functional part thereof under transcriptional control of a synthetic early/late promoter.

In an embodiment, the present invention provides a method of inducing a protective immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the membrane polypeptide of SARS-CoV-2 or an immunogenic or functional part thereof under transcriptional control of a fowlpox early/late promoter.

In an embodiment, the present invention provides a method of inducing a protective immune response in animal which prevents or decreases the risk of SARS-CoV-2 infection and/or decrease the severity of COVID-19 disease, the composition comprising an attenuated poxvirus, and especially a vaccinia virus, wherein the poxvirus genome has been modified and comprises a nucleic acid sequence encoding the nucleocapsid polypeptide of SARS-CoV-2 or an immunogenic or functional part thereof under transcriptional control of a synthetic early/late promoter.

In an embodiment, the present invention provides a method of inducing a protective immune response in a subject against SARS-CoV-2 virus infection the method comprising administering to the subject a mixed composition comprising equal amounts of the composition comprising an attenuated poxvirus, wherein the poxvirus genome has been modified and comprises a nucleic acid sequence encoding the spike polypeptide of SARS-CoV-2 or an immunogenic or functional part thereof under transcriptional control of a synthetic early/late promoter and the composition comprising an attenuated poxvirus, wherein the poxvirus genome has been modified and comprises a nucleic acid sequence encoding the membrane and nucleocapsid polypeptides of SARS-CoV-2 or an immunogenic or functional part or parts thereof.

In an embodiment, the present invention provides a method of inducing a protective immune response in a subject against SARS-CoV-2 virus infection the method comprising administering to the subject a mixed composition comprising equal amounts of the composition comprising an attenuated poxvirus, wherein the poxvirus genome has been modified and comprises a nucleic acid sequence encoding the spike polypeptide of SARS-CoV-2 or an immunogenic or functional part thereof under transcriptional control of a native early/late promoter and the composition comprising an attenuated poxvirus, wherein the poxvirus genome has been modified and comprises a nucleic acid sequence encoding the membrane and nucleocapsid polypeptides of SARS-CoV-2 or an immunogenic or functional part or parts thereof.

In a first aspect, the present invention provides a method of inducing a protective immune response in a subject against SARS-CoV-2 virus infection the method comprising administering to the subject the composition as any of the above.

In a second aspect, the present invention provides a composition for raising an immune response in animal which decreases the risk of SARS-CoV-2 infection by resembling SARS-CoV-2 virus-like particles.

In a third aspect, the present invention provides a composition for raising an immune response in animal which decreases the risk of SARS-CoV-2 infection and any other infection caused by coronaviruses with genetic similarity to SARS-CoV-2, the composition comprising an attenuated poxvirus, wherein the poxvirus genome comprises a nucleic acid sequence encoding the spike polypeptide of SARS-CoV-2 or an immunogenic or functional part thereof, and/or the membrane and nucleocapsid polypeptides of SARS-CoV-2 or an immunogenic or functional part thereof, and/or the envelope polypeptide of SARS-CoV-2 or an immunogenic or functional part thereof.

In a fourth aspect, the present invention provides the use of the composition of the embodiments as contemplated herein, in the preparation of a medicament for inducing a neutralizing antibody response and/or protective immune response in a subject against a coronavirus infection.

Immunogenicity may be achieved by expressing the SARS-CoV-2 spike polypeptide or the S1 receptor binding domain subunit of the spike polypeptide from a poxvirus vector. Historically, the spike protein of coronaviruses such as SARS-CoV or MERS-CoV have been found immunogenic, eliciting humoral immune responses including neutralizing antibodies that inhibit virus entry into host cells as well as cell-mediated immune responses. Immunogenicity may also be achieved by inducing spike-specific T cell responses. Spike-specific cellular and humoral responses induced by the SCV-COVID vaccine against the SARS-CoV-2 virus complemented with the Th1-biased production of antibodies induced by the poxvirus vector may provide prophylactic protection against SARS-CoV-2 infection.

Immunogenicity may be achieved by expressing the SARS-CoV-2 membrane protein polypeptide from a poxvirus vector. In SARS-CoV, the membrane protein has been shown to be abundant on the viral surface; moreover, when used for immunization in patients with SARS, the membrane protein induced high titres of neutralizing antibodies. Immunogenic and structural analyses demonstrated that a T-cell epitope cluster capable of triggering a robust cellular immune response exists in the membrane protein. As the membrane protein is also highly conserved in many virus species, it is a good antigen candidate for inducing immune response against SARS-CoV-2. Membrane-specific cellular and humoral responses induced by the SCV-COVID vaccine against the SARS-CoV-2 virus complemented with the Th1-biased production of antibodies induced by the poxvirus vector may provide prophylactic protection against SARS-CoV-2 infection.

Immunogenicity may be achieved by expressing the SARS-CoV-2 nucleocapsid protein polypeptide from a poxvirus vector. It was recently discovered that SARS-CoV-2 infection leads to production of antibodies that are mostly directed to the nucleocapsid antigen. However, N antibodies have been overlooked as N protein antibodies cannot block virus entry and as such are considered ‘non-neutralizing’ antibodies. Therefore, anti-N antibodies cannot be measured by neutralization assays that are currently in use to assess humoral immunity. Recent studies have shown that anti-N antibodies that get inside cells are recognized by an antibody receptor TRIM21, which then shreds the associated N protein. N protein epitopes are then displayed for detection by T cells. As this immune response mechanism involves T cells that will eventually mediate immunological memory, antibodies against the nucleocapsid protein might stimulate long-term protection against future infection. Nucleocapsid-specific cellular and humoral responses induced by the SCV-COVID vaccine against the SARS-CoV-2 virus complemented with the Th1-biased production of antibodies induced by the poxvirus vector may provide prophylactic protection against SARS-CoV-2 infection.

Immunogenicity may be achieved by simultaneously expressing the SARS-CoV-2 spike protein, or parts thereof, membrane protein polypeptide, nucleocapsid protein polypeptide, and/or envelope protein polypeptide within a poxvirus vector. The combined immunogenicity of the structural proteins may bring about a more robust antigen-specific immune response. Furthermore, the presence of S, M, and N and/or E polypeptides may lead to the formation of an authentic virus like particle (VLP), empty virus shells that mimic the coronavirus structure but lacks the genetic material to be infectious. Antigen- and VLP-specific cellular and humoral response induced by the SCV-COVID vaccine against the SARS-CoV-2 virus complemented with the Th1-biased production of antibodies induced by the poxvirus vector may provide prophylactic protection against COVID-19. VLP-specific cellular and humoral response induced by the SCV-COVID vaccine against the SARS-CoV-2 virus complemented with the Th1-biased production of antibodies induced by the poxvirus vector may provide prophylactic protection against COVID-19.

In preferred forms of the current invention the attenuated poxvirus is selected from the group consisting of vaccinia virus, NYVAC, and SCV. It is preferred that the attenuated poxvirus is a modified orthopoxvirus, wherein the modification comprises deletion of a gene encoding an endogenous essential assembly or maturation protein. It is further preferred that the modification comprises deletion of the D13L gene and preferably further comprises the deletion of the K1L gene.

The various embodiments enabled herein are further described by the following non-limiting examples.

### Example 1

### Construction of Vaccines

To construct SCV-COVID19 vaccines, early transcription termination signals within antigen coding sequences were removed using silent mutations and expression cassettes for antigenic transgenes were synthesized de novo or constructed by PCR from synthesized cassettes. Each cassette consists of the transgene flanked by the necessary control elements of a poxvirus promoter and Kozak sequence upstream, and poxvirus early transcription termination signal downstream to enable gene expression. Flanking endonuclease recognition sites may also have been included to enable molecular manipulation. Specific examples of expression cassettes are shown for the SARS-CoV-2 spike polypeptide with a synthetic early/late promoter (FIG. 1A), SARS-CoV-2 spike polypeptide with a native early/late promoter (FIG. 1B), 51 receptor-binding domain subunit of the SARS-CoV-2 spike polypeptide with a synthetic early/late promoter (FIG. 1C) membrane polypeptide with a fowlpox early/late promoter (FIG. 1D), nucleocapsid polypeptide with a synthetic early/late promoter (FIG. 1E), membrane polypeptide with a fowlpox early/late promoter and nucleocapsid polypeptide with a synthetic early/late promoter (FIG. 1F), envelope polypeptide with a synthetic early/late promoter (FIG. 1G).

Transgene expression cassettes were then inserted into appropriate homologous recombination (HR) plasmids able to be propagated in bacteria using standard molecular biology methods. The HR plasmid contains the HR cassette consisting of flanking recombination arms (F1 and F2) homologous to poxvirus genome sites between which the transgene is located. Homologous recombination sites relative to the vaccinia-COP genome are indicated in FIG. 2. The transgene expression cassette is inserted between the recombination arms, adjacent to additional poxvirus expression cassettes containing genes to enable positive selection of the new recombinant virus (e.g. CP77, Zeocin resistance or other drug selection combined with a fluorescent reporter protein such as green or blue fluorescent protein (GFP or BFP). Selection genes are flanked by 150 bp of identical, non-coding DNA sequence to enable selection gene deletion once the parent virus has been eliminated. To prepare the HR plasmid for virus construction, restriction endonuclease digestion (e.g. Not I) is used to release the HR cassette. Specific examples of HR cassettes are shown for:
The SARS-CoV-2 spike polypeptide under transcriptional control of synthetic early/late promoter expression cassette flanked by F1 and F2 recombination arms that were homologous to sequences flanking the vaccinia A41 L ORF (FIG. 3A; SEQ ID NO:1),
The SARS-CoV-2 spike polypeptide under transcriptional control of native early/late promoter expression cassette flanked by F1 and F2 recombination arms that were homologous to sequences flanking the vaccinia A41 L ORF (FIG. 3B; SEQ ID NO:2),
The S1 subunit of the SARS-CoV-2 spike polypeptide expression cassette flanked by F1 and F2 recombination arms that were homologous to sequences flanking the vaccinia A41 L ORF (FIG. 3C; SEQ ID NO:3),
The SARS-CoV-2 membrane and nucleocapsid proteins expression cassette flanked by left and right combination arms that were homologous to sequences flanking the vaccinia D13L ORF (FIG. 3D, SEQ. NO. 4) or the vaccinia J2R and J3R ORFs enabling insertion between J2R and J3R (FIG. 2E, SEQ ID NO:5),
The SARS-CoV-2 membrane protein expression cassette flanked by left and right combination arms that were homologous to sequences flanking the vaccinia C3L ORF (FIG. 3F; SEQ ID NO:6),
The SARS-CoV-2 nucleocapsid protein expression cassette flanked by left and right combination arms that were homologous to sequences flanking the vaccinia D13L ORF (FIG. 3G; SEQ ID NO:7).
The SARS-CoV-2 envelope protein expression cassette flanked by left and right combination arms that were homologous to sequences flanking the vaccinia B7/B8R ORF (FIG. 3H; SEQ ID NO:8).

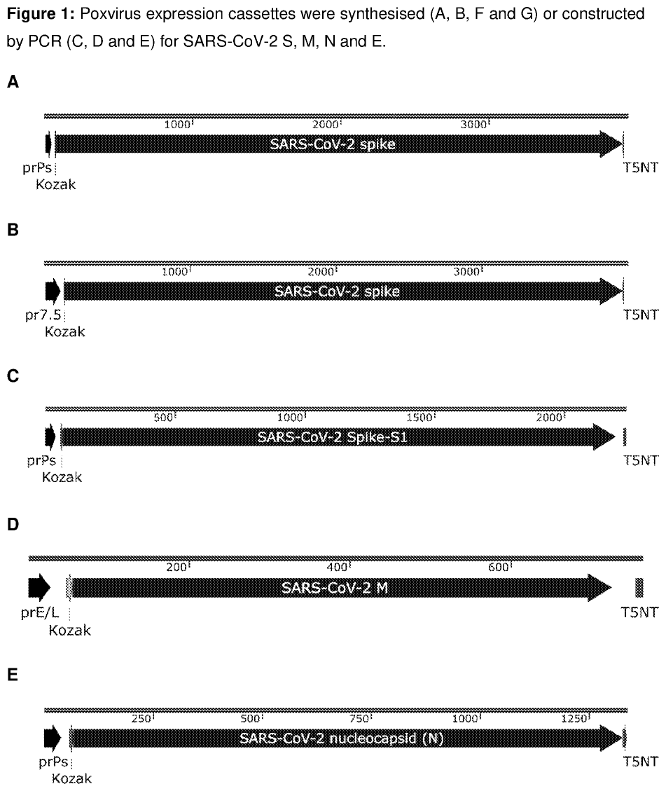

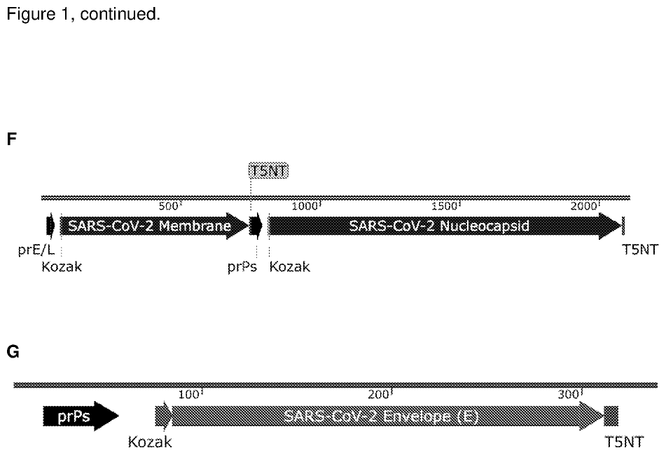

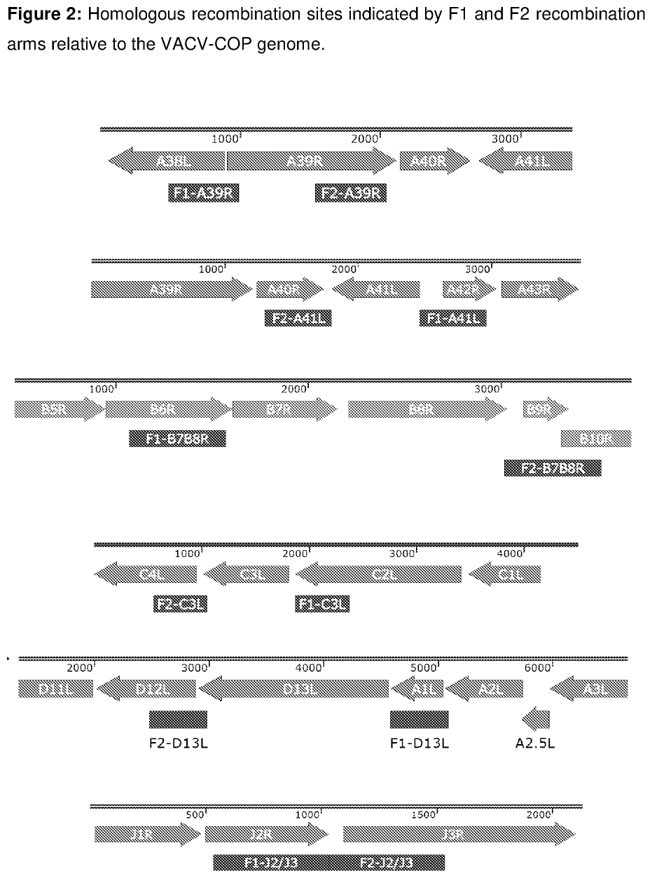

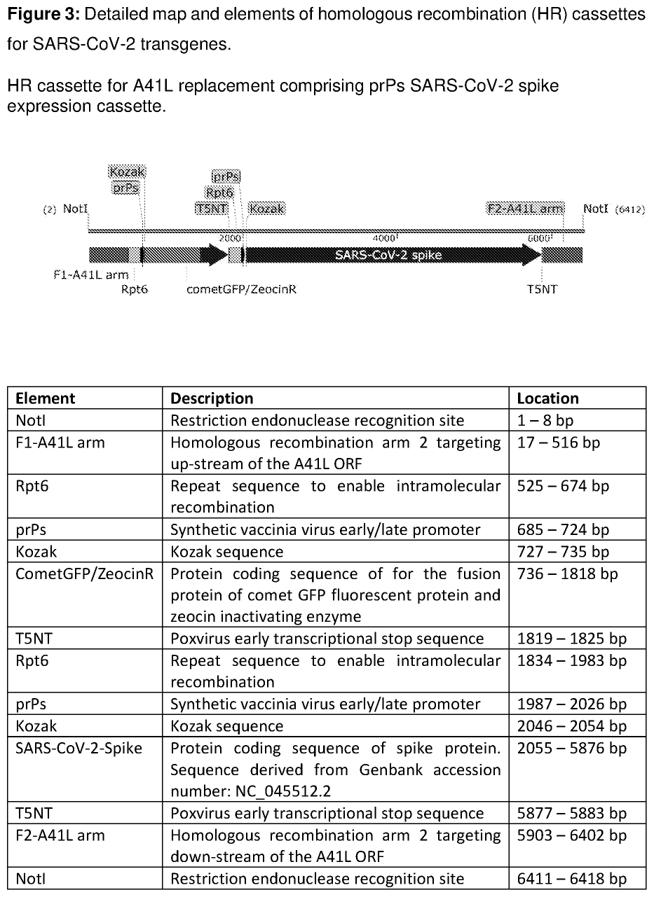

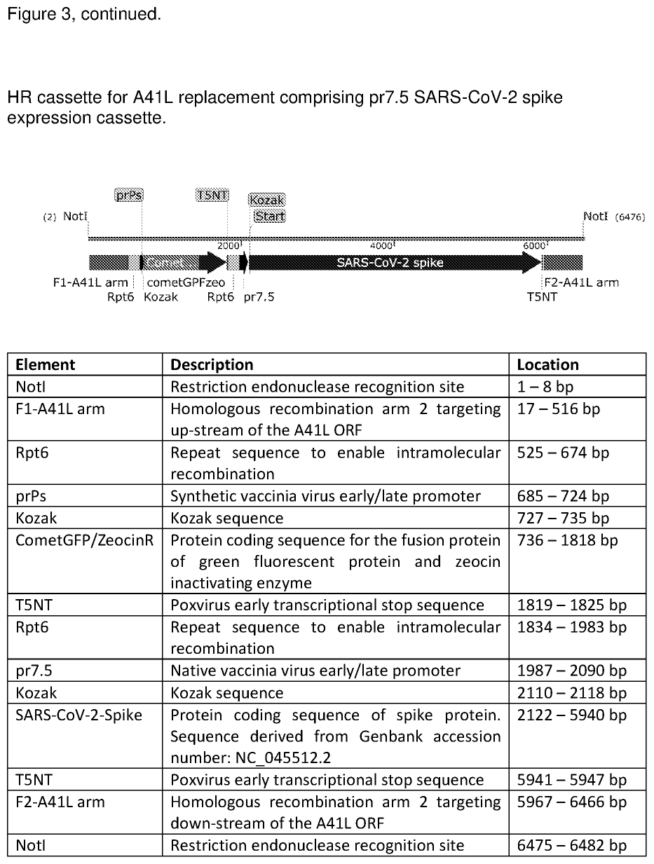

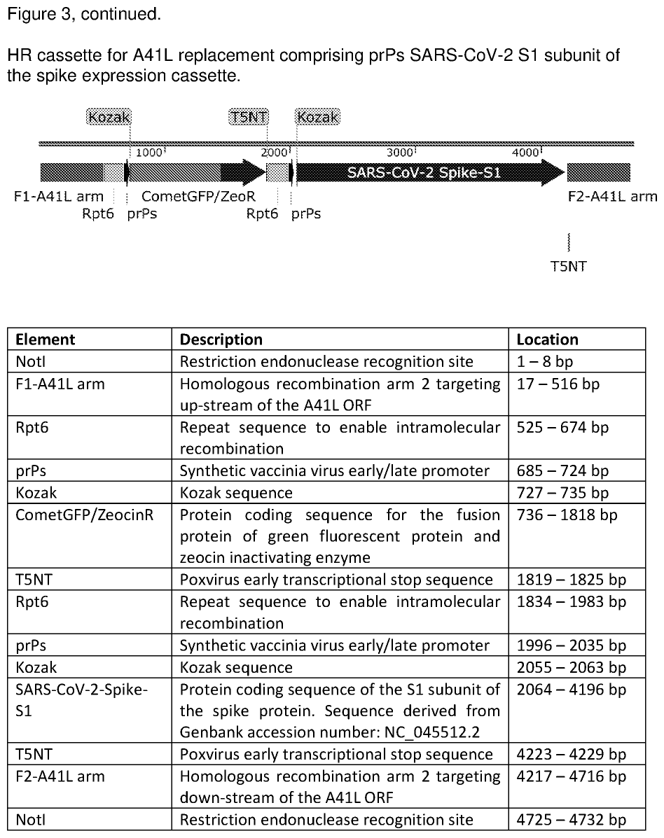

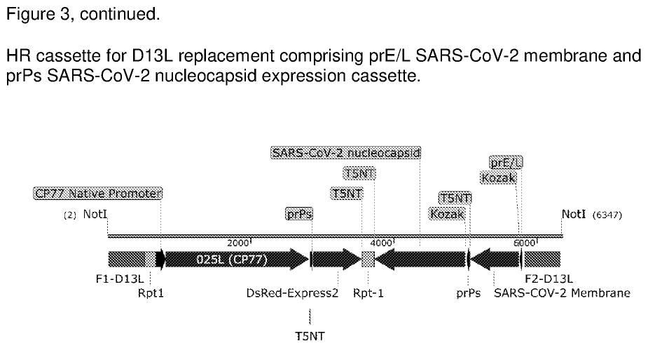

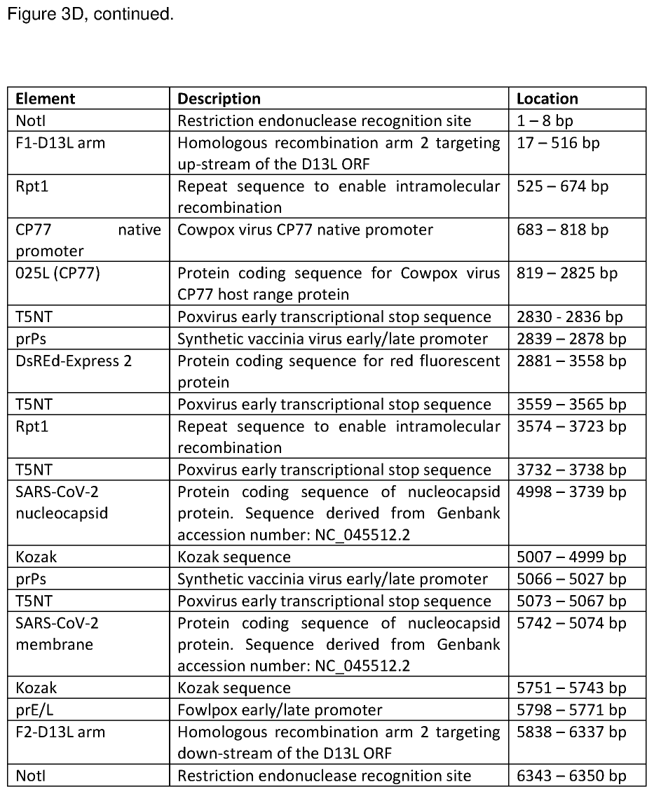

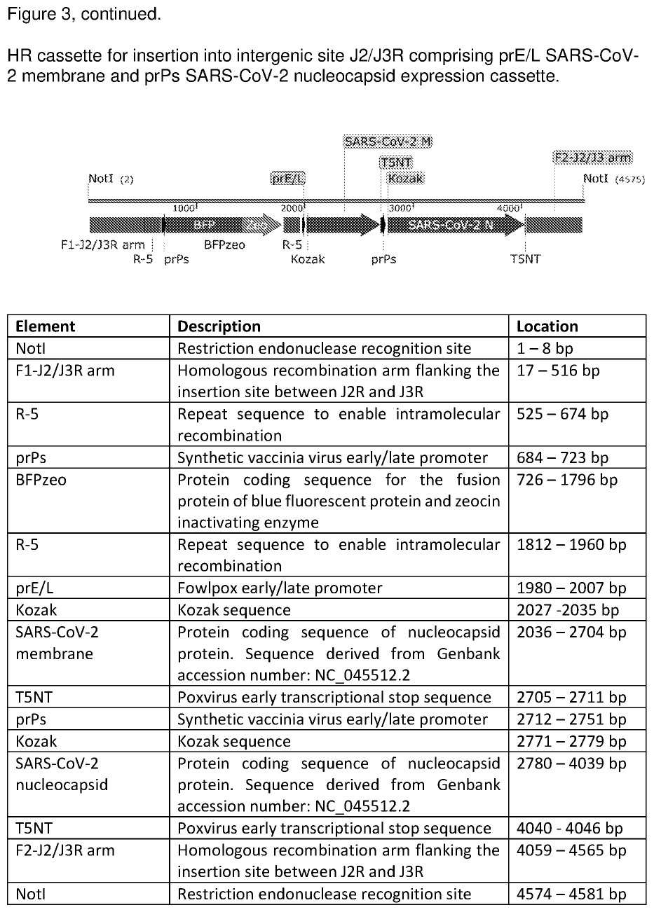

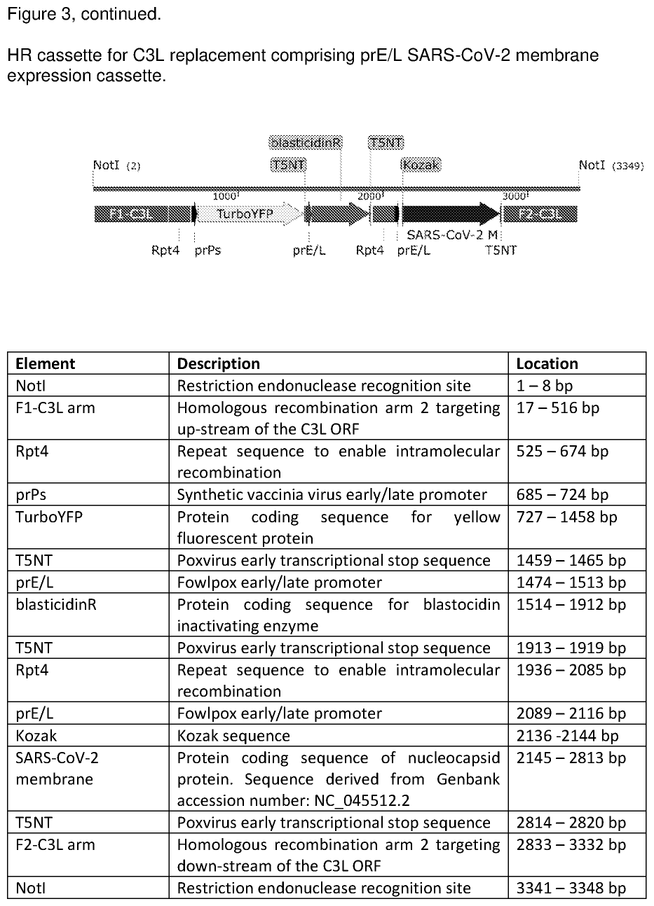

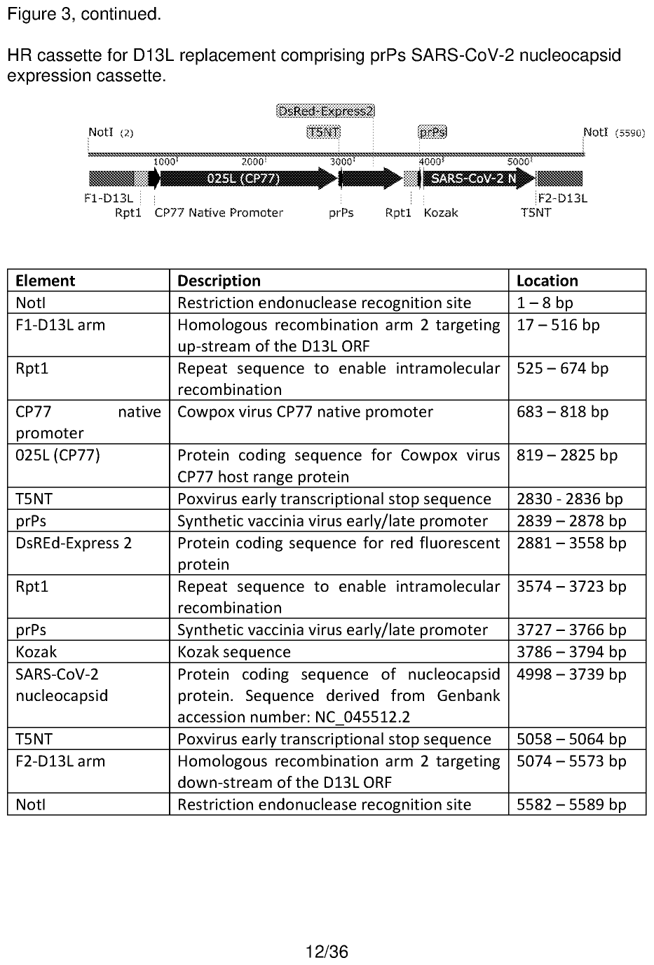

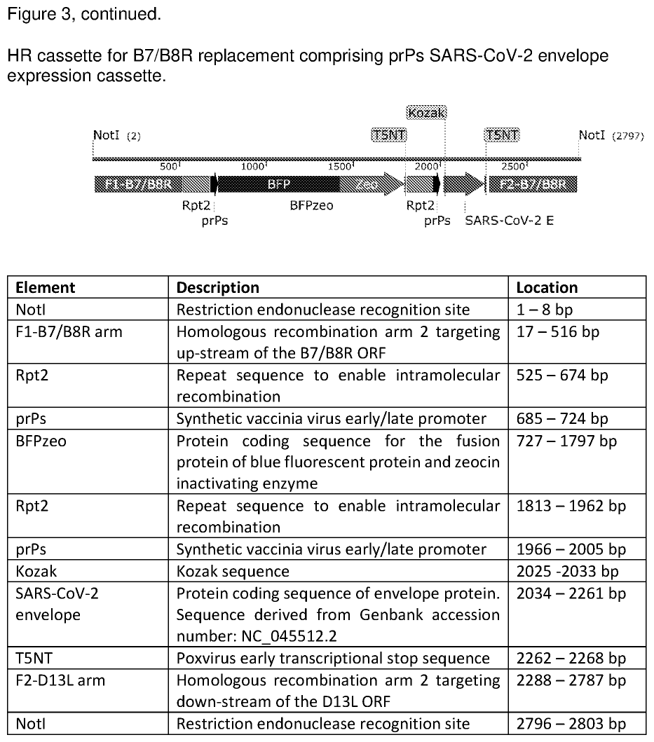

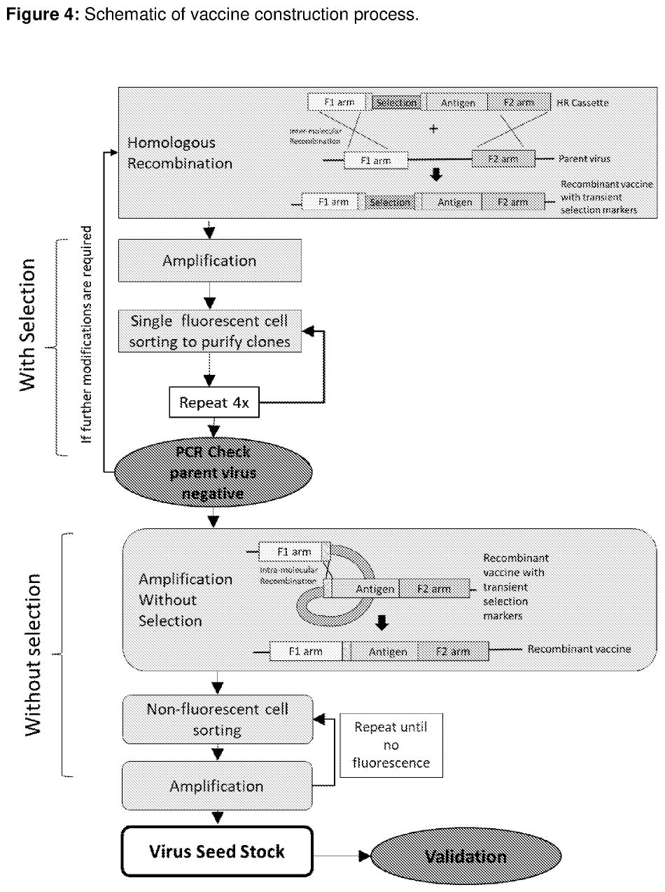

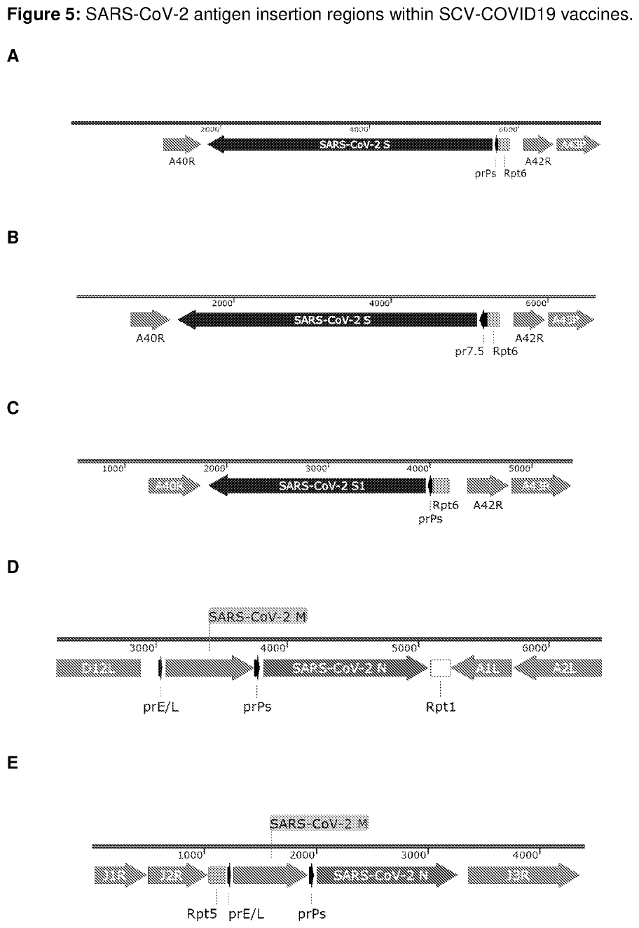

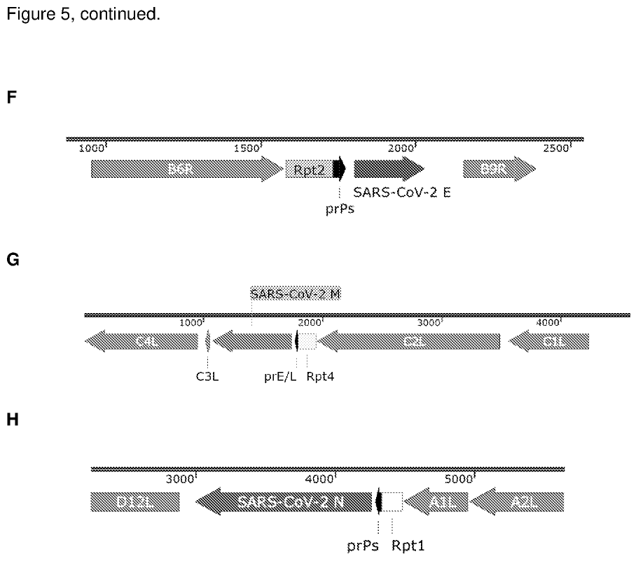

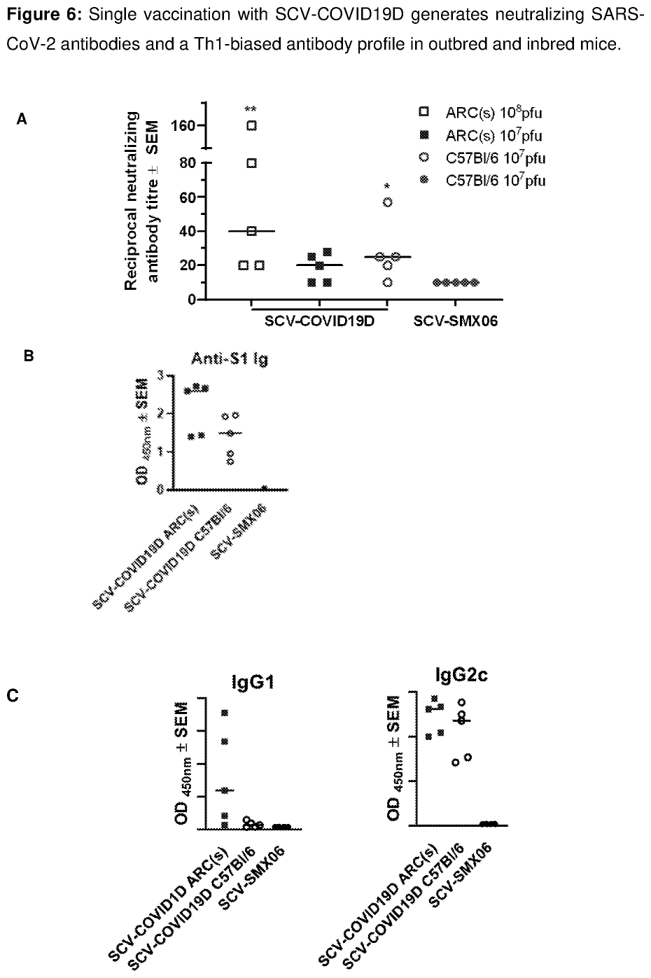

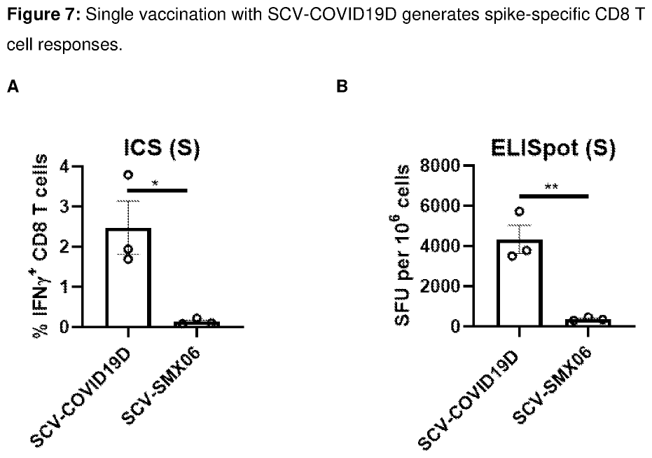

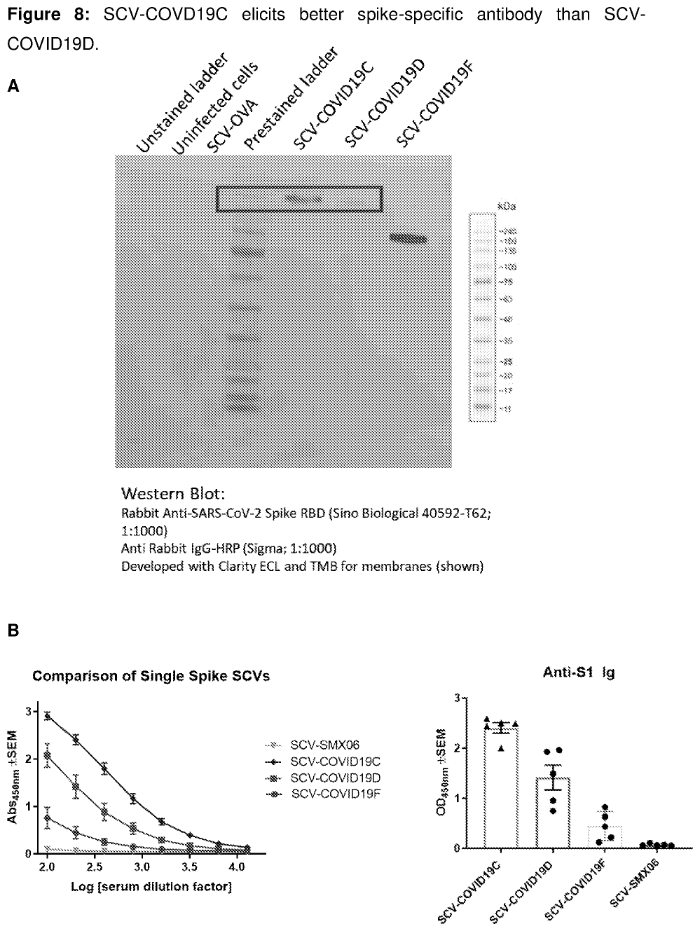

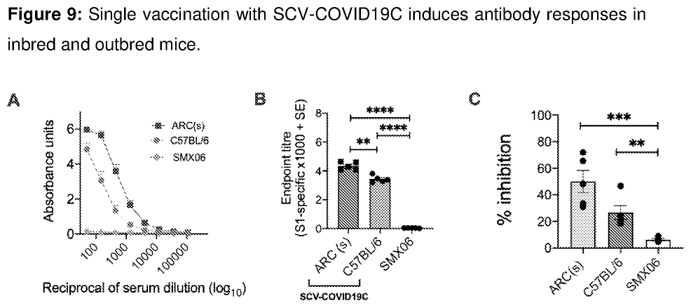

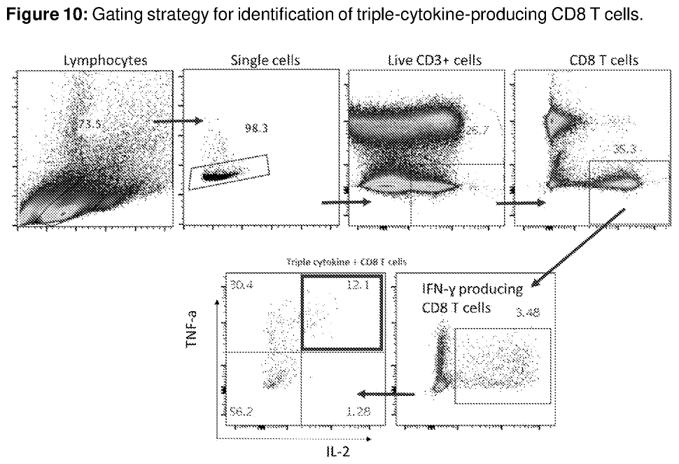

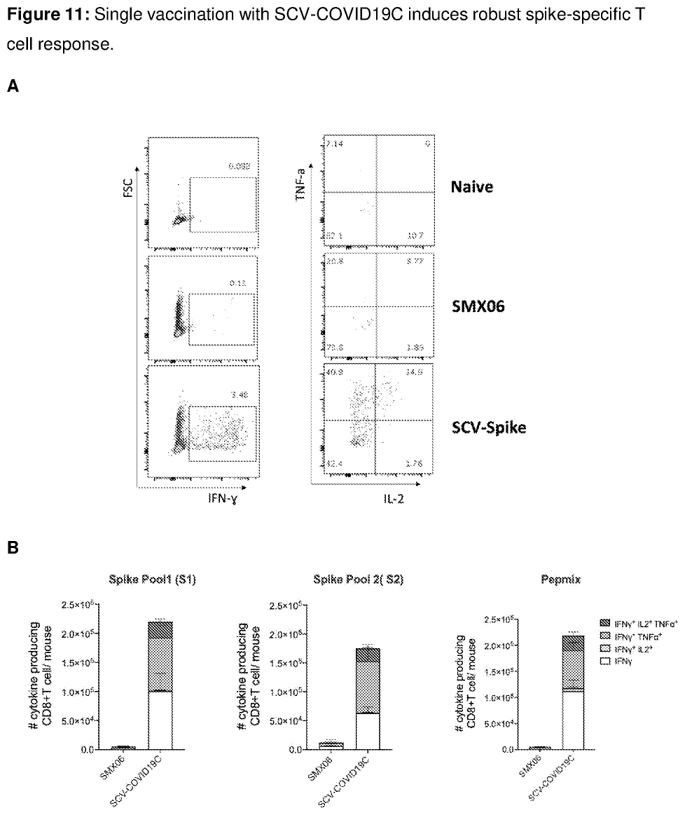

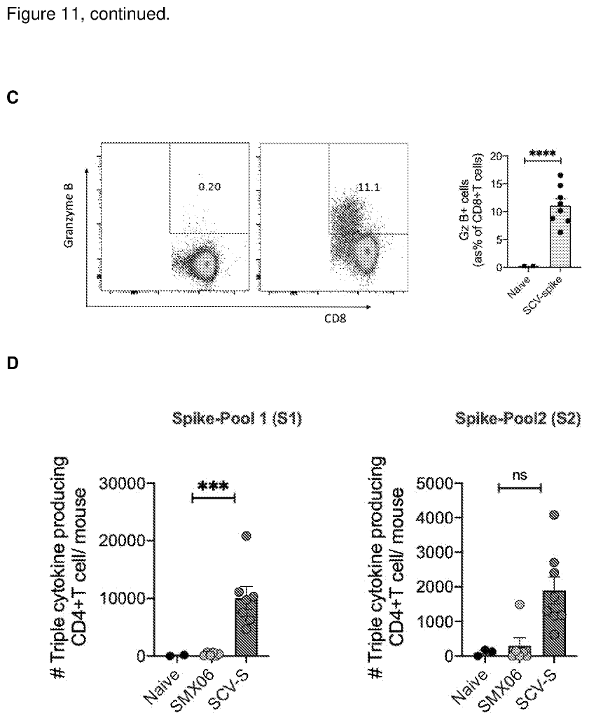

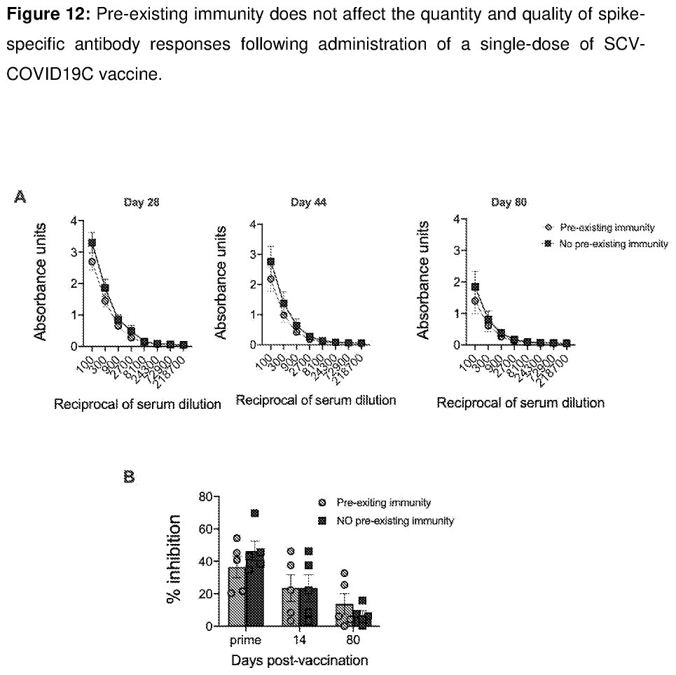

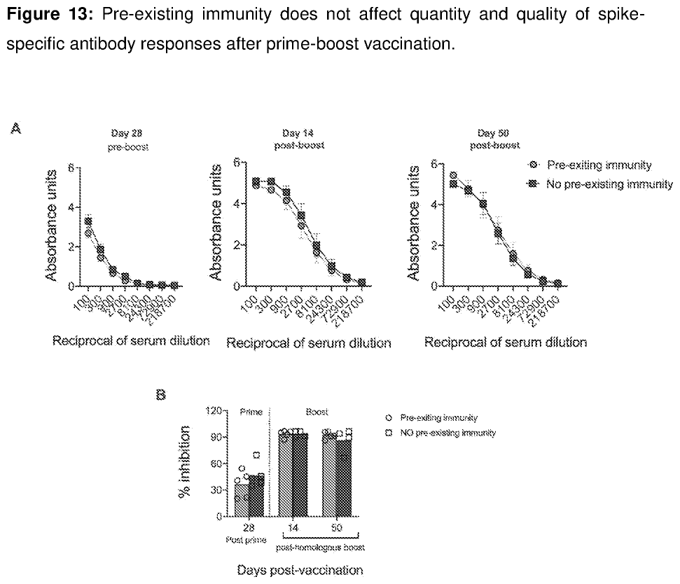

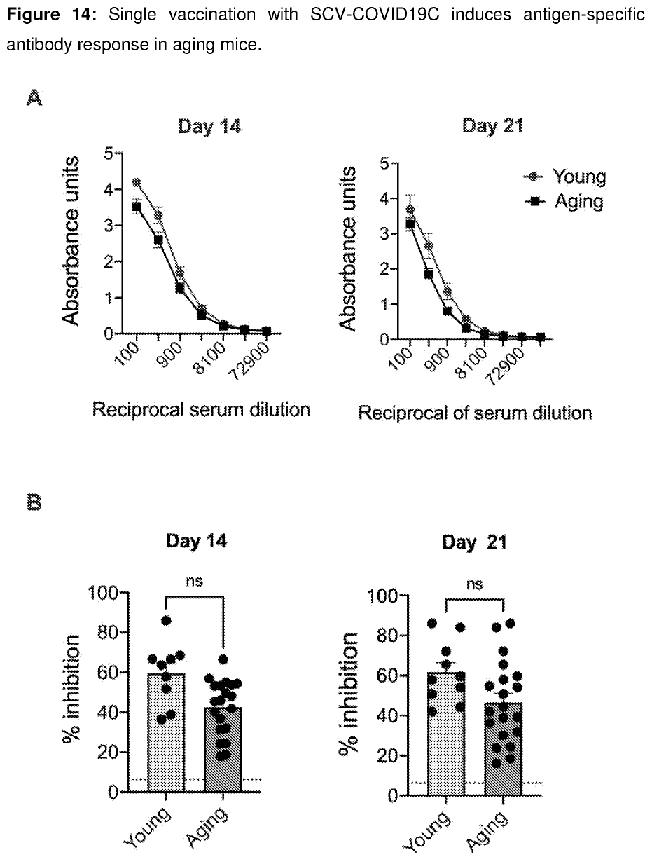

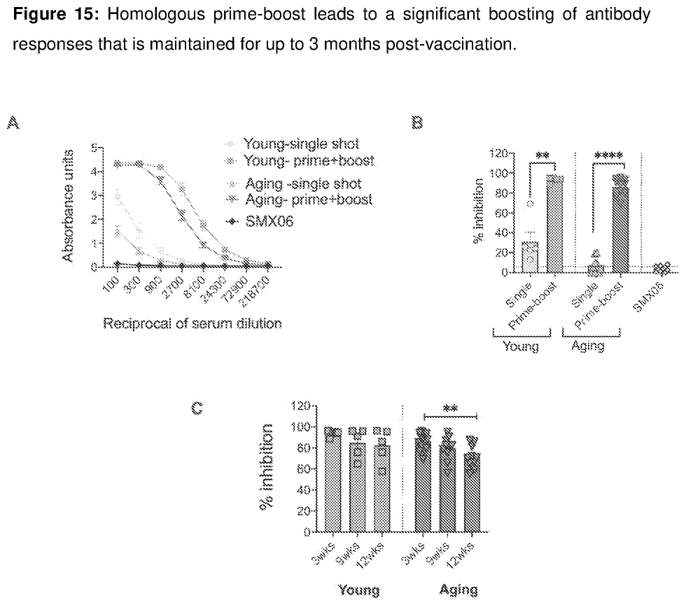

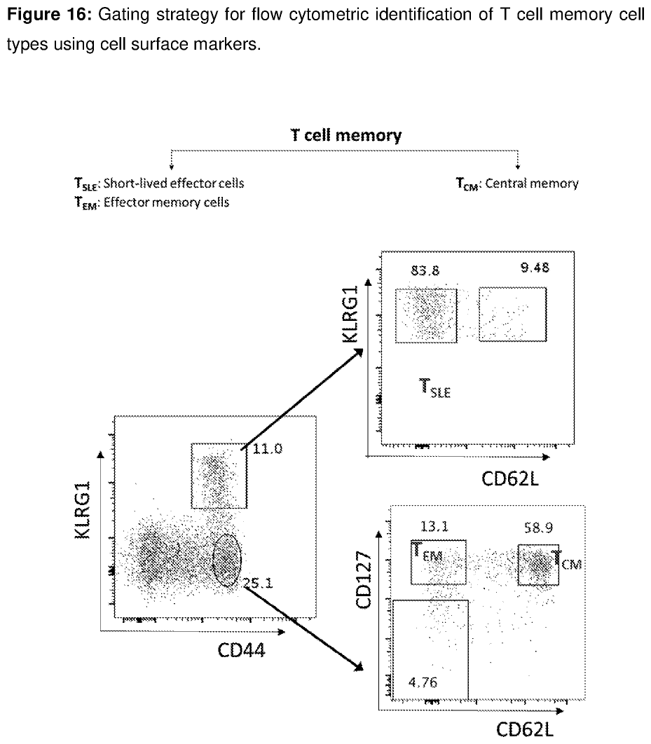

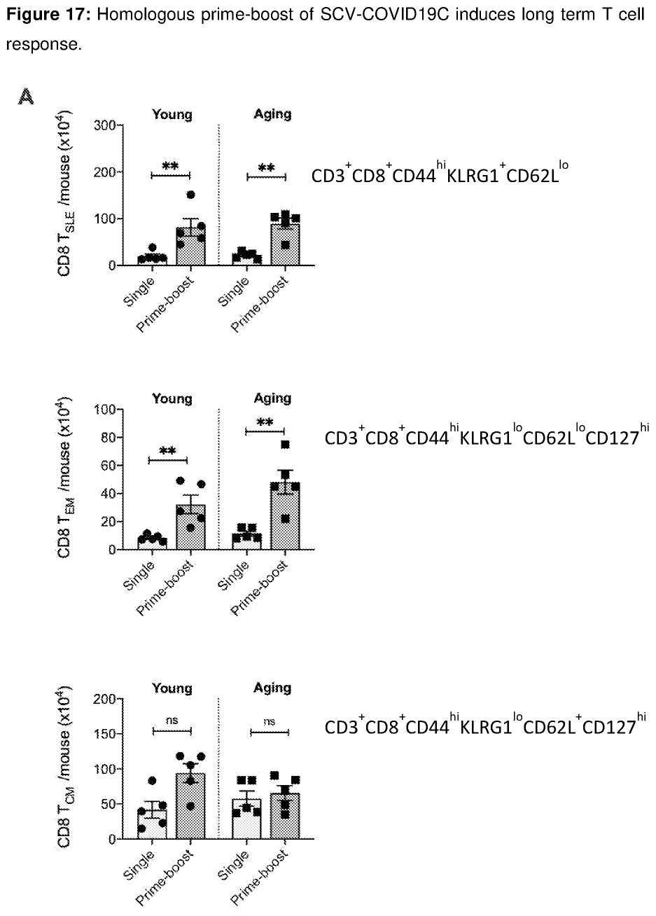

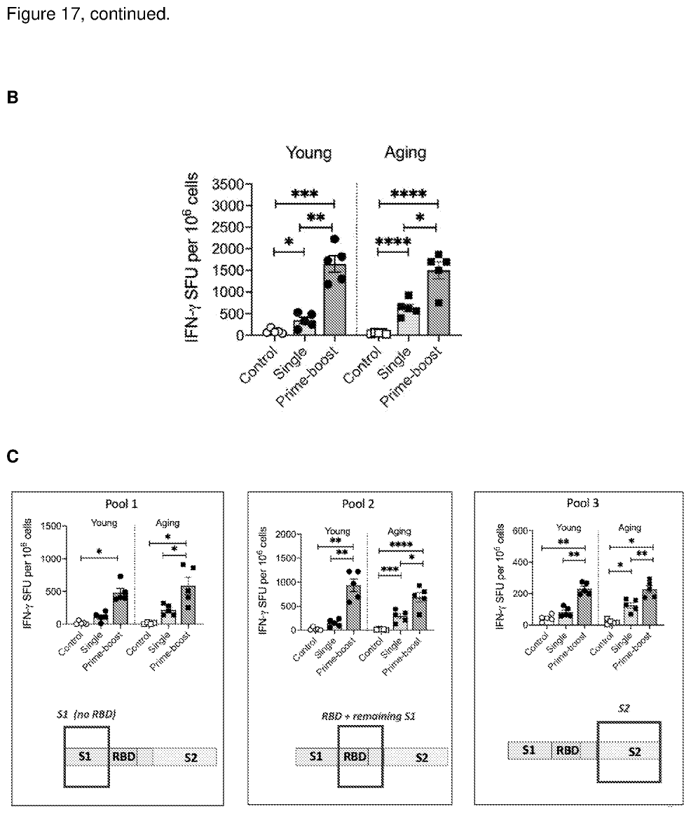

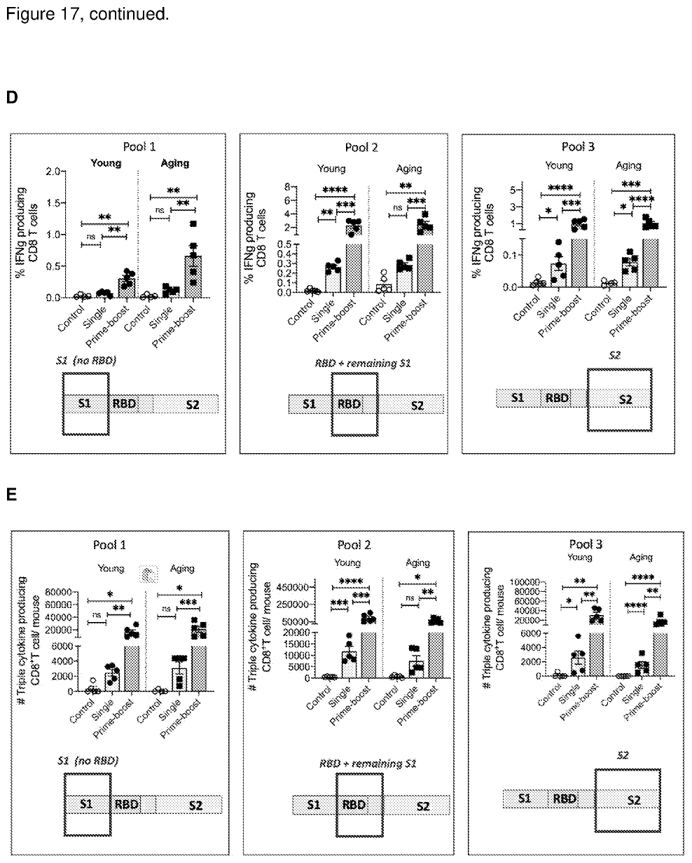

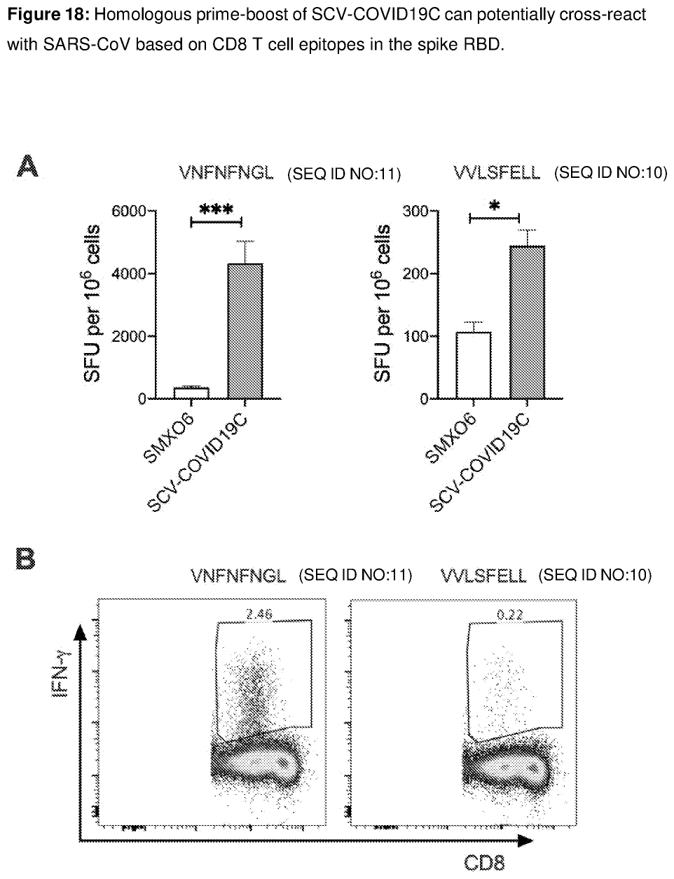

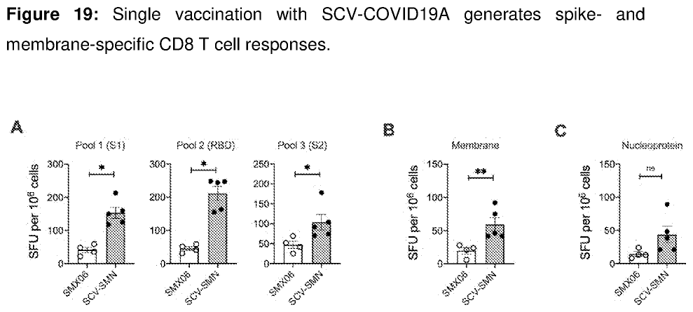

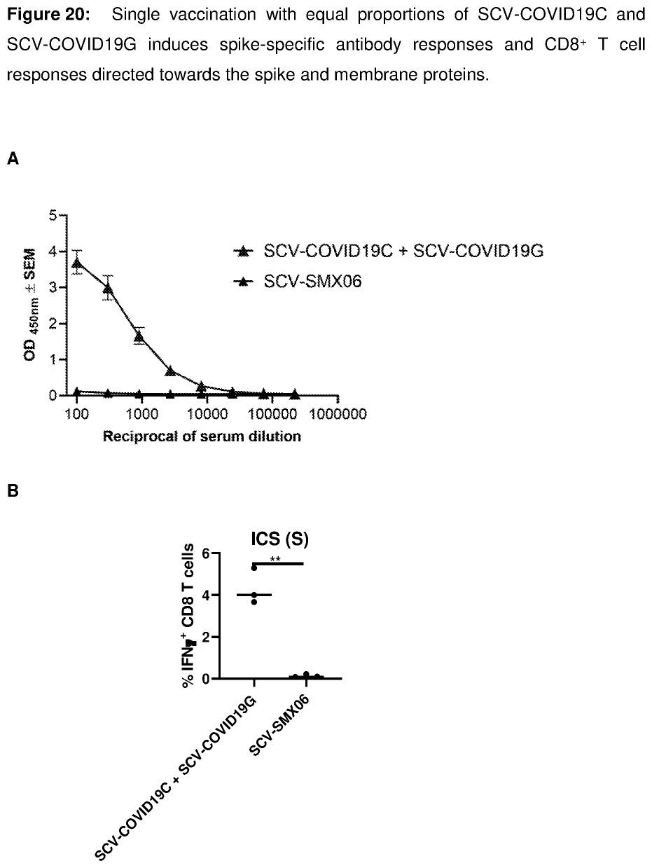

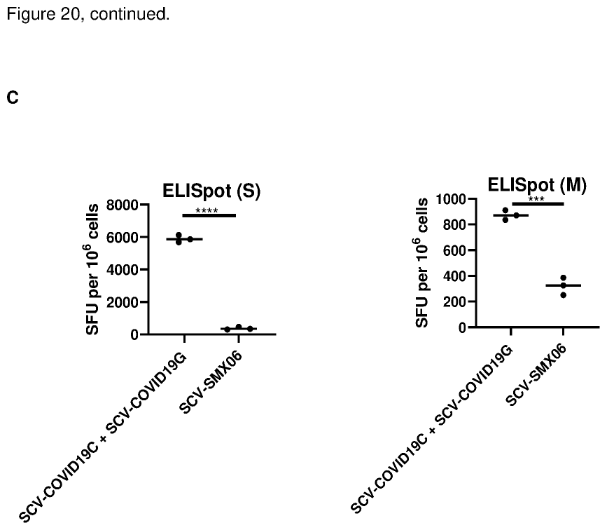

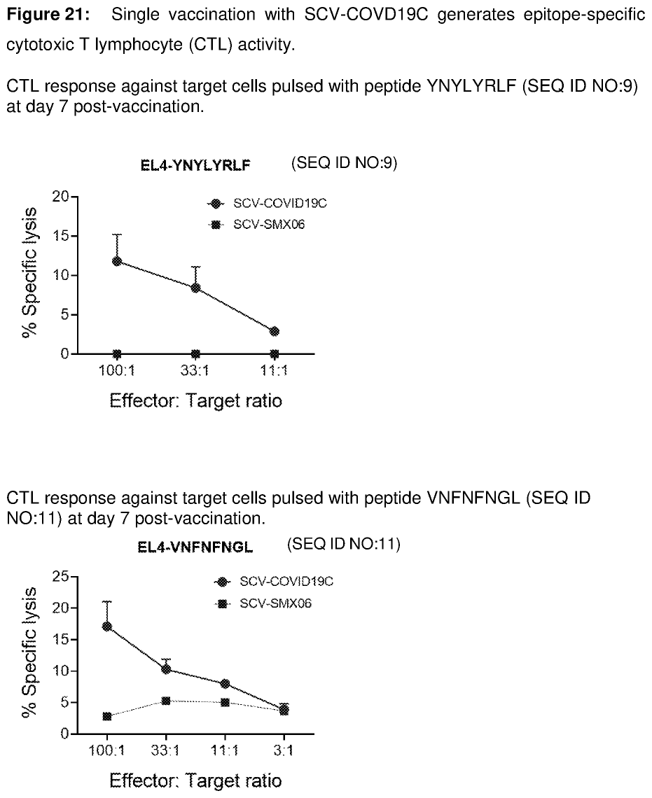

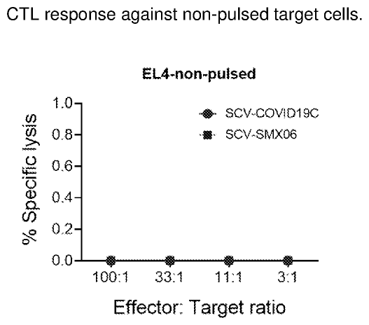

Briefly, to construct recombinant SCV-COVID19 vaccines (FIG. 4), homologous recombination is carried out in either in BC19A-12 cells or in 5D07-1 cells (where CP77 host range selection is required). Cells were infected at a multiplicity of infection (moi) of 0.01 pfu/cell for 1 hour with SCV-SMX06, a replication-incompetent vaccinia virus derived from the Copenhagen strain that has the D13L, A39R, B7/B8R, and C3L ORFs deleted. Infected cells were then transfected with the Not I digested homologous recombination plasmid using a transfection reagent such as EFFECTENE® (Qiagen). The infected/transfected cells are incubated for 2 to 3 days until fluorescent cells could be seen. The recombinant virus was purified from the parent virus using repeated positive drug selection and fluorescence-based single cell sorting. After parent virus removal was achieved (confirmed by PCR), any additional expression cassettes were inserted in iterations of homologous recombination and purification. In the absence of parent virus, selection pressure was removed to allow deletion of selection genes via intramolecular recombination between the 150 bp repeats during infection of BC19A-12 cells. Viruses without selection markers were enriched and purified by fluorescence-based single cell sorting and/or limiting dilution. Once the virus population was selection marker free, candidate clones were then amplified in BC19A-12 cells to generate virus seed stocks that were validated by PCR and DNA sequencing for transgene location and integrity, and western blot or other immunostaining technique for transgene expression.

Summary of Construction Strategies

SCV is derived from the Copenhagen strain of vaccinia virus that has been genetically engineered to delete D13L, a gene encoding an essential viral assembly protein, effectively rendering the SCV virus unable to generate infectious viral progeny.

SCV-SMX06 is a version of SCV with additional gene deletions, specifically that of immune modulatory genes A39R, B7/B8R, and C3L. Genes were deleted sequentially using homologous recombination as illustrated in FIG. 2 where D13L, A39R, B7/B8R, and C3L regions between F1 and F2 recombination arms are deleted. SCV-SMX06 is the base SCV virus used to construct the variations of SCV-COVID vaccines. Insertion of transgenes into the deletion sites, when required, were facilitated by using the same F1 and F2 recombination arms (FIG. 3).

Furthermore, in variations where SARS-CoV-2 spike or an immunogenic part thereof is inserted as transgene, the A41 L ORF is deleted as the transgene is inserted. In variations lacking SARS-CoV-2 spike or an immunogenic part thereof, the A41 L gene remains unmodified.

SCV-COVID19 viruses are constructed to provide single vectored vaccines. The single vectored vaccines may be combined as a mixed vaccine.

Recombinant virus SCV-COVID19A is constructed by substituting the A41 L ORF of SCV-SMX06 with an expression cassette encoding the SARS-CoV-2 spike polypeptide under the transcriptional control of a synthetic early/late promoter (for example, the expression cassette having the nucleotide sequence defined in SEQ ID NO:1), and by inserting an expression cassette comprising the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06.

Recombinant virus SCV-COVID19B is constructed by substituting the A41 L ORF of SCV-SMX06 with an expression cassette encoding the SARS-CoV-2 spike polypeptide under the transcriptional control of a native early/late promoter(for example, the expression cassette having the nucleotide sequence defined in SEQ ID NO:2), and by inserting an expression cassette comprising the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06.

Recombinant virus SCV-COVID19C is constructed by substituting the A41 L ORF of SCV-SMX06 with an expression cassette encoding the SARS-CoV-2 spike polypeptide under the transcriptional control of a synthetic early/late promoter (for example, the expression cassette having the nucleotide sequence defined in SEQ ID NO:1).

Recombinant virus SCV-COVID19D is constructed by substituting the A41 L ORF of SCV-SMX06 with an expression cassette encoding the SARS-CoV-2 spike polypeptide under the transcriptional control of a native early/late promoter (for example, the expression cassette having the nucleotide sequence defined in SEQ ID NO:2).

Recombinant virus SCV-COVID19E is constructed by inserting between the J2R and J3R genes of SCV-SMX06 an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:5).

Recombinant virus SCV-COVID19F is constructed by substituting the A41 L ORF of SCV-SMX06 with an expression cassette encoding the 51 receptor-binding domain subunit of the SARS-CoV-2 spike polypeptide under the transcriptional control of a synthetic early/late promoter (for example, the expression cassette having the nucleotide sequence defined in SEQ ID NO:3).

Recombinant virus SCV-COVID19G is constructed by inserting an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06.

Recombinant virus SCV-COVID19H is constructed by substituting the A41 L ORF of a SCV-SMX06 vaccinia virus strain with an expression cassette encoding the SARS-CoV-2 spike polypeptide under the transcriptional control of a synthetic early/late promoter (for example, the expression cassette having the nucleotide sequence defined in SEQ ID NO:1), and inserting an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06, and by inserting an expression cassette encoding the SARS-CoV-2 envelope polypeptide under the transcriptional control of a synthetic early/late promoter (for example, the expression cassette having the nucleotide sequence defined in SEQ ID NO:8) into the B7/B8R ORF deletion site of SCV-SMX06.

Recombinant virus SCV-COVID191 is constructed by substituting the A41 L ORF of a SCV-SMX06 vaccinia virus strain with an expression cassette encoding the SARS-CoV-2 spike polypeptide under the transcriptional control of a native early/late promoter (for example, the expression cassette having the nucleotide sequence defined in SEQ ID NO:2), and by inserting an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06, and by inserting an expression cassette encoding the SARS-CoV-2 envelope polypeptide under the transcriptional control of a synthetic early/late promoter (for example, the expression cassette having the nucleotide sequence defined in SEQ ID NO:8) into the B7/B8R ORF deletion site of SCV-SMX06.

Recombinant virus SCV-COVID19J is constructed by substituting the A41 L ORF of a SCV-SMX06 vaccinia virus strain with an expression cassette encoding the 51 receptor-binding domain subunit of the SARS-CoV-2 spike polypeptide under the transcriptional control of a synthetic early/late promoter (for example, the expression cassette having the nucleotide sequence defined in SEQ ID NO:3), and by inserting an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06.

Recombinant virus SCV-COVID19K is constructed by substituting the A41 L ORF of a SCV-SMX06 vaccinia virus strain with an expression cassette encoding the 51 receptor-binding domain subunit of the SARS-CoV-2 spike polypeptide under the transcriptional control of a synthetic early/late promoter (for example, the expression cassette having the nucleotide sequence defined in SEQ ID NO:3), and by inserting an expression cassette encoding the SARS-CoV-2 membrane polypeptide under the transcriptional control of a fowlpox early/late promoter and nucleocapsid polypeptide under the transcriptional control of a synthetic early/late promoter (for example, the expression cassette having the nucleotide sequence defined in SEQ ID NO:4) into the D13L ORF deletion site of SCV-SMX06, and by inserting an expression cassette encoding the SARS-CoV-2 envelope polypeptide under the transcriptional control of a synthetic early/late promoter (for example, the expression cassette having the nucleotide sequence defined in SEQ ID NO:8) into the B7/B8R ORF deletion site of SCV-SMX06.

Recombinant virus SCV-COVID19L is constructed by inserting an expression cassette encoding the SARS-CoV-2 membrane polypeptide under the transcriptional control of a fowlpox early/late promoter (for example, the expression cassette having the nucleotide sequence defined in SEQ ID NO:6) into the C3L ORF deletion site of SCV-SMX06.

Recombinant virus SCV-COVID19M is constructed by inserting an expression cassette encoding the SARS-CoV-2 nucleocapsid polypeptide under the transcriptional control of a synthetic early/late promoter (for example, the expression cassette having the nucleotide sequence defined in SEQ ID NO:7) into the D13L ORF deletion site of SCV-SMX06.

SARS-CoV-2 antigen insertion regions within the SCV-COVID19 viral vaccines are illustrated in FIG. 5. Specifically, SARS-CoV-2 spike polypeptide under synthetic early/late promoter in the A41 L ORF (FIG. 5A), SARS-CoV-2 spike polypeptide under native early/late promoter in the A41L ORF (FIG. 5B), SARS-CoV-2 S1 subunit of the spike polypeptide in the A41 L ORF (FIG. 5C), SARS-CoV-2 membrane and nucleocapsid polypeptides in the D13L ORF (FIG. 5D), SARS-CoV-2 membrane and nucleocapsid polypeptides in the intergenic site between J2R and J3R (FIG. 5E), SARS-CoV-2 envelope polypeptide in the B7/B8R ORF (FIG. 5F), SARS-CoV-2 membrane polypeptide in the C3L ORF (FIG. 5G), and SARS-CoV-2 nucleocapsid polypeptide in the D13L ORF (FIG. 5H).

Table 1 summarizes the SCV-COVID19 insertion and deletion sites within the SCV-SMX06 genome. For SCV-COVID19 species comprising a spike or S1 transgene, the A41 L gene is deleted. J203R indicates an intergenic insertion site where antigens are inserted without modification to adjacent J2R and J3R genes. Unmodified sites are indicated by ‘+’ whereas ‘−’ indicates the ORF of the gene has been deleted.

TABLE 1
Summary of example SCV-COVID19 insertion and deletion sites.

| | A41L | D13L | B7/B8R | C3L | A39R | J2↓J3R |
|---|---|---|---|---|---|---|
| SCV- | S (prPs) | M and N | − | − | − | + |
| COVID19A | | | | | | |
| SCV- | S (pr7.5) | M and N | − | − | − | + |
| COVID19B | | | | | | |
| SCV- | S (prPs) | − | − | − | − | + |
| COVID19C | | | | | | |
| SCV- | S (pr7.5) | − | − | − | − | + |
| COVID19D | | | | | | |
| SCV- | + | − | − | − | − | M and |
| COVID19E | | | | | | N |
| SCV- | S1 | − | − | − | − | + |
| COVID19F | | | | | | |
| SCV- | + | M and N | − | − | − | + |
| COVID19G | | | | | | |
| SCV- | S (prPs) | M and N | E | − | − | + |
| COVID19H | | | | | | |
| SCV- | S (pr7.5) | M and N | E | − | − | + |
| COVID19I | | | | | | |
| SCV- | S1 | M and N | − | − | − | + |
| COVID19J | | | | | | |
| SCV- | S1 | M and N | E | − | − | + |
| COVID19K | | | | | | |
| SCV- | + | − | − | M | − | + |
| COVID19L | | | | | | |
| SCV- | + | N | − | − | − | + |
| COVID19M | | | | | | |

Furthermore, a combination vaccine is prepared by mixing equal proportions of two single-vectored vaccines, specifically that of SCV-COVID19C and SCV-COVID19G, and delivered via a single syringe.

Furthermore, a combination vaccine is prepared by mixing equal proportions of two single-vectored vaccines, specifically that of SCV-COVID19D and SCV-COVID19G, and delivered via a single syringe.

Furthermore, a combination vaccine is prepared by mixing equal proportions of two single-vectored vaccines, specifically that of SCV-COVID19C and SCV-COVID19E, and delivered via a single syringe.

Furthermore, a combination vaccine is prepared by mixing equal proportions of two single-vectored vaccines, specifically that of SCV-COVID19D and SCV-COVID19E, and delivered via a single syringe.

### Example 2

Single Vaccination with SCV-COVID19D Generates Neutralizing SARS-CoV-2 Antibodies and a Th1-Biased Antibody Profile in Outbred and Inbred Mice

### Experimental Strategy

Groups of 6-9 week old male C57BL/6 mice or ARC Swiss mice (n=5 mice per group) were vaccinated via intramuscular administration with SCV-COVID19D (10⁷, 10⁸ PFU) or the vector control SCV-SMX06. Blood samples were obtained before vaccination and 21 days thereafter. Levels of S1-specific IgG levels and SARS-CoV-2 virus-specific neutralizing antibody levels were determined by end-point ELISA and neutralization assay.

The two cohorts of mice were used to account for genetic heterozygosity. The first cohort used the inbred mouse strain C57BL/6 to minimize phenotypic or trait variability and thus improving reproducibility while the second group utilized the outbred mouse strain Swiss to represent genetic diversity and therefore more generalizability of responses across population.

### Virus Neutralization Test

Sera were heat-inactivated for 30 minutes at 56° C. and stored at −80° C. until day of processing. 96-well plates containing Vero cells were also cultured to ensure monolayer confluence on day of processing. On the day of the neutralization assay, two-fold serial dilutions of serum were prepared in Minimum Essential Medium (MEM) culture medium and Vero plates were washed with infection medium composed of MEM and antibiotics and trypsin. 100 TCID₅₀ per μl of SARS-CoV-2 was added to each dilution of the pre-prepared sera dilutions and incubated at room temperature for 1 hour, with occasional rocking. The virus:serum mixture was then added to the Vero cells and incubated at 37° C. and 5% CO₂ and then microscopically monitored and scored for cytopathic effects 4 days post-procedure. The virus neutralization titre was expressed as the reciprocal value of the highest dilution of the serum which still inhibited virus replication.

### Enzyme-Linked Immunosorbent Assay for Mouse Sera

MaxiSorp plates (Nunc) were coated with S1 (120 ng/well) in PBS for overnight adsorption at 4° C. Plates were washed in PBS/Tween (0.05% v/v) and wells blocked using 3% skim milk in PBS/Tween for 1 hr at room temperature. Serially diluted mouse serum samples were added and incubated room temperature for 2 hours. Plates were washed and Horseradish peroxidase-conjugated goat anti-mouse IgG was added to all wells for 1 hr at room temperature. After washing TMB liquid substrate (Sigma) was added and reaction stopped using 3M HCl. Optical density (OD) values for each well were measured at 450 nm. Endpoint titres were calculated as follows: the login OD against login sample dilution was plotted and a regression analysis of the linear part of this curve allowed calculation of the endpoint titre. The endpoint titres were calculated when the OD readings reached the mean absorbance values of the negative serum samples plus three times the standard deviation. The results of the IgG subclass ELISA are presented using OD values.

### Results

Virus-specific neutralizing antibodies were detected in all mice vaccinated with SCV-COVID19D (FIG. 6A). At 10⁷ PFU dose of vaccination, the titres of neutralizing antibodies generated by outbred Swiss mice and inbred C57131/6 mice were higher than the SCV-SMX06, with the levels of neutralizing antibodies comparable between the outbred and inbred mice at the same dose. At 8 PFU dose of vaccination for Swiss mice, there was an increase in the neutralizing antibody titre. Total IgG titres were also detected against the S1 subunit of the spike protein in both outbred Swiss strain and C57BL/6 mice (FIG. 6B). Profiling of the IgG subclasses by ELISA showed higher levels of S1-specific IgG2c compared to IgG1, indicating a predominantly Th1 response post vaccination (FIG. 6C).

### Example 3

Single Vaccination with SCV-COVID19D Generates Spike-Specific CD8 T Cell Responses

T cells are critical to generate early control and clearance of many viral infections of the respiratory system. Recent studies in transgenic mouse models provided evidence that T cells are utilized in viral clearance and disease resolution following SARS-CoV-2 infection. Herein, we define whether immunization with SCV-COVID19D elicits an early T cell response that would be potentially beneficial in dampening disease severity.

### Experimental Strategy

Groups of 6-9 week old male C57BL/6 mice (n=5 mice per group) were vaccinated via intramuscular administration with SCV-COVID19D or the vector control SCV-SMX06 at a dose of 10⁷ PFU. Spike-specific T cell responses (using peptides spanning the full length of the spike protein) were detected by ELISpot and intracellular cytokine staining (ICS) 3 months post-vaccination.

### ELISpot Assay

Dilutions of single cell suspension of murine splenocytes were prepared by passing cells through 70 μM cell strainers and ACK lysis prior to resuspension in complete media. For analysis of interferon-gamma (IFNγ) production by ELISpot, a PVDF ELISpot plate (MabTech) was incubated with the anti-mouse IFNγ coating antibody overnight then blocked with the cell culture medium. Cell dilutions were incubated with pools of peptides spanning the entire spike protein (2 μg/ml per peptide) in the ELISpot plate 18 to 20 hours in a 37° C. humidified incubator with 5% CO₂. After stimulation, IFNγ spot forming units (SFU) were detected by staining membranes with anti-mouse IFNγ biotin detection antibody followed by streptavidin-Alkaline Phosphatase and colour development with BCIP/NBT substrate kit (MabTech). Spots representing cytokine-secreting T cells were quantified using an ELISpot reader.

### Intracellular Cytokine Staining Analysis

To complement and verify the ELISpot results, intracellular cytokine production of IFNγ was performed. Cells were stimulated at 37° C. for 6 hours with pools of peptides spanning the entire spike protein (2 μg/ml per peptide) along with protein transport inhibitor Brefeldin A. Cells were stained for surface markers CD3 and CD8, then fixed with 4% paraformaldehyde. Cells were permeabilized using BD cytofix/perm buffer and intracellular staining for IFNγ was performed. Sample acquisition was performed on a FACS Aria 2 (BD) and data analyzed in FlowJo V10 (TreeStar). Cytokine-secreting T cells were identified by gating on doublet negative live lymphocytes, size, CD3+, CD8+ cells and IFNγ cytokine positive.

### Results

A significant increase in spike-specific IFNγ⁺ producing T cell responses was detected in SCV-COVID19D vaccinated mice compared to the vector control both by ICS (FIG. 7A) and ELISpot (FIG. 7B). The vector control group had low (<100 SFU) or minimal detectable response (FIG. 15B).

### Conclusion

Examples 2 and 3 herein indicate that immunization of animal model with SCV-COVID19 vaccine encoding SARS-CoV-2 spike protein induces cellular and humoral responses as shown by the production of S1-specific antibodies, neutralizing antibodies, and increase in spike-specific IFNγ-secreting CD8+ T cells. These suggest that SCV-COVID19 vaccine may provide prophylactic protection against SARS-CoV-2, the infectious agent for COVID-19.

### Example 4

SCV-COVD19C Elicits Better Spike-Specific Antibody than SCV-COVID19D and SCV-COVID19F

SCV-COVID19C and SCV-COVID19D vaccines differ in the type of poxviral promoter used for expression of antigens. SCV-COVID19C and SCV-COVID19F vaccines differ in the length of transgene inserted, wherein SCV-COVID19C comprises the whole length of the spike protein while SCV-COVID19F comprises only the S1 subunit of the spike protein. Herein, we compared the efficiency of antigen expression of the three vaccines by western blot and evaluated their capacity to induce spike S1-specific antibody response using ELISA.

### Experimental Strategy

Cell lines were infected with SCV-COVID19C, SCV-COVID19D, and SCV-COVID19F and the expression of the spike protein, or S1 subunit, was examined by western blot. Groups of 6-9 week old male C57BL/6 mice (n=5 mice per group) were vaccinated via intramuscular administration with SCV-COVID19C, SCV-COVID19D, SCV-COVID19F or the vector control SCV-SMX06 at a dose of 7 pfu per mouse and spike S1-specific antibody responses were evaluated by ELISA.

### Western Blot

SCV-COVID19C, SCV-COVID19D, and SCV-COVID19F concentrates were lysed in a loading buffer and an equivalent of 8 ug of total protein for each sample were separated on 10% SDS-polyacrylamide gel and transferred onto a nitrocellulose filter membrane. The membranes were blocked and incubated with rabbit mAbs for SARS-CoV-2 Spike RBD (Sino Biological 40592-T62) diluted 1:1000. Bound antibodies were detected using horse radish peroxidase (HRP)-conjugated anti-rabbit IgG followed by enhanced chemiluminescence using Clarity ECL and TMB for membranes (Sigma).

### Enzyme-Linked Immunosorbent Assay for S1-Specific Antibody Endpoint Titres

MaxiSorp plates (Nunc) were coated with S1 (120 ng/well) in PBS for overnight adsorption at 4° C. Plates were washed in PBS/Tween (0.05% v/v) and wells blocked using 3% skim milk in PBS/Tween for 1 hr at room temperature. Serially diluted mouse serum samples were added and incubated at room temperature for 2 hours. Plates were washed and Horseradish peroxidase-conjugated goat anti-mouse IgG was added to all wells for 1 hr at room temperature. After washing TMB liquid substrate (Sigma) was added and reaction stopped using 3M HCl. Optical density (OD) values for each well were measured at 450 nm. Endpoint titres were calculated as follows: the log₁₀ OD against log₁₀ sample dilution was plotted and a regression analysis of the linear part of this curve allowed calculation of the endpoint titre. The endpoint titres were calculated when the OD readings reached the mean absorbance values of the negative serum samples plus three times the standard deviation.

### Results

FIG. 8A shows the expression of SARS-CoV-2 spike protein in cells lysates by Western blot. Lysates for both SCV-COVID19C and SCV-COVID19D showed bands at 180 kDa, reflecting the expression of the full-length spike protein while the lysate for SCV-COVID19F showed a band at 80 kDa reflecting the expression of the S1 subunit of the spike protein. However, SCV-COVID19C exhibited a stronger signal compared to SCV-COVID19D indicating more efficient expression of the spike protein under the synthetic early/late promoter. Comparison of S1-specific antibody responses by ELISA at 21 days post-immunization confirmed higher antibody titres induced by SCV-COVID19C than SCV-COVID19D or SCV-COVID19F (FIG. 8B).

### Conclusion

Example 4 demonstrates that the SCV-COVID19C vaccine which has the spike protein under the control of the synthetic early/late promoter expresses higher levels of spike protein compared to SCV-COVID19D vaccine. Antibody titres demonstrate a higher magnitude of immunogenicity for SCV-COVID19C compared to SCV-COVID19D or SCV-COVID19F. The following examples further investigated the effectiveness of SCV-COVID19C vaccine.

### Example 5

Single Vaccination with SCV-COVID19C Induces Antibody Responses in Inbred and Outbred Mice

### Experimental Strategy

Groups of 6-9 week old female inbred C57BL/6 mice and ARC(s) mice (n=5 mice per group) were vaccinated via intramuscular administration with SCV-COVID19C or the vector-only control SMX06 at a dose of 10⁷ PFU per mouse. Blood samples were obtained before vaccination and on days 14 post-vaccination. Levels of spike (S1)-protein specific IgG levels and SARS-CoV-2 virus-specific neutralizing antibody levels were determined by end-point ELISA and pseudo-neutralization assay (cPass™; Genscript).

The two cohorts of mice were used to account for variations in genetic heterozygosity. The first cohort used the inbred mouse strain C57BL/6 to minimize phenotypic or trait variability and thus improving reproducibility while the second cohort utilized the outbred mouse strain Swiss to represent genetic diversity and therefore more generalizability of responses across population.

### Enzyme-Linked Immunosorbent Assay for S1-Specific Antibody Endpoint Titres

MaxiSorp plates (Nunc) were coated with S1 (120 ng/well) in PBS for overnight adsorption at 4° C. Plates were washed in PBS/Tween (0.05% v/v) and wells blocked using 3% skim milk in PBS/Tween for 1 hr at room temperature. Serially diluted mouse serum samples were added and incubated at room temperature for 2 hours. Plates were washed and Horseradish peroxidase-conjugated goat anti-mouse IgG was added to all wells for 1 hr at room temperature. After washing TMB liquid substrate (Sigma) was added and reaction stopped using 3M HCl. Optical density (OD) values for each well were measured at 450 nm. Endpoint titres were calculated as follows: the log₁₀ OD against log₁₀ sample dilution was plotted and a regression analysis of the linear part of this curve allowed calculation of the endpoint titre. The endpoint titres were calculated when the OD readings reached the mean absorbance values of the negative serum samples plus three times the standard deviation.

cPass™ SARS-CoV-2 Neutralization Antibody Detection

The cPass™ SARS-CoV-2 neutralization antibody detection kit (GenScript, USA) is a blocking ELISA intended for qualitative direct detection of total neutralizing antibodies to SARS-CoV-2 in serum and plasma. Infection with SARS-CoV-2 initiates an immune response which includes the production of antibodies, or binding antibodies, in the blood. Using purified receptor binding domain (RBD), protein from the viral spike (S) protein and the host cell receptor ACE2, the test mimics the virus-host interaction by direct protein-protein interaction in a test tube or a well of an ELISA plate. The highly specific interaction can then be neutralized, the same manner as in a conventional Virus Neutralization Test. Briefly, samples and controls diluted with sample dilution buffer and pre-incubated with the HRD-conjugated RBD to allow the binding of the circulating neutralization antibodies to HRP-RBD. The mixture is then added to the capture plate, which is pre-coated with ACE2 protein. The unbound HRP-RBD as well as and HRP-RBD bound to non-neutralizing antibody will be captured on the plate, while the circulating neutralization antibodies HRP-RBD complexes remain in the supernatant and are removed during washing. Following a wash cycle, TMB substrate solution is added followed by the Stop Solution and the reaction is then quenched and the color turns yellow. The absorbance of the final solution is read at 450 nm in a microplate reader and the percent signal inhibition is determined using the following formula:

Percent Signal Inhibition=(1−OD value of sample/OD value of negative control)×100%

Percent signal inhibition refers to the qualitative detection of SARS-CoV-2 total neutralizing antibodies. The cPass™ kit has been authorized by the Food and Drug Administration (FDA) for use in evaluation of vaccine efficacy and assessments of herd immunity.

### Results

FIG. 9A shows S1-specific IgG levels observed in the two cohorts of mice vaccinated with SCV-COVID19C compared to the vector-only control SMX06 on day 14 post-vaccination. S1-specific IgG was detected in both inbred and outbred mice (FIG. 9A), with outbred mice generating higher levels of antibodies compared to inbred mice (FIG. 9B). Consistent with this, higher levels of neutralizing antibody were detected by the cPass assay in outbred ARC(s) mice compared to inbred C57BL/6 mice (FIG. 9C), with both cohorts generating higher neutralizing antibodies compared to the vector control SMX06. These results demonstrate that the SCV-COVID19C can induce spike-specific antibodies with neutralizing capacity in both outbred mice (representative of genetic heterozygosity and therefore translation to the human population) and inbred mice. Based on these results and reagent availability for the C57BL/6, the inbred mice was chosen as an experimental model for further investigation of vaccine-mediated immune responses.

### Example 6

Single Vaccination with SCV-COVID19C Induces Robust Spike-Specific T Cell Response

T cells are critical to generate early control and clearance of many viral infections of the respiratory system. Recent studies in transgenic mouse models provided evidence that T cells are utilized in viral clearance and disease resolution following SARS-CoV-2 infection. Herein, we define whether immunization with SCV-COVID19C elicits an early T cell response that would be potentially beneficial in dampening disease severity.

### Experimental Strategy

Male (n=3 per group) and female (n=3 per group) 6-9 week old C57BL/6 mice were grouped into three: (1) naïve mice/no vaccine, (2) mice vaccinated with vector-only control SMX06, and (3) mice vaccinated with a single dose of SCV-COVID19C (10⁷ PFU).

### Method

On day 7 post-vaccination, spleens were collected and processed for single cell characterization via flow cytometry. Single cell preparations from the spleen were isolated by standard methods. Briefly, using the plunger of a small syringe, spleens were crushed through a 70 μm cell strainer. The resultant single cell suspension was spun (300×g, 5 min), and resuspended in 1 mL of ammonium-chloride-potassium (ACK) lysis buffer for 5 min to eliminate red blood cells. RPMI culture medium supplemented with 10% fetal bovine serum (FBS) was then added to neutralize the lysis buffer. Splenocytes were washed twice with PBS and resuspended at 2×10⁷ cells/mL in preparation for multi-parameter flow cytometry.

CD8 and CD4 T cell responses were evaluated using intracellular cytokine staining as described in Example 3. Intracellular cytokine staining was used to assess the production of cytokines IFN-γ, TNF-α, and IL-2 using the gating strategy shown in FIG. 10. Production of granzyme B, a key cytotoxic effector molecule produced by effector CD8 T cells, was also measured via flow cytometry.

Two peptide pools comprised of overlapping peptides spanning the S1 and S2 regions of the SARS-CoV-2 spike protein were used for measuring the specificity of vaccine-mediated CD8 T and CD4 T cell responses. Spike pool 1 was comprised of 15 AA length peptides with overlapping 11mers spanning the whole sequence of S1 and spike pool 2 was comprised of 15 AA length peptides with overlapping 11mers spanning the whole sequence of S2. In addition, a peptide mix (pepmix) comprising of overlapping peptide pools containing immunodominant T cell epitopes YNYLYRLF (SEQ ID NO:9), VVLSFELL (SEQ ID NO:10), and VNFNFNGL (SEQ ID NO:11) within the RBD region of S1. These T cell epitopes were previously identified in mouse studies of CD8 T cell activation in SARS-CoV. Cells were stimulated at 37° C. for 6 hours with 2 μg/ml/per peptide per condition along with protein transport inhibitor Brefeldin A and the cytokines produced were analyzed by ICS.

### Results: Spike-Specific Triple-Cytokine-Positive CD8 T Cell Response

Intracellular cytokine staining on day 7 post-vaccination showed that a single dose of SCV-COVID19C (FIG. 11A; bottom panel) lead to significant increase in the number of spike-specific IFN-γ CD8 T cells whereas CD8 T cells from naïve (FIG. 11A; top panel) and vector-only (FIG. 11A; middle panel) control mice produce minimal levels of IFN-γ. Spike-specific IFN-γ-producing T cells induced post vaccination with SCV-COVID19C were poly-functional and secreted additional cytokines, with more than half are TNF-α-producing as well. About 15% of cells are also classified as triple-cytokine-producers (IFN-γ, TNF-α, and IL-2) and these cells are considered as a hallmark of good-quality T cell response (FIG. 11A; bottom panel).

FIG. 11B is a graphical representation of the number of single, double, and triple-cytokine-producing IFN-γ CD8 T cells that are spike pool 1 (S1)-specific (left panel), and spike pool 2 (S2)-specific (middle panel) and epitope-specific (YNYLYRLF (SEQ ID NO:9), VVLSFELL (SEQ ID NO:10), and VNFNFNGL (SEQ ID NO:11); right panel) in all groups of experimental mice.

### Results: Spike-Specific Granzyme-B-Producing CD8 T Cell Response

Compared to naïve mice, SCV-COVID19C generated granzyme-B-producing CD8 T cells post-vaccination (FIG. 11C).

### Results: Spike-Specific IFN-γ-Producing CD4 T Cell Response

To assess whether a single vaccination of SCV-COVID19C induced antigen-specific CD4 T cell response, splenocytes were restimulated with peptide pools spanning the S1 (pool 1) and S2 (pool 2) regions of SARS-CoV-2. Vaccination with SCV-COVID19C induces S1- and S2-specific triple-cytokine-producing CD4 T cells and this is represented in FIG. 11D.

### Conclusion

A single vaccination of SCV-COVID19C generates CD8 T cells with cytotoxic potential (granzyme-B-producing) and spike-specific polyfunctional CD8 and CD4 T cells.

### Example 7

Pre-Existing Immunity does not Affect the Quantity and Quality of Spike-Specific Antibody Responses Following Administration of a Single-Dose of SCV-COVID19C Vaccine

The influence of pre-existing immunity to viral vectors is a major issue for the development of viral vectored vaccines. There is a potential drawback for the use of orthopoxvirus-based vaccines in that a proportion of the adult human population has immunity against the vaccine vector due to smallpox vaccination campaigns that were conducted until the mid 1970s and ultimately led to the eradication of smallpox. Thus, there is considerable concern for interference of orthopoxvirus-specific pre-existing immunity with subsequent SCV-COVID19 vaccinations, that may result in reduced vaccine immunogenicity and efficacy. Herein, we investigated the effect of pre-existing vaccinia virus immunity on the immunogenicity of either a single shot of SCV-COVID19C or homologous prime-boost of the same vaccine in the pre-clinical mouse model.

### Experimental Strategy

A mix of female and male 6-9 week old C57BL/6 mice (n=5 mice per group) were divided into two treatment groups: (1) mice administered with vaccinia virus 40 days prior to vaccination with SCV-COVID19C at a dose of 10⁷ PFU/mouse to induce a condition of pre-existing immunity against poxvirus, and (2) naïve mice with no pre-existing immunity vaccinated with SCV-COVID19C at a dose of 10⁷ PFU/mouse.

Magnitude and longevity of antigen-specific antibody response were monitored using blood samples obtained on days 28, 44, and 80 post-vaccination. Levels of spike (S1)-protein specific IgG levels and SARS-CoV-2 virus-specific neutralizing antibody levels were determined by end-point ELISA and pseudo-neutralization assay (cPass™; Genscript).

### Method

Enzyme-linked immunosorbent assay for S1-specific antibody endpoint titres and cPass™ SARS-CoV-2 neutralization antibody detection as described in Example 5.

### Results

A single shot of SCV-COVID19C induced comparable levels of S1-specific antibodies for mice with and without pre-existing immunity on 28, 44, and days post-vaccination (FIG. 12A). Consistent with this data, the levels of neutralizing antibodies (assayed using the cPass™ kit from Genscript) was also comparable between the two groups of mice (FIG. 12B).

Pre-Existing Immunity does not Affect Antibody Responses after Prime-Boost Vaccination

### Experimental Strategy

A mix of female and male 6-9 week old C57BL/6 mice were divided into two treatment groups: (1) mice administered with vaccinia virus 40 days prior to vaccination with SCV-COVID19C in a homologous prime-boost strategy (on days 0 and 28) to induce a condition of pre-existing immunity against poxvirus, and (2) naïve mice with no pre-existing immunity vaccinated with SCV-COVID19C in a homologous prime-boost strategy (on days 0 and 28).

Blood samples were obtained on day 28 (pre-boost) and days 14 and 50 post-booster dose to monitor the magnitude and longevity of antibody response. Levels of spike (S1)-protein specific IgG levels and SARS-CoV-2 virus-specific neutralizing antibody levels were determined by end-point ELISA and pseudo-neutralization assay (cPass™ Genscript).

### Method

Enzyme-linked immunosorbent assay for S1-specific antibody endpoint titres and cPass™ SARS-CoV-2 neutralization antibody detection as described in Example 5.

### Results

A significant increase in the spike (S1)-specific antibody response was observed following the administration of a booster dose at D28. Pre-existing immunity did not impact the S1-specific antibody levels (FIG. 13A) and neutralizing antibody levels (FIG. 13B).

### Conclusion

Pre-existing immunity does not impact the quality, quantity or kinetics of antigen-specific antibody responses following vaccination with SCV-COVID19C either in a single dose or a homologous prime-boost strategy.

### Example 8

Single Vaccination with SCV-COVID19C Induces Antigen-Specific Antibody Response in Aging Mice

Age is one of the most significant risk factors for poor health outcomes after SARS-CoV-2 infection, therefore it is desirable that any new vaccine candidates should elicit a robust immune response in older adults. Herein, we tested the antibody response induced by a single dose of SCV-COVID19C vaccination in aging mice.

### Experimental Strategy

To evaluate the level of antibody response according to age, groups of 6-9 week old females (n=10; referred to as young mice) and 9-10 month old females (n=20; referred to as aging mice) C57BL/6 mice were vaccinated via intramuscular administration with SCV-COVID19C (10⁷ PFU/mouse). Blood samples were collected on days 14 and 21 post-vaccination and serum isolated for antibody analysis. Levels of spike (S1)-protein specific IgG levels and SARS-CoV-2 virus-specific neutralizing antibody levels were determined by end-point ELISA and pseudo-neutralization assay (cPass™ Genscript).

### Method

Enzyme-linked immunosorbent assay for S1-specific antibody endpoint titres and cPass™ SARS-CoV-2 neutralization antibody detection as described in Example 5.

### Results

Levels of S1-specific antibodies were comparable between young and aging mice at day 14 and day 21 post-vaccination (FIG. 14A). Similarly, no differences in the neutralizing antibody titres were detected between young and aging mice at day 14 and day 21 post-vaccination (FIG. 14B).

### Conclusion

A single dose of SCV-COVID19C induces antigen-specific immune responses in aging mice comparable to the levels seen in young mice.

### Example 9

Homologous Prime-Boost Leads to a Significant Boosting of Antibody Responses that is Maintained for Up to 3 Months Post-Vaccination

To test whether a prime-boost strategy can enhance the humoral response in young and aging mice, a homologous prime-boost approach was evaluated.

### Experimental Strategy

Twenty-eight (28) days after receiving a first shot (prime dose) of the SCV-COVID19C (10⁷ PFU) vaccine, a boost of SCV-COVID19C (10⁷ PFU) was administered via intramuscular injection in young mice and aging mice. At 21 days post-boost, blood samples were obtained for the assessment of S1-specific antibody levels and neutralizing antibody titres. For longevity studies, blood samples were obtained for the assessment of S1-specific antibody titre and percent inhibition of neutralizing antibodies at 3 weeks, 9 weeks, and 12 weeks post-boost.

### Method

Enzyme-linked immunosorbent assay for S1-specific antibody endpoint titres and cPass™ SARS-CoV-2 neutralization antibody detection as described in Example 5.

### Results

Administration of a booster dose of the SCV-COVID19C in a homologous prime-boost vaccination regimen significantly increases spike (S1)-specific antibody and neutralizing antibody responses in both young and aging mice at 21 days post-boost (FIG. 15A,B). No statistical difference was noted in the neutralizing antibody levels between young and aging mice at day 21 post-boost. Neutralizing antibody levels were analyzed at weeks 3, 9, and 12 post-boost and it was observed that the antibody levels were maintained with no significant difference noted in time-matched comparison of young and aging mice (FIG. 15C).

### Conclusion

The quantity and quality of spike-specific antibody responses were significantly boosted by the administration of a second dose of the SCV-COVID19C vaccine, with antibody responses maintained up to 3 months post-boost (at the time of analysis).

### Example 10

### Homologous Prime-Boost of SCV-COVID19C Induces Long Term T Cell Response

The presence of memory CD8 T cells is particularly important in responding to viral infections as it complements the humoral response by promoting efficient pathogen clearance. T cell memory acts as a formidable secondary defense if the protective ability or magnitude of neutralizing antibodies is compromised. Most importantly, long term T cell response may indicate that immunity conferred by the vaccination may be long-lasting.

### Experimental Strategy

Treatment groups are divided into two cohorts: 6-9 week old young mice and 9-10 month old aging mice. Within each cohort, mice were immunized with either a single shot of SCV-COVID19C (10⁷ PFU), a homologous prime-boost with the second shot administered on day 28 post-prime (10⁷ PFU, 10⁷ PFU), or the vector-only control SMX06.

### Method

Multi-color flow cytometry was used to characterize the memory T cell population as short-lived effector cells (T_{SLE}; identified by high expression of CD44, KLRG1, and low expression of CD62L), effector memory cells (T_{EM}; identified by high expression of CD44, and low expressions of KLRG1 and CD62L) and central memory cells (T_{CM}, identified by high expression of CD44 and CD62L and low expression of KLRG1) (FIG. 16).

ELISpot assay (described previously) was used to quantify IFN-γ-producing T cells while intracellular cytokine staining (described previously) was used to identify cells capable of producing cytokines such as IFN-γ, TNF-α, and IL-2.

To evaluate the nature of the IFN-γ T cell responses elicited by SCV-COVID19C vaccination, we restimulated the CD8 T cells with three different peptide pools spanning the sequence of the following regions: the S1 sans the RBD, RBD and remaining S1 region after RBD, and the S2.

### Results: Homologous Prime-Boost Expands the CD8 Effector T Cell Population

Total number of effector memory and central memory CD8 T cells in young and aging mice vaccinated with either single dose or prime-boost of SCV-COVID19C are presented in FIG. 17A. Results demonstrate that the prime-boost vaccination led to a significant increase in the effector memory cells, in both short-lived effector cell population and effector memory cell population. A significant increase in central memory T cells following the administration of a booster dose was noted in young mice, however this difference was not observed in aging mice.

### Results: Homologous Prime-Boost Increases T Cell Memory

In both the young and aging mice, a single shot of the SCV-COVID19C vaccine induces a significant increase in spike-specific IFN-γ T cell response compared to the vector only control mice. The administration of a booster dose resulted in significant increase in the spike-specific IFN-γ T cell and this was observed in both the young and aging mice (FIG. 17B).

Further profiling of the spike specific T cell responses as S1-, RBD- or S2-specific was done by re-stimulating the cells with peptide pools spanning the S1 region (sans the RBD; Pool 1), RBD and remaining S1 region (Pool 2) and S2 region (Pool 3). IFN-γ T cell responses directed towards S1, RBD and S2 regions were detected both in young and aging mice, indicating a wide breadth of T cell responses. IFN-γ producing T cell responses were predominantly directed towards the RBD region, as evidenced by IFN-γ spot-forming units by ELISpot (FIG. 17C), percentage of IFN-γ producing CD8 T cells (FIG. 17D) and number of triple cytokine positive (IFN-γ, TNF-α, IL2) producing CD8 T (FIG. 17E) by intracellular cytokine staining. As expected, a significant boost in the antigen-specific T cell population was observed in the prime-boost regimen compared to the single-dose vaccination.

### Conclusion

Vaccination with SCV-COVID19C induces a polyfunctional CD8 T cell responses directed towards both subunits of the spike protein (S1 and S2; predominantly directed towards the RBD region of the S1 subunit) in both young and aging mice following a single shot vaccination regimen. Significant increase in the antigen-specific IFN-γ-producing T cells responses and effector memory populations was observed in both young and aging mice following a homologous prime-boost vaccination strategy.

### Example 11

Homologous Prime-Boost of SCV-COVID19C Potentially can Potentially Cross-React with SARS-CoV Based on CD8 T Cell Epitopes in the Spike RBD

### Experimental Strategy

6-9 week old female mice and 9-10 month old mice were vaccinated with SCV-COVID19C or vector only control by intramuscular administration at the dose of 10⁷ PFU/mouse and epitope-specific T cell responses were analyzed by ELISpot at day 7 post-vaccination.

### Method

ELISpot assay (described previously) and intracellular cytokine staining (described previously) were used to quantify IFN-γ-producing T cells following restimulation with two CD8 T cell epitopes in the RBD region that are 100% conserved between SARS-CoV and SARS-CoV-2 (VVLSFELL (SEQ ID NO:10) and VNFNFNGL (SEQ ID NO:11)).

### Result

A significant increase in the epitope-specific IFN-γ producing T cell responses was detected in SCV-COVID19C vaccinated mice compared to the vector control both by ELISpot (FIG. 18A) and ICS (FIG. 18B).

### Conclusion

The generation of IFN-γ producing T cell responses directed towards CD8 T cell epitopes VVLSFELL (SEQ ID NO: 10) and VNFNFNGL (SEQ ID NO:11) (that are conserved between SARS-CoV and SARS-CoV-2) following vaccination with SCV-COVID19C suggest that the vaccine may be capable of providing prophylactic protection against both SARS-CoV and SARS-CoV-2.

### Example 12

Single Vaccination with SCV-COVID19A Generates Spike- and Membrane-Specific CD8 T Cell Responses

### Experimental Strategy

Groups of 6-9 week old male C57BL/6 mice (n=5 mice per group) were vaccinated via intramuscular administration with SCV-COVID19A (10⁷ PFU/mouse) or the vector control SCV-SMX06. Blood samples were collected 21 days post-vaccination and T cell responses directed towards the spike, membrane, and nucleocapsid proteins were detected by ELISpot.

### Method

ELISpot assay (described previously) was used to quantify IFN-γ-producing T cells. To evaluate the nature of the IFN-γ T cell responses elicited by SCV-COVID19A vaccination, we restimulated the CD8 T cells with three different peptide pools spanning the sequence of the following regions: the S1 sans the RBD, RBD and remaining S1 region after RBD, and the S2. To determine whether CD8 T cell responses targeting the membrane or nucleocapsid were generated, cells were re-stimulated with peptide pools spanning the sequences of the membrane protein and nucleocapsid proteins.

### Result

A significant increase in spike-specific IFNγ⁺ producing T cell responses was detected in SCV-COVID19A vaccinated mice compared to the vector control across the S1, RBD and S2 regions (FIG. 19A). Membrane-specific IFNγ⁺ producing T cell response was shown to be significantly higher compared to the vector control (FIG. 19B), while nucleocapsid-specific T cell response was comparable between the SCV-COVID19A vaccinated mice and the vector control (FIG. 19C).

### Conclusion

A single dose of SCV-COVID19 vaccination induces a wide breadth of T cell responses as shown by the increase in spike-specific and membrane-specific IFNγ-secreting CD8+ T cells.

### Example 13

Single Vaccination with Equal Proportions of SCV-COVID19C and SCV-COVID19G Induces Spike-Specific Antibody Responses and CD8+ T Cell Responses Directed Towards the Spike and Membrane Proteins

### Experimental Strategy

Groups of 6-9 week old male C57BL/6 mice (n=5 mice per group) were vaccinated via intramuscular administration with a mixed vaccine containing equal proportions of SCV-COVID19C (10⁷ PFU/mouse) and SCV-COVID19G (10⁷ PFU/mouse) or the vector control SCV-SMX06. Blood samples were collected 21 days post-vaccination and serum isolated for antibody analysis. Spike (S1)-specific antibody responses were determined by ELISA and T cell responses directed towards the spike and membrane protein were detected by ELISpot and ICS.

### Method

Enzyme-linked immunosorbent assay for S1-specific antibody levels was performed as described previously. ELISpot assay (described previously) was used to identify spike and membrane specific IFN-γ producing T cell responses using peptides spanning the entire length of the protein.

### Results

S1-specific antibody responses were evaluated by ELISA at 21 days post-immunization. Vaccination with the mixed vaccine comprised of SCV-COVID19C and SCV-COVID19G generated significantly higher S1-specific antibody response compared to the vector only SMX06 control (FIG. 20A). Level of spike-specific IFNγ⁺ producing T cell detected by ICS following a single shot vaccination the mixed vaccine was also higher compared to the vector control (FIG. 20B). Vaccination with the mixed vaccine led to a significant increase in antigen-specific spot-forming units (SFU) against peptides spanning the full length of spike and membrane protein compared to the vector control (FIG. 20C).

### Conclusion

Example 13 herein indicate that immunization of animal model with a mixed vaccine comprised of SCV-COVID19C and SCV-COVID19G encoding SARS-CoV-2 spike protein and SARS-CoV-2 membrane and nucleocapsid proteins, respectively, induces cellular and humoral responses as shown by the production of S1-specific antibodies, and increase in spike-specific IFNγ-secreting CD8+ T cells. These suggest that a mixed SCV-COVID19 vaccine may provide prophylactic protection against SARS-CoV-2, the infectious agent for COVID-19.

### Example 14

To Determine if Expressing Multiple Dominant Antigens from the Same Vector is not Detrimental in Stimulating Optimal Immune Responses as Compared to Expressing Each Dominant Antigen from a Single Vector.

When combining different live attenuated viral vaccines, competition between the viruses is the most frequently observed problem. It can be circumvented by increasing the dose of the combination vaccine or adjusting the dosage of each vaccine component to overcome competition from the dominant vaccine(s) components.

Antigenic competition by ‘immunological interference’ has been reported between components of the trivalent diphtheria-pertussis-tetanus vaccine, between canine distemper bacterins and live canine distemper virus and when Bordetella is used as a diluent for live combination distemper virus, adenovirus type 2, parvovirus, and parainfluenza virus vaccines. In some cases, inoculation with the multicomponent vaccine elicits less antibody than when the components are administered alone. In other cases the response to one antigen dominates while the responses to the others are suppressed. In other cases, where mutual competition occurs, the response to all components is reduced.

The degree of antigenic competition has been shown to be dependent on a number of parameters of vaccination, including the relative sites of inoculation of the competing antigens, the time interval between administration of the antigens and the dose of the dominant antigen relative to the suppressed antigen.

Even though the above can be solved by expressing the immunizing antigens from several disease causing agent from the same vector, thereby ensuring equal representation of each immunizing antigen to the immune system and with a single vector for antigen delivery only the optimal route of vaccination for the vector only needs to be considered, there is the risk that antigen interference can occur by preferential antigen capture and MHC presentation by antigen presenting cells such as B cells and dendritic cells. An antigen with dominant B cell and or T cell epitopes will produce a stronger immune response than an antigen that have sub-optimal B-cell or T-cell epitopes. Therefore this would lead to a biased immune response to the most dominant antigen when vaccinated with a single vector that expresses multiple antigens from multiple disease causing agents.

A study is carried out to determine if the expression of multiple dominant antigens from multiple disease causing virus will interfere with each other's immune response. Expressing two dominant antigens from the same vector may interfere with each other's capacity to stimulate a potent immune response to their respective viruses, i.e. one dominant antigen may have more dominance over the other.

To determine if expressing multiple dominant antigens from the same vector is not detrimental in stimulating optimal immune responses as compared to expressing each dominant antigen from a single vector, a vaccination study in mice is carried out.

### Experimental Strategy

Wildtype C57BL/6 and interferon receptor deficient mice (IFNAR) female mice or ACE-2-deficient mice are vaccinated once with a recombinant virus SCV-COVID19A or SCV-COVID19B, or mixtures of SCV-COVID19C, SCV-COVID19D, or SCV-COVID19E, or empty vector control in groups of 6 mice per treatment group. All treatment groups are given 10⁶ PFU/mouse of vaccine via intraperitoneal injections and bled at 2 and 4 weeks post-vaccination. All mice are challenged at 6 weeks post-vaccination.

### Neutralization Assay

Levels of neutralizing antibodies are often used as a correlate of protection. Therefore levels of neutralizing antibodies was calculated prior to challenge in all vaccine groups using a standard microneutralization assay on Vero cells against SARS-CoV-2. In brief, sera is heat inactivated (56° C. for 30 min) serum from each mouse is serial diluted in duplicate in 96 well plates and is incubated with 100 CCID1₅₀ units of virus for 1 hr at 37° C. Following this neutralization step, freshly split Vero cells are overlaid (10⁴ cells per well) onto the serum/virus mixture and incubated for 5 days until cytopathic effects are visualized under a microscope. The serum dilution fiving 100% protection against cytopathic effect is determined using crystal violet staining.

### Results

Both vaccine candidates expressing SARS-CoV-2 antigens induce neutralizing antibodies against SARS-CoV-2 virus after a single administration of vaccine.

Protection of Mice Foetuses from SARS-CoV-2 Virus Infection Mothers that had Previously been Vaccinated with Recombinant SCV-COVID19 Viruses Before Pregnancy.

The aim of this study is to show that previous vaccination of female mice expressing SARS-CoV-2 antigens prior to pregnancy can afford protection against SARS-CoV-2 virus infection of their unborn foetuses.

This study is carried out by vaccinating female IFNAR −/− with SCV-COVID19A, SCV-COVID19B, or vector only followed by mating with male IFNAR mice. Pregnant mice are then infected with SARS-CoV-2.

### Experimental Strategy

6-8 week IFNAR −/− are vaccinated once via the intramuscular route with either the single-vectored vaccine, SCV-COVID19A, SCV-COVID19B, or vector only at week 0 at 10⁶ PFU/mouse. Groups of mice are bled at 4 weeks post-vaccination to check for seroconversion to the vaccine. At 6 weeks post-vaccination, timed matings are initiated to induce pregnancy in vaccinated mice. Female mice are checked daily for evidence of successful pregnancy (vaginal plugs). At embryonic day 6.5, pregnant mice are infected with SARS-CoV-2 at 10⁴ CCID50 units via subcutaneous infection. Following infection, pregnant mice are bled daily between days 1 to 5 to check for viraemia. At embryonic day 17.5, pregnant mice are culled and materials harvested to assess for infectious SARS-CoV-2.

### Results

Pregnant female mice previously vaccinated with SCV-COVID19A or SCV-COVID19B prior to becoming pregnancy are able to prevent SARS-CoV-2 virus replication during challenge with SARS-CoV-2 as shown by no detection of viraemia post-challenge. The SCV vector only vaccinated mice are not able to prevent viral replication with SARS-CoV-2 virus.

Female mice that are previously vaccinated with a single shot of SCV-COVID19A or SCV-COVID19B vaccine prior to mating and pregnancy show no detectable levels of SARS-CoV-2 virus after challenge. Vaccination prevents challenge virus from infecting the placenta and by doing so blocks onward transmission of SARS-CoV-2 virus to the vulnerable foetuses.

However, this is not the case for female mice previously vaccinated with SCV vector only where after challenge during pregnancy some of the placentas may become infected and transmission of SARS-CoV-2 virus infections to the foetuses may occur.

### Conclusion

Pregnant female mice previously vaccinated with a single shot of SCV-COVID19A or SCV-COVID19B single vectored vaccine are protected from SARS-CoV-2 challenge compared to the control vaccine which is shown by viraemia results.

Vaccination of the mother prior to pregnancy may afford protection to their unborn foetuses by preventing the SARS-CoV-2 virus from infecting the maternal placenta and blocking onwards transmission to fetal brain.

The onwards transmission of the SARS-CoV-2 challenge virus to the foetus is blocked by prior vaccination of mother before pregnancy.

### Example 15

Single Vaccination with SCV-COVD19C Generates Epitope-Specific Cytotoxic T Lymphocyte (CTL) Activity

CD8 cytotoxic T lymphocytes (CTLs) recognize class I MHC-associated peptides and, upon antigen-dependent stimulation, kill virus-infected cells by secreting granzymes and perforins. Virus-specific CTL responses play a critical role in containing viremia. Perforin creates cell membrane pores, allowing intracellular delivery of granzymes, leading to cleavage and activation of caspases that induce apoptotic death. In this example, we demonstrate the activation of effector T cells using radioactive isotope 51 Chromium release to assess virus-specific T cell-mediated cytotoxicity.

### Experimental Strategy

C57BL/6 mice were vaccinated via intramuscular administration with SCV-COVID19C or the vector control SCV-SMX06 at a dose of 10⁷ pfu per mouse. At day 7 post-vaccination, spleens were harvested and effector cells were assayed for direct ex vivo cytolytic T lymphocyte activity against peptide determinant-pulsed EL4 target cells via standard 51 Chromium (⁵¹Cr)-release.

### Cytolytic T Lymphocyte Assay

EL4 (H-2^{b}) cells were grown and prepared for assay via peptide pulsing and ⁵¹Cr labelling. EL4 cells were washed and pulsed for 2 hours with peptides representing SARS-CoV-2 immunodominant T cell epitopes, YNYLYRLF (SEQ ID NO:9) or VNFNFNGL (SEQ ID NO:11). These two epitopes are located in the RBD region of the 51 subunit of the SARS-CoV-2 spike protein. The cells were mixed every 20 minutes by gentle tapping. Peptide pulsed EL4 cells were then washed twice to remove any excess peptide and labelled with 20-50 μCi of ⁵¹Cr for 45-60 minutes. Cells were washed twice to remove excess ⁵¹Cr and resuspended at 2×10⁴ peptide pulsed, radio-labelled target cells/100 μl volume.

Effector cells were prepared from harvested spleens of vaccinated animals. Dilutions of single cell suspension of splenocytes were prepared by passing cells through 70 μM cell strainers and ACK lysis prior to resuspension in complete media. Cells were resuspended at 2×10⁷ cells/ml and dispensed into wells in a 3-fold serial dilution.

Target cells were added into wells containing the effector cells and incubated for 6 hours. For the maximum release control, 100 μl of Triton X was added to the wells to lyse the cells and completely release chromium into the medium. After incubation, the plate was spun at 1200 rpm for 5 minutes and 30 μI of the supernatants were transferred to a Luma plate for measurement of radioactivity. The plate was allowed to dry overnight and evaluated on the Microbeta2 plate reader the next day. Percent lysis for each concentration of effector cells is determined using the following formula:

Percent Specific Lysis=[Sample ⁵¹Cr release(cpm)−Spontaneous release(cpm)]/[Maximum release(cpm)−Spontaneous release(cpm)]×100%.

### Results

Results show that that there is specific lysis of target cells pulsed with the peptides YNYLYRLF (SEQ ID NO:9) and VNFNFNGL (SEQ ID NO:11) (FIG. 21A,B) suggesting that the vaccine generates epitope-specific cytotoxic T cells. The results look epitope-specific with no lysis detected for the control mice and control target cells (SMX06 vaccinated mice and non-pulsed target cells, respectively) (FIG. 21C).

### Conclusion

A single vaccination of SCV-COVID19C generates SARS-CoV-2 epitope-specific cytolytic T lymphocyte response.

### BIBLIOGRAPHY

Altschul et al. (1997) Nucleic Acids Res. 25:3389-3402
Ausubel et al. (1999) Current Protocols in Molecular Biology (Supplement 47), John Wiley & Sons, New York
Boshart et al. (1985) Cell 41:521
Brooks et al. (1995) J. Virol. 69(12):7688-7698
Caddy et al. (2020) EMBO J. 40: e106228
Chen et al. (2020) Lancet 395(10223):507-513
Dijkema et al. (1985) EMBO J. 4:761
Drillien R et al. (1978) J. Virol. 28(3):843-850
Eldi et al. (2017) Mol. Ther. 25:2332-2344
Gorman et al. (1982) Proc. Natl. Acad. Sci. USA 79:6777
Ham (1965) Proc. Natl. Acad. Sci. USA 53:288-293
Hammond et al. (1997) J. Virol. Methods 66(1):135-138
Hsiao et al. (2006) J. Virol. 80(15):7714-7728
Howley, Knipe (2020) Fields Virology Volume 1: Emerging Viruses, 7^{th} Ed., Wolters Kluwer Health, Philadelphia, Pennsylvania
Kibler et al. (2011) Plos One 6(11)
Lu et al. (2020) Lancet 395(10224):565-574
Meisinger-Henschel et al. (2007) J. Gen. Virol. 88(12):3249-3259
Murphy et al. (1995) Virus Taxonomy Springer Verlag: 79-87
Padron-Regalado (2020) Infect. Dis. Ther. 9:255-274
Prow et al. (2020) NPJ Vaccines 5:44
Prow et al. (2018) Nat. Commun. 9:1230
Puck et al. (1958) J. Exp. Med. 108:945-956
Sambrook et al. (1989) Molecular Cloning: A Laboratory Manual, 2^{nd} Ed., Cold Spring Harbor Press, Plainsview, New York
Shisler et al. (2004) J. Virol. 78(7):3553-3560
Siu et al. (2008) J. Virol. 82(22):11318-11330
Spehner et al. (1998) J. Virol. 62(4):1297-1304
Sohrabi et al. (2020) Int J Surg 76:71-76
Werden et al. (2008) Chapter 3: Poxvirus Host Range Genes. In: Advances in Virus Research, 71
Yuki et al. (2020) Clin. Immunol. 215:108427
Zhang et al. (2020) Emerg, Microbes Infect. 9(1):386-389
Zhou et al. (2020) Nature 579(7798):270-273

1. A composition for raising an immune response in an animal which prevents or decreases the risk of SARS-CoV-2 coronavirus disease, the composition comprising a genetically engineered attenuated vaccinia virus, wherein the vaccinia virus genome comprises a nucleic acid sequence encoding at least one human coronavirus SARS-CoV-2 polypeptide selected from the group consisting of a spike protein polypeptide or an immunogenic part thereof, a membrane protein polypeptide or an immunogenic part thereof, a nucleocapsid protein polypeptide or an immunogenic part thereof, and an envelope protein polypeptide or an immunogenic part thereof, wherein the attenuated vaccinia virus comprises a deletion of at least one gene which encodes an endogenous essential assembly or maturation protein.

2. The composition of claim 1, wherein the attenuated vaccinia virus genome comprises a nucleic acid sequence encoding a human coronavirus SARS-CoV-2 spike protein polypeptide or an immunogenic part thereof.

3. The composition of claim 1, wherein the attenuated vaccinia virus genome comprises a nucleic acid sequence encoding a human coronavirus SARS-CoV-2 membrane protein polypeptide or immunogenic part thereof.

4. The composition of claim 1, wherein the attenuated vaccinia virus genome comprises a nucleic acid sequence encoding a human coronavirus SARS-CoV-2 nucleocapsid protein polypeptide or immunogenic part thereof.

5. The composition of claim 1, wherein the attenuated vaccinia virus genome comprises a nucleic acid sequence encoding a human coronavirus SARS-CoV-2 membrane protein polypeptide or immunogenic part thereof and nucleocapsid protein polypeptide or immunogenic part thereof.

6. The composition of claim 1, wherein the attenuated vaccinia virus genome comprises a nucleic acid sequence encoding a spike protein polypeptide or an immunogenic part thereof, and membrane protein polypeptide or an immunogenic part thereof and nucleocapsid protein polypeptide or immunogenic part thereof, of human coronavirus SARS-CoV-2.

7. The composition of claim 1, wherein the attenuated vaccinia virus genome comprises a nucleic acid sequence encoding a human coronavirus SARS-CoV-2 spike polypeptide or immunogenic part thereof, and a membrane protein polypeptide or immunogenic part thereof, and a nucleocapsid protein polypeptide or immunogenic part thereof, and an envelope protein polypeptide or immunogenic part thereof.

8. The composition of claim 1, wherein the nucleic acid sequence encoding at least one human coronavirus SARS-CoV-2 polypeptide is inserted into deleted ORFs of one or more immune modulatory genes selected from the group consisting of COP-C23L, COP-B29R, COP-C3L, COP-N1 L, COP-A35R, COP-A39R, COP-A41L, COP-A44R, COP-A46R, COP-B7R, COP-B8R, COP-B13R, COP-B16R, and COP-B19R.

9. The composition of claim 1, wherein the nucleic acid sequence encoding at least one human coronavirus SARS-CoV-2 polypeptide is inserted into an intergenic region (IGR) of the attenuated vaccinia virus genome, wherein the IGR is located between or is flanked by two adjacent ORFs of the vaccinia virus genome.

10. The composition of claim 9, wherein the IGR of the attenuated vaccinia virus genome is selected from the group consisting of F9L-F10L, F12L-F13L, F17R-E1L, E1L-E2L, E8R-E9L, E9L-E10R, I1L-12L, 12L-13L, 15L-16L, 16L-17L, 17L-I8R, I8R-G1L, G1L-G3L, G3L-G2R, G2R-G4L, G4L-G5R, G5R-G5.5R, G5.5R-G6R, G6R-G7L, G7L-G8R, G8R-G9R, G9R-L1R, L1R-L2R, L2R-L3L, L3L-L4R, L4R-L5R, L5R-J1R, J3R-J4R, J4R-J5L, J5L-J6R, J6R-H1L, H1L-H2R, H2R-H3L, H3L-H4L, H4L-H5R, H5R-H6R, H6R-H7R, H7R-D1R, D1R-D2L, D2L-D3R, D3R-D4R, D4R-D5R, D5R-D6R, D6R-D7R, D9R-D10R, D10R-D11L, D11L-D12L, D12L-D13L, D13L-A1L, A1L-A2L, A2L-A2.5L, A2.5L-A3L, A3L-A4L, A4L-A5R, A5R-A6L, A6L-A7L, A7L-A8R, A8R-A9L, A9L-A10L, A10L-A11R, A11R-A12L, A12L-A13L, A13L-A14L, A14L-A14.5L, A14.5L-A15L, A15L-A16L, A16L-A17L, A17L-A18R, A18R-A19L, A19L-A21L, A21L-A20R, A20R-A22R, A22R-A23R, A23R-A24R, A28L-A29L and A29L-A30L, 001L-002L, 002L-003L, 005R-006R, 006L-007R, 007R-008L, 008L-009L, 017L-018L, 018L-019L, 019L-020L, 020L-021L, 023L-024L, 024L-025L, 025L-026L, 028R-029L, 030L-031L, 031 L-032L, 032L-033L, 035L-036L, 036L-037L, 037L-038L, 039L-040L, 043L-044L, 044L-045L, 046L-047R, 049L-050L, 050L-051 L, 051 L-052R, 052R-053R, 053R-054R, 054R-055R, 055R-056L, 061L-062L, 064L-065L, 065L-066L, 066L-067L, 077L-078R, 078R-079R, 080R-081R, 081R-082L, 082L-083R, 085R-086R, 086R-087R, 088R-089L, 089L-090R, 092R-093L, 094L-095R, 096R-097R, 097R-098R, 101R-102R, 103R-104R, 105L-106R, 107R-108L, 108L-109L, 109L-110L, 110L-111L, 113L-114L, 114L-115L, 115L-116R, 117L-118L, 118L-119R, 122R-123L, 123L-124L, 124L-125L, 125L-126L, 133R-134R, 134R-135R, 136L-137L, 137L-138L, 141L-142R, 143L-144R, 144R-145R, 145R-146R, 146R-147R, 147R-148R, 148R-149L, 152R-153L, 153L-154R, 154R-155R, 156R-157L, 157L-158R, 159R-160L, 160L-161R, 162R-163R, 163R-164R, 164R-165R, 165R-166R, 166R-167R, 167R-168R, 170R-171R, 173R-174R, 175R-176R, 176R-177R, 178R-179R, 179R-180R, 180R-181R, 183R-184R, 184R-185L, 185L-186R, 186R-187R, 187R-188R, 188R-189R, 189R-190R and 192R-193R.

11. The composition of claim 1, wherein the attenuated vaccinia virus comprises deletion of one or more genes selected from the group consisting of a vaccinia virus A41L gene, a vaccinia virus D13L gene, vaccinia virus B7R-B8R genes, a vaccinia virus A39R gene and a vaccinia virus C3L gene.

12. The composition of claim 11, wherein the at least one nucleic acid sequence encoding a human coronavirus SARS-CoV-2 polypeptide is inserted into at least one deletion site of the one or more genes.

13. The composition of claim 12, wherein a human coronavirus SARS-CoV-2 spike protein polypeptide, or an immunogenic part thereof, is inserted into a vaccinia virus A41 L gene deletion site.

14. The composition of claim 12, wherein a human coronavirus SARS-CoV-2 membrane protein polypeptide or an immunogenic part thereof, and a nucleocapsid protein polypeptide or an immunogenic part thereof, is inserted into a vaccinia virus D13L gene deletion site.

15. The composition of claim 12, wherein a human coronavirus SARS-CoV-2 envelope protein polypeptide, or an immunogenic part thereof, is inserted into vaccinia virus B7R-B8R gene deletion site.

16. The composition of claim 12, wherein the human coronavirus SARS-CoV-2 polypeptide is encoded by one or more expression cassettes having a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7 and SEQ ID NO:8.

17. The composition of claim 1, comprising a pharmaceutically acceptable carrier or diluent.

18. A composition for raising an immune response in animal which decreases the risk of a coronavirus disease, the composition comprising a genetically engineered attenuated vaccinia virus, wherein the vaccinia virus genome comprises a nucleic acid sequence encoding a spike protein polypeptide or an immunogenic part thereof, of human coronavirus SARS-CoV-2, and wherein the attenuated vaccinia virus comprises a deletion of at least one gene which encodes an endogenous essential assembly or maturation protein, admixed with a second genetically engineered attenuated vaccinia virus, wherein the second vaccinia virus genome comprises a nucleic acid sequence encoding a membrane protein polypeptide and nucleocapsid protein polypeptide or immunogenic part or parts thereof, of human coronavirus SARS-CoV-2, and wherein the second attenuated vaccinia virus comprises a deletion of at least one gene which encodes an endogenous essential assembly or maturation protein.

19. A genetically engineered attenuated vaccinia virus vector, wherein the vaccinia virus genome comprises a nucleic acid sequence encoding a spike protein polypeptide, a membrane protein polypeptide and a nucleocapsid protein polypeptide, and/or an envelope protein polypeptide of human coronavirus SARS-CoV-2, wherein the attenuated vaccinia virus vector expresses the aforementioned polypeptides which assemble into virus-like-particles.

20. A method for preventing or decreasing the risk of SARS-CoV-2 infection comprising administering the composition of claim 1 to an animal, including a human, in an amount effective to elicit an immune response directed against SARS-CoV-2.
